SEQUENCE LISTING (13 sequences)

SEQ ID NO: 1 - RNA, 31 nt, synthetic construct
  source 1..31: mol_type = other RNA
aggagaagctgtctatcgggctccagcggtc

SEQ ID NO: 2 - RNA, 56 nt, synthetic construct
  source 1..56: mol_type = other RNA
gccgccgctccagctttgcacgtttcgatcccaaaggccctttttagggccgacca

SEQ ID NO: 3 - RNA, 207 nt, synthetic construct
  source 1..207: mol_type = other RNA
agaacgcccccaaaatctgtttctaattttacagaaatcttttgaaacttggcacggtattcaaaagtccgtggaaagaaaaaaaccttgtcctggcttcagcttccaactacaaagacagacttggtccttttcaacggttttcacagatccagtgacccacgctctgaagacagaattagctaactttcaaaaacatctggaaaa

SEQ ID NO: 4 - RNA, 284 nt, synthetic construct
  source 1..284: mol_type = other RNA
aaacgatgcagatgcaagcctgtatcagaatctgaaaaccttatataaacccctcagacagtttgcttattttattttttatgttgtaaaatgctagtataaacaaacaaattaatgttgttctaaaggctgttaaaaaaaagatgaggactcagaagttcaagctaaatattgtttacattttctggtactctgtgaaagaagagaaaagggagtcatgcattttgctttggacacagtgttttatcacctgttcatttgaagaaaaataataaagtcagaag

SEQ ID NO: 5 - RNA, 54 nt, synthetic construct
  source 1..54: mol_type = other RNA
gttccctatcactctctttaatcactactcacagtaacctcaactcctgccaca

SEQ ID NO: 6 - RNA, 197 nt, synthetic construct
  source 1..197: mol_type = other RNA
ttaagtgcttcccacttaaaacatatcaggccttctatttatttaaatatttaaattttatatttattgttgaatgtatggtttgctacctattgtaactattattcttaatcttaaaactataaatatggatcttttatgattctttttgtaagccctaggggctctaaaatggtttcacttatttatcccaaaat

SEQ ID NO: 7 - RNA, 47 nt, synthetic construct
  source 1..47: mol_type = other RNA
aggaaataagagagaaaagaagagtaagaagaaatataagagccacc

SEQ ID NO: 8 - RNA, 110 nt, synthetic construct
  source 1..110: mol_type = other RNA
gctggagcctcggtggccaagcttcttgccccttgggcctccccccagcccctcctccccttcctgcacccgtacccccgtggtctttgaataaagtctgagtgggcggc

SEQ ID NO: 9 - RNA, 54 nt, synthetic construct
  source 1..54: mol_type = other RNA
gagaataaactagtattcttctggtccccacagactcagagagaacccgccacc

SEQ ID NO: 10 - RNA, 295 nt, synthetic construct
  source 1..295: mol_type = other RNA
ctcgagctggtactgcatgcacgcaatgctagctgcccctttcccgtcctgggtaccccgagtctcccccgacctcgggtcccaggtatgctcccacctccacctgccccactcaccacctctgctagttccagacacctcccaagcacgcagcaatgcagctcaaaacgcttagcctagccacacccccacgggaaacagcagtgattaacctttagcaataaacgaaagtttaactaagctatactaaccccagggttggtcaatttcgtgccagccacaccctggagctagc

SEQ ID NO: 11 - RNA, 10025 nt, synthetic construct
  source 1..10025: mol_type = other RNA
ataggcggcgcatgagagaagcccagaccaattacctacccaaaatggagaaagttcacgttgacatcgaggaagacagcccattcctcagagctttgcagcggagcttcccgcagtttgaggtagaagccaagcaggtcactgataatgaccatgctaatgccagagcgttttcgcatctggcttcaaaactgatcgaaacggaggtggacccatccgacacgatccttgacattggaagtgcgcccgcccgcagaatgtattctaagcacaagtatcattgtatctgtccgatgagatgtgcggaagatccggacagattgtataagtatgcaactaagctgaagaaaaactgtaaggaaataactgataaggaattggacaagaaaatgaaggagctcgccgccgtcatgagcgaccctgacctggaaactgagactatgtgcctccacgacgacgagtcgtgtcgctacgaagggcaagtcgctgtttaccaggatgtatacgcggttgacggaccgacaagtctctatcaccaagccaataagggagttagagtcgcctactggataggctttgacaccaccccttttatgtttaagaacttggctggagcatatccatcatactctaccaactgggccgacgaaaccgtgttaacggctcgtaacataggcctatgcagctctgacgttatggagcggtcacgtagagggatgtccattcttagaaagaagtatttgaaaccatccaacaatgttctattctctgttggctcgaccatctaccacgagaagagggacttactgaggagctggcacctgccgtctgtatttcacttacgtggcaagcaaaattacacatgtcggtgtgagactatagttagttgcgacgggtacgtcgttaaaagaatagctatcagtccaggcctgtatgggaagccttcaggctatgctgctacgatgcaccgcgagggattcttgtgctgcaaagtgacagacacattgaacggggagagggtctcttttcccgtgtgcacgtatgtgccagctacattgtgtgaccaaatgactggcatactggcaacagatgtcagtgcggacgacgcgcaaaaactgctggttgggctcaaccagcgtatagtcgtcaacggtcgcacccagagaaacaccaataccatgaaaaattaccttttgcccgtagtggcccaggcatttgctaggtgggcaaaggaatataaggaagatcaagaagatgaaaggccactaggactacgagatagacagttagtcatggggtgttgttgggcttttagaaggcacaagataacatctatttataagcgcccggatacccaaaccatcatcaaagtgaacagcgatttccactcattcgtgctgcccaggataggcagtaacacattggagatcgggctgagaacaagaatcaggaaaatgttagaggagcacaaggagccgtcacctctcattaccgccgaggacgtacaagaagctaagtgcgcagccgatgaggctaaggaggtgcgtgaagccgaggagttgcgcgcagctctaccacctttggcagctgatgttgaggagcccactctggaagccgatgtcgacttgatgttacaagaggctggggccggctcagtggagacacctcgtggcttgataaaggttaccagctacgatggcgaggacaagatcggctcttacgctgtgctttctccgcaggctgtactcaagagtgaaaaattatcttgcatccaccctctcgctgaacaagtcatagtgataacacactctggccgaaaagggcgttatgccgtggaaccataccatggtaaagtagtggtgccagagggacatgcaatacccgtccaggactttcaagctctgagtgaaagtgccaccattgtgtacaacgaacgtgagttcgtaaacaggtacctgcaccatattgccacacatggaggagcgctgaacactgatgaagaatattacaaaactgtcaagcccagcgagcacgacggcgaatacctgtacgacatcgacaggaaacagtgcgtcaagaaagaactagtcactgggctagggctcacaggcgagctggtggatcctcccttccatgaattcgcctacgagagtctgagaacacgaccagccgctccttaccaagtaccaaccataggggtgtatggcgtgccaggatcaggcaagtctggcatcattaaaagcgcagtcaccaaaaaagatctagtggtgagcgccaagaaagaaaactgtgcagaaattataagggacgtcaagaaaatgaaagggctggacgtcaatgccagaactgtggactcagtgctcttgaatggatgcaaacaccccgtagagaccctgtatattgacgaagcttttgcttgtcatgcaggtactctcagagcgctcatagccattataagacctaaaaaggcagtgctctgcggggatcccaaacagtgcggtttttttaacatgatgtgcctgaaagtgcattttaaccacgagatttgcacacaagtcttccacaaaagcatctctcgccgttgcactaaatctgtgacttcggtcgtctcaaccttgttttacgacaaaaaaatgagaacgacgaatccgaaagagactaagattgtgattgacactaccggcagtaccaaacctaagcaggacgatctcattctcacttgtttcagagggtgggtgaagcagttgcaaatagattacaaaggcaacgaaataatgacggcagctgcctctcaagggctgacccgtaaaggtgtgtatgccgttcggtacaaggtgaatgaaaatcctctgtacgcacccacctcagaacatgtgaacgtcctactgacccgcacggaggaccgcatcgtgtggaaaacactagccggcgacccatggataaaaacactgactgccaagtaccctgggaatttcactgccacgatagaggagtggcaagcagagcatgatgccatcatgaggcacatcttggagagaccggaccctaccgacgtcttccagaataaggcaaacgtgtgttgggccaaggctttagtgccggtgctgaagaccgctggcatagacatgaccactgaacaatggaacactgtggattattttgaaacggacaaagctcactcagcagagatagtattgaaccaactatgcgtgaggttctttggacttgatctggactccggtctattttctgcacccactgttccgttatccattaggaataatcactgggataactccccgtcgcctaacatgtacgggctgaataaagaagtggtccgtcagctctctcgcaggtacccacaactgcctcgggcagttgccactggaagagtctatgacatgaacactggtacactgcgcaattatgatccgcgcataaacctagtacctgtaaacagaagactgcctcatgctttagtcctccaccataatgaacacccacagagtgacttttcttcattcgtcagcaaattgaagggcagaactgtcctggtggtcggggaaaagttgtccgtcccaggcaaaatggttgactggttgtcagaccggcctgaggctaccttcagagctcggctggatttaggcatcccaggtgatgtgcccaaatatgacataatatttgttaatgtgaggaccccatataaataccatcactatcagcagtgtgaagaccatgccattaagcttagcatgttgaccaagaaagcttgtctgcatctgaatcccggcggaacctgtgtcagcataggttatggttacgctgacagggccagcgaaagcatcattggtgctatagcgcggcagttcaagttttcccgggtatgcaaaccgaaatcctcacttgaagagacggaagttctgtttgtattcattgggtacgatcgcaaggcccgtacgcacaatccttacaagctttcatcaaccttgaccaacatttatacaggttccagactccacgaagccggatgtgcaccctcatatcatgtggtgcgaggggatattgccacggccaccgaaggagtgattataaatgctgctaacagcaaaggacaacctggcggaggggtgtgcggagcgctgtataagaaattcccggaaagcttcgatttacagccgatcgaagtaggaaaagcgcgactggtcaaaggtgcagctaaacatatcattcatgccgtaggaccaaacttcaacaaagtttcggaggttgaaggtgacaaacagttggcagaggcttatgagtccatcgctaagattgtcaacgataacaattacaagtcagtagcgattccactgttgtccaccggcatcttttccgggaacaaagatcgactaacccaatcattgaaccatttgctgacagctttagacaccactgatgcagatgtagccatatactgcagggacaagaaatgggaaatgactctcaaggaagcagtggctaggagagaagcagtggaggagatatgcatatccgacgactcttcagtgacagaacctgatgcagagctggtgagggtgcatccgaagagttctttggctggaaggaagggctacagcacaagcgatggcaaaactttctcatatttggaagggaccaagtttcaccaggcggccaaggatatagcagaaattaatgccatgtggcccgttgcaacggaggccaatgagcaggtatgcatgtatatcctcggagaaagcatgagcagtattaggtcgaaatgccccgtcgaagagtcggaagcctccacaccacctagcacgctgccttgcttgtgcatccatgccatgactccagaaagagtacagcgcctaaaagcctcacgtccagaacaaattactgtgtgctcatcctttccattgccgaagtatagaatcactggtgtgcagaagatccaatgctcccagcctatattgttctcaccgaaagtgcctgcgtatattcatccaaggaagtatctcgtggaaacaccaccggtagacgagactccggagccatcggcagagaaccaatccacagaggggacacctgaacaaccaccacttataaccgaggatgagaccaggactagaacgcctgagccgatcatcatcgaagaggaagaagaggatagcataagtttgctgtcagatggcccgacccaccaggtgctgcaagtcgaggcagacattcacgggccgccctctgtatctagctcatcctggtccattcctcatgcatccgactttgatgtggacagtttatccatacttgacaccctggagggagctagcgtgaccagcggggcaacgtcagccgagactaactcttacttcgcaaagagtatggagtttctggcgcgaccggtgcctgcgcctcgaacagtattcaggaaccctccacatcccgctccgcgcacaagaacaccgtcacttgcacccagcagggcctgctcgagaaccagcctagtttccaccccgccaggcgtgaatagggtgatcactagagaggagctcgaggcgcttaccccgtcacgcactcctagcaggtcggtctcgagaaccagcctggtctccaacccgccaggcgtaaatagggtgattacaagagaggagtttgaggcgttcgtagcacaacaacaatgacggtttgatgcgggtgcatacatcttttcctccgacaccggtcaagggcatttacaacaaaaatcagtaaggcaaacggtgctatccgaagtggtgttggagaggaccgaattggagatttcgtatgccccgcgcctcgaccaagaaaaagaagaattactacgcaagaaattacagttaaatcccacacctgctaacagaagcagataccagtccaggaaggtggagaacatgaaagccataacagctagacgtattctgcaaggcctagggcattatttgaaggcagaaggaaaagtggagtgctaccgaaccctgcatcctgttcctttgtattcatctagtgtgaaccgtgccttttcaagccccaaggtcgcagtggaagcctgtaacgccatgttgaaagagaactttccgactgtggcttcttactgtattattccagagtacgatgcctatttggacatggttgacggagcttcatgctgcttagacactgccagtttttgccctgcaaagctgcgcagctttccaaagaaacactcctatttggaacccacaatacgatcggcagtgccttcagcgatccagaacacgctccagaacgtcctggcagctgccacaaaaagaaattgcaatgtcacgcaaatgagagaattgcccgtattggattcggcggcctttaatgtggaatgcttcaagaaatatgcgtgtaataatgaatattgggaaacgtttaaagaaaaccccatcaggcttactgaagaaaacgtggtaaattacattaccaaattaaaaggaccaaaagctgctgctctttttgcgaagacacataatttgaatatgttgcaggacataccaatggacaggtttgtaatggacttaaagagagacgtgaaagtgactccaggaacaaaacatactgaagaacggcccaaggtacaggtgatccaggctgccgatccgctagcaacagcgtatctgtgcggaatccaccgagagctggttaggagattaaatgcggtcctgcttccgaacattcatacactgtttgatatgtcggctgaagactttgacgctattatagccgagcacttccagcctggggattgtgttctggaaactgacatcgcgtcgtttgataaaagtgaggacgacgccatggctctgaccgcgttaatgattctggaagacttaggtgtggacgcagagctgttgacgctgattgaggcggctttcggcgaaatttcatcaatacatttgcccactaaaactaaatttaaattcggagccatgatgaaatctggaatgttcctcacactgtttgtgaacacagtcattaacattgtaatcgcaagcagagtgttgagagaacggctaaccggatcaccatgtgcagcattcattggagatgacaatatcgtgaaaggagtcaaatcggacaaattaatggcagacaggtgcgccacctggttgaatatggaagtcaagattatagatgctgtggtgggcgagaaagcgccttatttctgtggagggtttattttgtgtgactccgtgaccggcacagcgtgccgtgtggcagaccccctaaaaaggctgtttaagcttggcaaacctctggcagcagacgatgaacatgatgatgacaggagaagggcattgcatgaagagtcaacacgctggaaccgagtgggtattctttcagagctgtgcaaggcagtagaatcaaggtatgaaaccgtaggaacttccatcatagttatggccatgactactctagctagcagtgttaaatcattcagctacctgagaggggcccctataactctctacggctaacctgaatggactacgacatagtctagtccgccaagatggaatcaagaatttggtgtctcgttgtctgtgttaatctgtgcatagtgtgtttgggcgccgccgtgtctagttcttcaacccgcggtacatccgccactcactctcatcactctagtcatacgactagtgccgctcacagccggagtggtagtgtgtcccagcgcgttacctccagtcagacagtttcacatggagtcaatgagactatctataatacgaccctgaaatacggggacgtggtcggtgtaaatacaaccaaatatccttatcgcgtatgttcaatggctcaaggtactgatctcatccgctttgagcgaaacatagtgtgcacatctatgaaaccaattaatgaagatctggatgaggggataatggtagtctacaaacggaatattgtagcacatacattcaaggtacgggtttatcaaaaggtcctgacgtttagacgatcatacgcttacattcacacaacgtacctcttgggatccaacacagagtatgtcgcccctccaatgtgggagattcatcatattaactcccattcccagtgttattctagctacagccgggtaatcgccggaacggtgtttgtggcgtaccatcgggattcatacgagaacaagacgatgcagctgatgccggacgactactcaaatactcacagcacacggtacgtcacagtcaaggaccaatggcacagccgcggatccacatggttgtaccgagagacatgcaatttgaactgcatggtaaccattacaaccgcaaggtctaagtacccgtatcatttttttgccacttccaccggcgacgtggtcgacatttccccattttacaatgggaccaaccgaaacgcgtcatactttggcgagaatgctgacaaattcttcatatttcccaactacacgatcgtctcagattttggtcgaccgaacagcgcactcgaaactcatagacttgtcgcattcctggaaagagcggactccgtgatcagttgggacattcaggacgagaaaaatgtcacctgccaactcactttttgggaagcgtcagagcgcacgatacgcagtgaagccgaggattcctaccattttagtagcgcgaagatgacagctacattcctcagcaagaagcaagaggtcaacatgtccgatagcgccctcgactgtgtcagggacgaggcgatcaataaactgcagcaaatatttaatacatcctataaccagacttacgagaagtatggtaatgtaagtgtgtttgaaactacaggagggctggtggtattttggcagggaatcaagcaaaaatctcttgtagagctcgagagacttgctaacaggtcctctcttaaccttactcataatagaacaaagagatccaccgatggaaacaacgcgacccatctgagcaacatggaatcagtccataacttggtgtatgcgcaactccaattcacatatgatacgttgaggggctatattaacagggcactggctcagatagcggaggcttggtgcgtagatcagcgacggactctggaagttttcaaggaacttagtaagattaatccatcagccattctctctgccatctacaacaaaccaatagcggcgaggtttatgggtgatgtgctggggctggcgagttgcgtaacgatcaatcagactagcgttaaagtccttcgggacatgaatgtgaaagagagcccaggacggtgttactctaggccagttgttatcttcaatttcgctaactcctcctatgttcagtatggacagcttggcgaagacaatgagatactgcttggtaaccatcggacagaagagtgtcaattgccgtcactgaagatctttatcgctgggaactctgcctatgagtacgttgactatcttttcaagcggatgatcgatctgtcatcaatctctacagtagactccatgattgcgcttgatattgacccgctcgaaaacacggattttagagttcttgagctttattcccagaaagaattgagatcctctaacgtctttgatcttgaggaaataatgagagaattcaattcttacaagcaacgagtcaagtatgtcgaggacaaggttgtagatccgcttcctccatacctgaagggtctcgatgatctgatgtccggtcttggggctgcgggtaaggcagtgggggttgccataggcgcggttgggggagcagtagcgagtgtagtcgagggggtggctactttccttaagaaccccttcggggcatttactatcatactcgtagcaatagccgtggtgatcataacatatttgatatacacgtgataaggcgcgcccacccagcggccgcatacagcagcaattggcaagctgcttacatagaactcgcggcgattggcatgccgccttaaaatttttattttatttttcttttcttttccgaatcggattttgtttttaatatttc

SEQ ID NO: 12 - RNA, 207 nt, synthetic construct; identical to SEQ ID NO: 3
  source 1..207: mol_type = other RNA

SEQ ID NO: 13 - RNA, 284 nt, synthetic construct; identical to SEQ ID NO: 4
  source 1..284: mol_type = other RNA

(19) United States
(12) Patent Application Publication
(10) Pub. No.: US 2025/0375390 A1
(43) Pub. Date: Dec. 11, 2025

(54) Method of Lyophilisation

(71) Applicant: GlaxoSmithKline Biologicals SA, Rixensart (BE)

(72) Inventors: Frédéric MATHOT, Rixensart (BE); Frederik LALEMAN, Rixensart (BE); Erwan BOURLES, Rixensart (BE)

(73) Assignee: GlaxoSmithKline Biologicals SA, Rixensart (BE)

(21) Appl. No.: 19/298,524

(22) Filed: Aug. 13, 2025

Related U.S. Application Data
(63) Continuation of application No. PCT/IB2024/051928, filed on Feb. 28, 2024.

(30) Foreign Application Priority Data
Mar. 1, 2023 (GB) 2303019.0

(51) Int. Cl. A61K 9/51; A61K 9/19; A61K 31/7105

(52) U.S. Cl. CPC A61K 9/5123; A61K 9/19; A61K 31/7105

(57) ABSTRACT

The present invention provides lyophilised pharmaceutical compositions and methods of making said lyophilised pharmaceutical compositions. More particularly, the present invention provides lyophilised pharmaceutical compositions comprising nucleic acid and lipid carrier particles and methods of making said lyophilised pharmaceutical compositions. The provided lyophilised compositions have improved critical quality attributes (CQAs) and the provided methods prevent the need for a deep-freeze cold chain. The present invention further provides the use of said lyophilised pharmaceutical compositions in medicine.

### CROSS-REFERENCE TO RELATED APPLICATIONS

This application is a continuation application under 35 U.S.C. § 365 (c) of International Application No. PCT/IB2024/051928, filed Feb. 28, 2024, which claims priority under 35 U.S.C. § 119 (b) to United Kingdom Patent Application No. 2303019.0, filed Mar. 1, 2023. The complete contents of each of the above-noted applications are hereby incorporated by reference for all purposes in their entireties.

### SEQUENCE LISTING INCORPORATION BY REFERENCE

This application contains a Sequence Listing, which has been submitted electronically in computer readable form in an XML format and which is hereby incorporated by reference in its entirety. Said XML file, created on Feb. 28, 2024, is named “70323WO.xml”, and is 23,841 bytes in size.

### TECHNICAL FIELD

The present invention relates to the field of lyophilised pharmaceutical compositions and methods of making and using said lyophilised pharmaceutical compositions. More particularly, the present invention relates to lyophilised pharmaceutical compositions comprising nucleic acid and lipid carrier particles and methods of making and using said lyophilised pharmaceutical compositions.

### BACKGROUND OF THE INVENTION

A major challenge of pharmaceutical compositions that comprise nucleic acids (e.g., mRNA vaccines, oligonucleotide therapeutics such as siRNA, antisense oligonucleotides etc.) is their instability, for example due to their susceptibility to hydrolysis. To avoid such degradation, said compositions are typically stored at minus 20° C. to minus 80° C. As a result, the compositions are subject to cold chain manufacture, distribution, and storage. The “cold-chain” acts to preserve biological product quality from the time of manufacture until the point of administration by ensuring that the pharmaceutical is stored and transported within the recommended temperature ranges.

However, maintaining cold chain storage has a number of disadvantages, including cost, complexity of distribution, and potential loss of product. In some cases, failure to maintain cold chain storage, e.g., due to refrigeration failure, electrical outages, etc., necessitate discarding of the pharmaceutical composition. In fact. Globally, about half of the vaccines are wasted due to improper temperature control (WHO, “Monitoring Vaccine Wastage at Country Level: Guidelines for Programme Managers”. World Health Organization; Geneva, Switzerland: 2005).

This issue was particularly highlighted for vaccines developed in response to the COVID-19 pandemic. Among the COVID-19 vaccines licensed for use in the United States, messenger RNA (mRNA)-based had the highest efficacy (Baden et al. N Engl J Med. 2021 Feb. 4; 384 (5): 403-416). In a pandemic scenario, mRNA vaccines may also be seen as preferred (e.g., over traditional vaccine) as they can be rapidly developed, with faster manufacturing times (Zhang et al. A thermostable mRNA vaccine against COVID-19. Cell. 2020; 182:1271 1283). Despite these positives, instability and ultracold storage requirements of mRNA vaccines remain major limitations and such limitations slow down the distribution of nucleic acid-based pharmaceuticals predominantly in resource poor countries of the world. Maintaining ultra-cold storage conditions is expensive and difficult to arrange in areas of the world with limited resources. Accordingly, methods to lyophilize (also referred to as freeze-dry) nucleic acid containing pharmaceutical products in order to reduce and/or prevent the need for the cold chain would be desirable.

Lyophilisation (freeze-drying) is commonly used in the pharmaceutical industry to increase the stability and shelf life of various products by removing the solvent (most usually water) from drug formulations (Chen et al. J Control Release. 2010 Mar. 19; 142 (3): 299-311). However, lyophilisation of nucleic acid containing pharmaceuticals, especially pharmaceutical compositions comprising nucleic acids that are encapsulated within lipid carrier particles (e.g., lipid nanoparticles) is not straightforward. Lyophilisation processes are time-consuming and can result in high inherent heterogeneity (i.e., vial-to-vial inconsistencies) due to vial location on shelves (edge vial effect) during the sublimation phase and the stochastic nucleation of ice during the freezing step.

Furthermore, it is crucial that certain critical quality attributes (CQAs) are retained during the freeze-drying of pharmaceutical compositions comprising nucleic acid that are encapsulated within lipid carrier particles. Some of the CQAs that should be maintained include, for example, one or more of percentage encapsulation, carrier particle size, polydispersity index (PDI), in vitro relative potency and purity. Negatively impacting one or more CQAs can lead to sub-optimal pharmaceutical compositions and loss of biological potency.

In particular, lyophilisation is known to impact the percentage encapsulation CQA. In other words, a certain amount of nucleic acid leaks from lipid carrier particles during the freeze-drying process thus leading to loss of payload encapsulation efficiency.

There remains a need for improved methods of lyophilizing pharmaceutical compositions (particularly vaccine compositions) containing nucleic acid encapsulating lipid carrier particles, such that the resulting lyophilised product retains its CQAs.

### SUMMARY OF THE INVENTION

The inventors of the present application have discovered improved methods for lyophilizing pharmaceutical compositions comprising nucleic acid encapsulated lipid carrier particles. In particular the inventors discovered that increasing the surface area to volume ratio of the pharmaceutical composition prior to, or during, the freeze-cycle, allows for much faster sublimation of ice crystals, much faster desorption and thus much faster drying cycles, compared to traditional batch freeze-drying processes. To the inventors' surprise, the resulting lyophilised product had significantly improved payload encapsulation efficiency compared to batch freeze drying.

Payload encapsulation efficiency of, for example, RNA vaccines is important. In order to reach the cytosol where ribosomes translate the RNA into the protein of interest, the RNA needs to remain intact and protected by its lipid carrier. Unentrapped RNA is susceptible to being rapidly broken down by nucleases within the extracellular medium following administration thus preventing the RNA from being further processed.

The inventors also found that the improved methods had certain advantages compared to batch freeze-drying processes for example, they were amenable to continuous/semi-continuous manufacturing processes, had better capacity-to-footprint ratio, were more environmentally friendly due to energy rationalization and resulted in improved production homogeneity where vials are monitored by process analytical technology (PAT) and processed individually (versus batch-mode process for standard lyophilisation with inherent heterogeneity due to vial location onto the shelves).

Accordingly, in a first aspect there is provided a pharmaceutical composition said pharmaceutical composition comprising a nucleic acid and lipid carrier particles, wherein the pharmaceutical composition is lyophilised and wherein either
(a) the percentage of nucleic acid that is encapsulated within the lipid carrier particles is greater than 75% of the total nucleic acid in the pharmaceutical composition; and/or
(b) the percentage of nucleic acid that remains encapsulated within the lipid carrier particles is reduced by less than 15% compared to the percentage of nucleic acid that is encapsulated within the lipid carrier particles in the pharmaceutical composition prior to lyophilisation.

In a second aspect there is provided a vaccine comprising the pharmaceutical composition of the first aspect.

In a third aspect there is provided a method of reconstituting the pharmaceutical composition of the first aspect, comprising adding a sterile aqueous reconstitution solution to the pharmaceutical composition and reconstituting the pharmaceutical composition.

In a fourth aspect there is provided a kit comprising the pharmaceutical composition of the first aspect or the vaccine of the second aspect, the kit comprising a first container comprising the pharmaceutical composition of the first aspect or the vaccine of the second aspect and a second container comprising a sterile aqueous reconstitution solution.

In a fifth aspect there is provided a method for producing the pharmaceutical composition of the first aspect, said method comprising
i) increasing the surface area to volume ratio of an aqueous composition comprising nucleic acid and lipid carrier particles,
ii) subjecting the aqueous composition to freezing conditions sufficient to freeze the aqueous composition, and
iii) drying the frozen composition.

In a sixth aspect there is provided a freeze-dried composition obtained from the method of the fifth aspect.

In a seventh aspect there is provided the use of the pharmaceutical composition of the first aspect, the vaccine of the second aspect or the kit of the fourth aspect in the manufacture of a medicament for treating a subject in need thereof.

In an eighth aspect there is provided the use of the pharmaceutical composition of the first aspect, the vaccine of the second aspect or the kit of the fourth aspect in the manufacture of a medicament for prophylaxis in a subject in need thereof.

In a ninth aspect there is provided a method for eliciting an immune response in a subject in need thereof comprising administering the pharmaceutical composition of the first aspect or the vaccine of the second aspect to the subject, optionally wherein the subject is a human subject.

In a tenth aspect there is provided the pharmaceutical composition of the first aspect or the vaccine of the second aspect for use in medicine.

In an eleventh aspect there is provided the pharmaceutical composition of the first aspect or the vaccine of the second aspect for use in the treatment or prevention of disease in a subject, optionally wherein the subject is a human subject.

### BRIEF DESCRIPTION OF THE FIGURES

FIG. 1: Impact of spin-freeze-drying related stresses on the self-amplifying mRNA (SAM) encapsulation efficiency, the total SAM content, hydrodynamic diameter of the lipid nanoparticles (LNPs) (Z-average) and the polydispersity of the lipid nanoparticles (LNPs).

FIG. 2: Schematic representation of the spin freeze drying apparatus. Vials were loaded in the glovebox (A) and were transferred, via the central load lock system with robotic arm (B), towards the spin freezing chamber (C). The spin-frozen vials were relocated, via the central load lock system with robotic arm (B), towards the drying chamber (D) that has eight temperature-controlled radiator pairs (indicated with numbers). Once fully dried, the spin freeze-dried vials were removed from the drying chamber towards the glovebox (A).

FIG. 3: Residual moisture content of the spin-freeze-dried cakes in function of the vial temperature during 90 minutes of secondary drying using the Karl Fisher method.

FIG. 4: Impact of secondary drying temperature on the CQA's of SAM-LNP: SAM encapsulated efficiency, the total SAM content, hydrodynamic diameter of the LNPs (Z-average) and the polydispersity of the LNPs.

FIG. 5: Analyzed CQA's of a mRNA vaccine to compare batch and spin-freeze-dried samples: SAM encapsulated efficiency, the total SAM content, hydrodynamic diameter (Z-average) and the polydispersity of the LNPs.

FIG. 6 for a graphical representation of vial temperatures over drying times for varying ramp rates for secondary drying and vial temperatures relative to collapse temperatures.

FIG. 7 shows that dehydration impacted each freezing-drying protocol in measures of mRNA encapsulation when compared to SAM-LNP vaccines pre-lyophilization (stored at −80° C.).

FIG. 8 shows that dehydration impacted each freezing-drying protocol in measures of hydrodynamic diameter (“LNP size (nm)” in the y-axis label) of the lipid nanoparticles when compared to SAM-LNP vaccines pre-lyophilization (stored at −80° C.).

FIG. 9 shows the residual moisture content of the products over 0, 5, 9, 13, 17, and 21 days of storage at 25° C.

FIG. 10 shows the percentage of baby hamster kidney (BHK) cells expressing eGFP after being transfected with the products, which had been reconstituted after 0, 5, and 9 days of storage (as lyophilized products) at 25° C. Lyophilizates were reconstituted using the same volume of nuclease-free water as before drying (643 μL), without correction of the residual moisture content still present in the cake.

FIG. 11 shows the effects of freeze-drying and stress from room temperature storage on LNP Z-average hydrodynamic diameter.

FIG. 12 shows the effects of freeze-drying and stress from room temperature storage on the LNP polydispersity index.

FIG. 13 shows the effects of freeze-drying and stress from room temperature storage on mRNA encapsulation.

FIG. 14 shows the effects of freeze-drying and stress from room temperature storage on mRNA integrity.

FIG. 15 shows that, generally, freeze-drying (i.e. across all freeze-drying conditions including spin freeze-drying, batch freeze-drying, and shelf-freeze drying) reduces the percentage of BHK cells expressing eGFP by about 30% when the measures are normalized to that of the non-lyophilized comparative stored at −80° C.—(see FIG. 15); however batch freeze-drying underperformed the other freeze-drying conditions. Those that were batch freeze-dried also underperformed the spin-lyophilized formats when incubated at 25° C. for each of the 4 weeks when evaluating the potency relative to that of the non-lyophilized conditions.

FIG. 16 shows that the drop in potency shown in FIG. 15 cannot be explained by initial dehydration, which reduces encapsulation efficiency by about 10%.

FIG. 17 shows that the drop in potency shown in FIG. 15 cannot be explained by initial dehydration, which increases LNP Z-average hydrodynamic diameter by about 10 nm.

FIG. 18 depicts the polydispersity index of the samples from FIGS. 15-17, which decreased over time in storage at 25° C.

FIG. 19 depicts how residual moisture content increases with longer storage at 25° C. in the samples from FIGS. 15-18.

### DETAILED DESCRIPTION OF THE INVENTION

Prior to setting forth the invention in detail, it may be helpful to the understanding of one of ordinary skill to define the following terms:

Unless otherwise explained or defined herein, all technical and scientific terms used herein have the same meaning as commonly understood by one of ordinary skill in the art to which this disclosure belongs. For example, definitions of common terms in molecular biology can be found in Benjamin Lewin, Genes V, published by Oxford University Press, 1994 (ISBN 0-19-854287-9); Kendrew et al. (eds.), The Encyclopaedia of Molecular Biology, published by Blackwell Science Ltd., 1994 (ISBN 0-632-02182-9); and Robert A. Meyers (ed.), Molecular Biology and Biotechnology: a Comprehensive Desk Reference, published by VCH Publishers, Inc., 1995 (ISBN 1-56081-569-8).

The articles “a” and “an” are used herein to refer to one or to more than one (i.e., to at least one) of the grammatical object of the article. By way of example, “an element” means one element or more than one element.

“Or” supports, contemplates, and when recited in the claims, claims “one or a combination of” as in “one or a combination of A, B, or C.” To illustrate, “A, B, or C” means A alone, B alone, C alone, the combination of A and B, the combination of A and C, the combination of B and C, and the combination of A, B, and C, unless otherwise illustrated. That is, “or” supports and contemplates “and” as in “and/or” wherein “and/or” includes any combinations within the list of alternatives without being limited solely to the combination of all alternatives in a list (i.e., “A, B, or C” includes “A and B” and is not limited to “A, B, and C”).

Furthermore, the recitation of a list of alternatives, which may be conjoined by “and” and from which at least one alternative is selected, further contemplates and supports all combinations within the list of alternatives. For example, “X is selected from the group of: A, B, and C” contemplates and supports “X is selected from the group of: A, B, C, and combinations thereof,” “X is selected from at least one of the group of: A, B, and C,” and “X is selected from one or more of the group of: A, B, and C.” For further example, “X is selected from the group consisting of A, B, and C” contemplates and supports “X is selected from the group consisting of A, B, C, and combinations thereof,” “X is selected from at least one of the group consisting of A, B, and C,” or “X is selected from one or more of the group consisting of A, B, and C.”

Each of the following contemplates and supports any of the others: “comprises,” “consists of,” “consists essentially of,” “is/are/being.” “is selected from,” “is at least selected from,” “is selected from the group of,” “is selected from the group consisting of,” “is at least selected from the group consisting of,” “is from at least one of the group consisting of,” and “is from one or more of the group consisting of.” For example and in consideration of the above regarding combinations of listed elements, recitation of “X comprises an A, a B, or a C” in the specification contemplates and supports embodiments wherein “X consists of an A, a B, or a C,” “X consists of an A, a B, a C, or combinations thereof,” “X consists of one or more of an A, a B, or a C,” “X is one or more of an A, a B, or a C,” “X is an A, a B, a C, or combinations thereof,” “X is selected from an A, a B, or a C,” “X is selected from an A, a B, a C, or combinations thereof,” “X is selected from the group consisting of an A, a B, a C, and combinations thereof,” “X is selected from at least one of the group consisting of an A, a B, and a C,” or “X is selected from one or more of the group consisting of an A, a B, and a C.”

When a specific component of an embodiment is listed—e.g. “X comprises A, B, or C”-then also supported and contemplated are any embodiments which specifically exclude any individual or combinations of components—e.g. “X comprises A, but not B or C” or “X comprises A but does not comprise B or C.”

“About” as used herein when referring to a measurable value such as an amount, a temporal duration, a quantum of measurement, and the like, is meant to encompass variations of +−20% or +−10%, for example +−5%, +−1%, +−0.1% from the specified value, as such variations are appropriate to perform the disclosed methods.

“Sequence,” “segment,” “nucleic acid,” or “region” as used within the context of a nucleic acid includes sense (i.e., positive) and anti-sense (i.e., negative, e.g. reverse complementary) sequences of the same nucleic acid. A “segment,” “sequence,” “nucleic acid,” or “region” that “encodes” a coding sequence, wherein the coding sequence is transcribed and/or translated, includes sense and antisense (e.g., reverse complementary) sequences of the same nucleic acid.

To illustrate how “sequence,” “segment.” “nucleic acid,” or “region” as used within the context of a nucleic acid includes sense (i.e. positive) and anti-sense, if a specific sequence, called “A”, is listed as having the sequence of 5′-ATGG-3′ in the sense strand (i.e. positive strand) then it is contemplated, supported, and when listed in the claims, claimed that A also has the sequence of 3′-TACC-5′ in the antisense strand (i.e. negative strand) or complementary strand (i.e. A comprises 5′-ATGG-3′ or 3′-TACC-5′).

“Sequence,” “region,” or “segment” as used herein, unless otherwise specified, also contemplates, and supports sequences incorporating different forms of nucleic acids, i.e., RNA and DNA, of the same information, or sequences incorporating differing nucleotides found in the different forms of the nucleic acids (i.e. uridines in RNA and thymidines in DNA), as well as sense and anti-sense (e.g. reverse complementary) information therein. To illustrate, if A in RNA (sense) is 5′-AUGG-3′, A also comprises 5′-ATGG-3′, being the sense DNA, and 3′-TACC-5′ being the anti-sense DNA, as well as 3′-UACC-5′, being the antisense RNA.

Amino acids refers to an amino acid selected from the group consisting of alanine (ala, A), arginine (arg, R), asparagine (asn, N), aspartic acid (asp, D), cysteine (cys, C),glutamine (gln, Q). glutamic acid (glu, E), glycine (gly, G), histidine (his, H), isoleucine (ile, I), leucine (leu, L), lysine (lys, K), methionine (met, M), phenylalanine (phe, F), proline (pro, P), serine (ser, S), threonine (thr, T), tryptophan (trp. W), tyrosine (tyr, Y), valine (val. V).

A “subject” as used herein is an animal, preferably a mammal, including humans, non-human primates, and non-primate mammals such as members of the rodent genus (including but not limited to mice and rats), the Cavia genus (including but not limited to guinea pigs) and members of the order Lagomorpha (including but not limited to rabbits). In an embodiment, the subject is a human.

“Buffer” refers to a buffered solution that resists changes in pH by the action of its acid-base conjugate components. The pH of the buffer will generally be chosen to stabilize the active material of choice. Generally, this will be in the range of physiological pH, although some proteins, can be stable at a wider range of pHs, for example acidic pH.

As used herein, “immune response” means the sequence of events occurring at the molecular, cellular or tissue level (i.e., at any level of biological organisation) in response to an antigen. In the context of the present disclosure, “immune response” may be the sequence of cellular (cell mediated) and/or humoral (antibody mediated) events occurring in response to an antigen (e.g., antigens on the surface of bacteria, viruses, fungi etc.) or in response to antigens that are translated from a nucleic acid that encodes said antigen. As used herein, “immunogenicity” means the ability of an antigen to elicit an immune response.

As used herein, “adjuvant” means a compound or substance (or combination of compounds or substances) that, when administered to a subject in conjunction with an antigen or antigens, for example as part of an immunogenic composition or vaccine, increases or enhances the subject's immune response to the administered antigen or antigens, compared to the immune response obtained in the absence of adjuvant. With respect to the present disclosure, the adjuvant may additionally mean a compound or substance (or combination of compounds or substances) that, when administered to a subject in conjunction with a pharmaceutical comprising nucleic acid and lipid carrier particles, for example as part of an immunogenic composition or vaccine, increases or enhances the subject's immune response to the protein (e.g. protein immunogen) encoded by the nucleic acid.

As used herein the term “immunogenic composition” relates to a composition of matter suitable for administration to a human or animal subject (e.g., in an experimental or clinical setting) that is capable of eliciting a specific immune response, e.g., against a pathogen. As such, an immunogenic composition includes one or more antigens (for example, polypeptide antigens) or antigenic epitopes. An immunogenic composition can also include one or more additional components capable of eliciting or enhancing an immune response, such as an excipient, carrier, and/or adjuvant. In certain instances, immunogenic compositions are administered to elicit an immune response that protects the subject, wholly or partially, against symptoms or conditions induced by a pathogen.

As used herein, “Pharmaceutical composition” refers to preparations which are in such a form as to permit the biological activity of the active ingredients to be unequivocally effective, and which contain no additional components which are toxic as administered to the subjects.

By “immunologically effective amount”, it is meant that the administration of that amount to an individual, either in a single dose or as part of a series, is effective for treatment, protection or prevention.

Administration of an immunologically effective amount elicits an immune response, including a protective immune response. This amount can vary depending upon the health and physical condition of the individual to be treated, age, the taxonomic group of individual to be treated (e.g. non-human primate, primate, etc.), the capacity of the individual's immune system to synthesise antibodies, the degree of protection desired, the formulation of the vaccine, the treating doctor's assessment of the medical situation, and other relevant factors. It is expected that the amount will fall in a relatively broad range.

As used herein “vaccine” refers to a composition that induces an immune response upon inoculation into a subject. In particular the term “vaccine” refers to a composition comprising a nucleic acid that encodes for an immunogen against which an immune response is induced upon inoculation of the composition into a subject. In some embodiments, the induced immune response provides protective immunity.

The “collapse temperature” or “Tcol” is the temperature at which the composition being dried softens to the point of not being able to support its own structure. The collapse temperature is the maximum temperature that the composition can withstand during primary drying without the composition collapsing. The collapse temperature can be determined using freeze drying microscopy.

The “glass transition temperature” or Tg′ refers to the temperature at which a composition changes from a glassy, amorphous or vitreous state to a rubbery state. Generally, Tg′ is determined using differential scanning calorimetry and is standardly taken as the temperature at which onset of the change of heat capacity (Cp) of the composition occurs upon scanning through the transition.

As used herein, the term “sublimation” refers to a process wherein materials change from a solid phase directly to a gaseous phase without passing through a liquid phase. With water, ice turns directly to water vapor without first melting to a liquid form, and then evaporating.

By “lyophilised” it is meant that a composition has been subjected to a “lyophilisation” or “freeze-drying” procedure which remove waters from the composition after the composition is frozen and placed under a vacuum. The lyophilisation or freeze-drying procedure allows ice that forms during freezing of the composition, to change directly from solid to vapor without passing through a liquid phase. The process consists of three separate, interdependent processes, freezing, primary drying (sublimation), and secondary drying (desorption). In an embodiment, the primary drying step may be referred to as the sublimation step. In an embodiment, the secondary drying step may be referred to as the desorption step.

In a first aspect there is provided a pharmaceutical composition said pharmaceutical composition comprising a nucleic acid and lipid carrier particles wherein the pharmaceutical composition is lyophilised and wherein either
(a) the percentage of nucleic acid that is encapsulated within the lipid carrier particles is greater than 75% of the total nucleic acid in the pharmaceutical composition; and/or
(b) the percentage of nucleic acid that remains encapsulated within the lipid carrier particles is reduced by less than 15% compared to the percentage of nucleic acid that is encapsulated within the lipid carrier particles in the pharmaceutical composition prior to lyophilisation.

(a) and (b) may be referred to as bullet (a) of the first aspect and bullet (b) of the first aspect respectively.

In an embodiment there is provided a pharmaceutical composition said pharmaceutical composition comprising a nucleic acid and lipid carrier particles wherein the pharmaceutical composition is lyophilised and wherein the percentage of nucleic acid that is encapsulated within the lipid carrier particles is greater than 75% of the total nucleic acid in the pharmaceutical composition.

In an embodiment there is provided a pharmaceutical composition said pharmaceutical composition comprising a nucleic acid and lipid carrier particles wherein the pharmaceutical composition is lyophilised and wherein the percentage of nucleic acid that remains encapsulated within the lipid carrier particles is reduced by less than 15% compared to the percentage of nucleic acid that is encapsulated within the lipid carrier particles in the pharmaceutical composition prior to lyophilisation.

In an embodiment there is provided a pharmaceutical composition said pharmaceutical composition comprising a nucleic acid and lipid carrier particles wherein the pharmaceutical composition is lyophilised and wherein the percentage of nucleic acid that is encapsulated within the lipid carrier particles is greater than 75% of the total nucleic acid in the pharmaceutical composition and the percentage of nucleic acid that remains encapsulated within the lipid carrier particles is reduced by less than 15% compared to the percentage of nucleic acid that is encapsulated within the lipid carrier particles in the pharmaceutical composition prior to lyophilisation.

The pharmaceutical composition of the present disclosure is thus lyophilised. It will however be understood by the skilled person that the pharmaceutical composition is lyophilised from an aqueous composition comprising nucleic acid and lipid carrier particles. Said aqueous composition may be referred to as the aqueous pre-lyophilisation composition, formulation, or solution. It will further be understood that the constituents of the aqueous composition are substantially present in the lyophilised pharmaceutical composition and at substantially the same concentration. Therefore, reference made herein to the composition of the aqueous composition (e.g., the aqueous composition comprises x, y, or z) is to be understood to refer also to the composition of the lyophilised pharmaceutical composition. (i.e., the aqueous composition and thus the lyophilised pharmaceutical composition comprises x, y, or z).

In an embodiment, the nucleic acid and lipid carrier particles are nucleic acid containing lipid carrier particles. In an embodiment the nucleic acid and lipid carrier particles are RNA containing lipid carrier particles. In an embodiment the nucleic acid and lipid carrier particles are RNA containing lipid nanoparticles (LNPs). Reference within the present disclosure to a nucleic acid (i.e., RNA) containing lipid carrier particle (i.e., LNP), means that the lipid carrier particle comprises an encapsulated RNA payload. In other words, it is meant that the nucleic acid containing lipid carrier particle comprise encapsulated nucleic acid, with the exception of any nucleic acid that has escaped from the carrier particle during the lyophilisation or freeze-drying procedure.

As described above, the encapsulation of nucleic acids within lipid carrier particles can be expressed in two ways.

Firstly, encapsulation of nucleic acids within lipid carrier particles can be expressed as a percentage compared to the total nucleic acid in the pharmaceutical composition (under bullet (a) in the first aspect). In an embodiment, the percentage of nucleic acid that is encapsulated within the lipid carrier particles is greater than 77.5%, greater than 80%, greater than 82.5%, greater than 85%, greater than 87.5%, greater than 90%, greater than 92.5%, greater than 95%, or greater than 97.5% compared to total nucleic acid in the pharmaceutical composition. In an embodiment, the percentage of nucleic acid that is encapsulated within the lipid carrier particles is between 75% and 97.5%, 75% and 95%, between 75% and 90%, or between 75% and 85% of the total nucleic acid in the pharmaceutical composition. As used herein “percentage of nucleic acid that is encapsulated within the lipid carrier particles is greater than 75% of the total nucleic acid in the pharmaceutical composition” may also be referred to as “greater than 75% of the total nucleic acid in the pharmaceutical composition is encapsulated within the lipid carrier particles”.

Secondly, encapsulation of nucleic acid within the lipid carrier particles can be expressed as percentage loss compared to the percentage of nucleic acid that is encapsulated within the lipid carrier particles in the pharmaceutical composition prior to lyophilisation (under bullet (b) in the first aspect). Percentage loss is determined by measuring percentage encapsulation both prior to lyophilisation and following lyophilisation and then calculating the percentage loss in encapsulation between the pre- and post-lyophilised pharmaceutical compositions. The pre-lyophilised composition is the final aqueous composition formulated prior to lyophilisation. The pre-lyophilised composition may be referred to as the Final Bulk (FB) or the aqueous composition. In an embodiment the pre-lyophilised composition may be referred to as the aqueous pre-lyophilisation solution. In an embodiment, the percentage of nucleic acid that remains encapsulated within the lipid carrier particles is reduced by less than 12.5%, less than 10%, less than 7.5%, less than 5% or less than 2.5% compared to the percentage of nucleic acid that is encapsulated within the lipid carrier particles in the pharmaceutical composition prior to lyophilisation. In an embodiment, the percentage of nucleic acid that remains encapsulated within the lipid carrier particles is reduced by between 2.5% and 15%, 5% and 15%, between 7.5% and 15%, or by between 7.5% and 12.5% compared to the percentage of nucleic acid that is encapsulated within the lipid carrier particles in the pharmaceutical composition prior to lyophilisation. In an embodiment the pharmaceutical composition prior to lyophilisation is the final bulk. In an embodiment the pharmaceutical composition prior to lyophilisation is the aqueous composition. As referred to herein “the percentage of nucleic acid that remains encapsulated within the lipid carrier particles is reduced by less than 15% compared to the percentage of nucleic acid that is encapsulated within the lipid carrier particles in the pharmaceutical composition prior to lyophilisation” may be referred to as “the percentage of nucleic acid that remains encapsulated within the lipid carrier particles does not decrease by more than 15% compared to the percentage of nucleic acid that is encapsulated within the lipid carrier particles in the pharmaceutical composition prior to lyophilisation”.

Alternatively, bullet (b) in the first aspect may be expressed as the percentage of nucleic acid that remains encapsulated within lipid carrier particles following lyophilisation, compared to the percentage of nucleic acid that was encapsulated within lipid carrier particles pre-lyophilisation (i.e., wherein the percentage of nucleic acid that was encapsulated within lipid carrier particles pre-lyophilisation is considered 100%). For example, in an embodiment, the percentage of nucleic acid that remains encapsulated within the lipid carrier particles following lyophilisation is at least 85%, at least 87.5%, at least 90%, at least 92.5%, at least 95%, or at least 97.5% of the percentage of nucleic acid that was encapsulated within the lipid carrier particles pre-lyophilisation. As such, in an embodiment there is provided a pharmaceutical composition said pharmaceutical composition comprising a nucleic acid and lipid carrier particles wherein the pharmaceutical composition is lyophilised and wherein the percentage of nucleic acid that is encapsulated within the lipid carrier particles is greater than 75% of the total nucleic acid in the pharmaceutical composition and/or the percentage of nucleic acid that remains encapsulated within the lipid carrier particles following lyophilisation is at least 85%, at least 87.5%, at least 90%, at least 92.5%, at least 95%, or at least 97.5% compared to the percentage of nucleic acid that was encapsulated within the lipid carrier particles pre-lyophilisation.

In an embodiment, the lipid carrier particles encapsulate a nucleic acid payload, for example an RNA payload.

In an embodiment the nucleic acid and lipid carrier particles are nucleic acid containing lipid carrier particles. In an embodiment the nucleic acid and lipid carrier particles are mRNA containing lipid carrier particles.

In an embodiment the percentage of nucleic acid that is encapsulated within the lipid carrier particles is defined according to encapsulation efficiency. Thus, in an embodiment, there is provided a pharmaceutical composition said pharmaceutical composition comprising a nucleic acid and lipid carrier particles wherein the pharmaceutical composition is lyophilised and wherein either
(a) the encapsulation efficiency is greater than 75%, greater than 77.5%, greater than 80%, greater than 82.5%, greater than 85%, greater than 87.5%, greater than 90%, greater than 92.5%, greater than 95%, or greater than 97.5%; and/or
(b) the encapsulation efficiency is reduced by less than 15%, less than 12.5%, less than 10%, less than 7.5%, less than 5%, or less than 2.5% compared to the encapsulation efficiency of the pharmaceutical composition prior to lyophilisation.

In an embodiment, the lyophilised pharmaceutical composition comprises a polydispersity index of less than 0.8, less than 0.5 or less than 0.2 as assessed by dynamic light scattering (DLS) analysis. In an embodiment the lyophilised pharmaceutical composition comprises a PDI of between 0.1 and 0.25 (for example between 0.1 and 0.2) as assessed by DLS analysis. The polydispersity index (PDI), a measure of size-based heterogeneity in a pharmaceutical composition, for example the heterogeneity in lipid particle distribution. Measurement of PDI via DLS analysis can be conducted for example using a Malvern Nano ZS Zetasizer (e.g., essentially as described in Muramatsu et al 2022, Mol Ther; 30 (5): 1941-1951).

In an embodiment the percentage of nucleic acid that remains encapsulated within the lipid carrier particles and/or the PDI is assessed following reconstitution of the lyophilised pharmaceutical composition. In an embodiment the percentage of nucleic acid that remains encapsulated within the lipid carrier particles and/or the PDI is assessed within 2 hours of reconstitution. In an embodiment, the percentage of nucleic acid that remains encapsulated within the lipid carrier particles and/or the PDI is assessed in an aliquot of the pharmaceutical composition, said aliquot being frozen at less than −20° C. (e.g., frozen at approximately −80° C.) until the assessment(s) are conducted.

In an embodiment the lyophilised pharmaceutical composition is reconstituted with any medium comprising a tonicity agent (e.g., sucrose) wherein said medium is safe for administration to a subject, taking into consideration the route of administration. In an embodiment, the lyophilised pharmaceutical composition is reconstituted with sterile water, saline, or liquid adjuvant. The precise composition of liquid used for reconstitution (i.e., diluent) will depend on both the contents of a composition being reconstituted and the subsequent use of the reconstituted contents. It is however envisaged that liquid used for reconstitution (i.e., diluent) is at an acceptable and physiologically relevant pH, temperature and osmolarity such that the liquid used for reconstitution doesn't itself impact the percentage of nucleic acid that remains encapsulated within the lipid carrier particles and/or the PDI. Thus, in an embodiment, the lyophilised pharmaceutical composition is reconstituted with a diluent, wherein the diluent has no impact on percentage encapsulation or PDI. In an embodiment the lyophilised pharmaceutical composition is reconstituted with sterile water. In an embodiment, the lyophilised pharmaceutical composition is reconstituted with saline, optionally wherein said saline is at a concentration of 0.4%±0.1% sodium chloride or 0.9%±0.1% sodium chloride.

In an embodiment, the percentage of the nucleic acid that is encapsulated within the lipid carrier particles is determined by comparing the signal of a nucleic acid-binding fluorescent dye in the absence and presence of a detergent, wherein in the absence of a detergent, the signal comes only from free (unencapsulated) nucleic acid and wherein in the presence of a detergent, the lipid carrier particle is disrupted so that the signal comes from the total nucleic acid (both encapsulated and non-encapsulated). In an embodiment the percentage of the nucleic acid that is encapsulated within the lipid carrier particles is calculated by firstly extrapolating the concentration of free and total nucleic acid from a standard curve, secondly expressing the concentration of free nucleic acid as a percentage of the concentration of total nucleic acid and thirdly subtracting the percentage free nucleic acid from the percentage total nucleic acid (i.e. subtracting the percentage unencapsulated nucleic acid from 100%). In an embodiment said standard curve plots the signal of the nucleic acid binding fluorescent dye against nucleic acid standards of known concentration. In other words, the signal of the nucleic acid binding fluorescent dye can be compared to a standard curve generated using nucleic acid samples of known concentration to extrapolate the concentration of both the free (unencapsulated) nucleic acid and the total nucleic acid (both encapsulated and non-encapsulated).

In an embodiment the percentage of the nucleic acid that is encapsulated within the lipid carrier particles is determined post-lyophilisation to calculate the percentage of nucleic acid that is encapsulated within the lipid carrier particles, compared to the total nucleic acid in the pharmaceutical composition. In an embodiment the percentage of the nucleic acid that is encapsulated within the lipid carrier particles determined both prior to (or pre-) lyophilisation (e.g. in the Final Bulk) and post-lyophilisation in order to calculate the percentage reduction in the percentage of nucleic acid encapsulated within the lipid carrier particles post-lyophilisation compared to the percentage of nucleic acid that is encapsulated within the lipid carrier particles in the pharmaceutical composition prior to (or pre-) lyophilisation.

In an embodiment, the nucleic acid-binding dye is Ribogreen. In an embodiment the nucleic acid binding dye is provided within the Ribogreen reagent. In an embodiment, the detergent is Triton X-100, optionally at a concentration of between 0.01% and 1% (w/v) e.g., between 0.05% and 0.5% (w/v). In an embodiment, the detergent is Triton X-100 at a concentration of around 0.1% (w/v). Thus, in an embodiment, the percentage of the nucleic acid that is encapsulated within the lipid carrier particles is determined by comparing the signal of Ribogreen in the absence and presence of Triton X-100 at a concentration of around 0.1% (w/v). The percentage of the nucleic acid that is encapsulated within the lipid carrier particles is calculated by firstly extrapolating the concentration of free and total nucleic acid from a standard curve, secondly expressing the concentration of free nucleic acid as a percentage of the concentration of total nucleic acid and thirdly subtracting the percentage free nucleic acid from the percentage total nucleic acid (i.e. subtracting the percentage unencapsulated nucleic acid from 100%), wherein said standard curve plots the signal of the nucleic acid binding fluorescent dye against nucleic acid standards of known concentration. Unless stated to the contrary, percentage of the nucleic acid that is encapsulated within the lipid carrier particles is determined in accordance with this embodiment.

In an embodiment the pharmaceutical composition of the first aspect is lyophilised to form a cylindrical shaped cake.

In an embodiment the pharmaceutical composition of the first aspect is contained within a vessel. In an embodiment, the vessel is a vial, optionally with a diameter of approximately 10-40 mm. In an embodiment, the vial used is substantially cylindrically shaped and/or comprises a substantially cylindrically shaped inner circumferential wall.

In an embodiment, the lyophilised pharmaceutical composition is coated in a layer of substantially uniform thickness against an inner surface of the circumferential wall of the vessel. By substantially uniform thickness it is meant that the thickness of the lyophilised pharmaceutical composition varies less than about 10% of the average thickness from the upper to the lower end of the vessel, providing that the thickness is measured as the average between any surface peaks or troughs which may result from fluid dynamics during the freezing process. The lyophilised pharmaceutical composition is stretched out as a relatively thin film or shell over the inner surface of the circumferential wall of the vessel.

In an embodiment, the entire volume of the lyophilised pharmaceutical composition is coated in a layer of substantially uniform thickness against the inner surface of the circumferential wall of the vessel. In an embodiment greater than 80%, greater than 82.5%, greater than 85%, greater than 87.5%, greater than 90%, greater than 92.5%, greater than 95% or greater than 97.5% of the lyophilised pharmaceutical composition is coated in a layer of substantially uniform thickness against the inner surface of the circumferential wall of the vessel. The percentage of the lyophilised pharmaceutical composition that is coated to the inner surface of the circumferential wall of the vessel is calculated by measuring the covered surface area and the layer thickness.

A typical thickness of the layer is about 1 mm e.g., 0.5 to 2 mm, 0.7 to 1.5 mm, or 0.75 to 1.25 mm. In an embodiment the layer of substantially uniform thickness is between 0.5 mm and 2.5 mm.

In an embodiment, the lyophilised pharmaceutical composition is not substantially coated on a bottom part of the vessel(s). By bottom part of the vessel(s) it is meant that a base or floor portion of the vessel is substantially free from the lyophilised pharmaceutical composition. This is because of axial rotation of the vessel during the process of freezing the pharmaceutical composition results in a lyophilizate that is coated in a layer of substantially uniform thickness against an inner surface of the circumferential wall of the vessel. As a result, a bottom part of the vessel is substantially free from the lyophilised pharmaceutical composition. In an embodiment, a bottom part of the vessel is substantially free of lyophilised cake.

The nucleic acid of the present disclosure may be DNA or RNA (including hybrids thereof) but is preferably RNA.

Thus, in an embodiment, the pharmaceutical composition comprises RNA and lipid carrier particles. As such there is provided a pharmaceutical composition said pharmaceutical composition comprising RNA and lipid carrier particles wherein the pharmaceutical composition is lyophilised and wherein either (a) the percentage of RNA that is encapsulated within the lipid carrier particles is greater than 75% of the total RNA in the pharmaceutical composition; and/or (b) the percentage of RNA that remains encapsulated within the lipid carrier particles is reduced by less than 15% compared to the percentage of RNA that is encapsulated within the lipid carrier particles in the pharmaceutical composition prior to lyophilisation.

In an embodiment the RNA is present at a concentration of between 5-250 μg/ml. 10-100 μg/ml. 25-100 μg/ml, 25-75 μg/ml, or 50-75 μg/ml. In an embodiment the RNA is present at a concentration of 60 μg/ml. In an embodiment the RNA is provided in the pharmaceutical composition at a concentration of between 5-250 μg/ml. 10-100 μg/ml. 25-100 μg/ml. 25-75 μg/ml, or 50-75 μg/ml, optionally 60 μg/ml.

In a preferred embodiment, the RNA is messenger RNA (mRNA).

In an embodiment the mRNA is an artificial (or recombinant) ribonucleic acid encoding at least one protein, optionally wherein the protein is a protein immunogen, which may be translated in a cell (i.e., mRNA). In an embodiment, the mRNA is neither, nor comprised within, a viral vector or virus-based vaccine (such as a live-attenuated virus vaccine).

In an embodiment, the mRNA will comprise, in the 5′ to 3′ direction: 5′ Cap. 5′ UTR, open reading frame encoding at least one protein (optionally a protein immunogen). 3′UTR. and 3′ poly-A tail (in particular, the 5′ Caps: 5′ UTRs. 3′UTRs and 3′ poly-A tails as detailed elsewhere herein).

Thus, in an embodiment the pharmaceutical composition comprises mRNA and lipid carrier particles. As such there is provided a pharmaceutical composition said pharmaceutical composition comprising mRNA and lipid carrier particles wherein the pharmaceutical composition is lyophilised and wherein either (a) the percentage of mRNA that is encapsulated within the lipid carrier particles is greater than 75% of the total mRNA in the pharmaceutical composition; and/or (b) the percentage of mRNA that remains encapsulated within the lipid carrier particles is reduced by less than 15% compared to the percentage of mRNA that is encapsulated within the lipid carrier particles in the pharmaceutical composition prior to lyophilisation.

The mRNA molecules can have various lengths but are typically 500-25,000 ribonucleotides long e.g., 1000-25,000, 1000-20,000, 1000-15,000, 1000-10,000, 1000-5000, 1000-3000, 1000-2500, 1000-2500 or 1000-2000 ribonucleotides long.

In an embodiment, the mRNA is less than 25,000 ribonucleotides in length but is greater than 2000 ribonucleotides in length, greater than 2500 ribonucleotides in length, greater than 3000 ribonucleotides in length, greater than 3500 ribonucleotides in length, greater than 4000 ribonucleotides in length, greater than 4500 ribonucleotides in length, greater than 5000 ribonucleotides in length, greater than 5500 ribonucleotides in length, greater than 6000 ribonucleotides in length, greater than 6500 ribonucleotides in length, greater than 7000 ribonucleotides in length, greater than 7500 ribonucleotides in length or greater than 8000 ribonucleotides in length. In an embodiment the mRNA less than 15,000 ribonucleotides in length but is greater than 4500 ribonucleotides in length e.g. greater than 5000 ribonucleotides in length.

In a particular embodiment, there is therefore provided a pharmaceutical composition said pharmaceutical composition comprising mRNA and lipid carrier particles wherein the pharmaceutical composition is lyophilised and wherein either (a) the percentage of mRNA that is encapsulated within the lipid carrier particles is greater than 75% of the total mRNA in the pharmaceutical composition; and/or (b) the percentage of mRNA that remains encapsulated within the lipid carrier particles is reduced by less than 15% compared to the percentage of mRNA that is encapsulated within the lipid carrier particles in the pharmaceutical composition prior to lyophilisation, wherein the mRNA is less than 12,500 ribonucleotides in length but is greater than 2500 ribonucleotides, greater than 3000 ribonucleotides, greater than 3500 ribonucleotides, greater than 4000 ribonucleotides, greater than 4500 ribonucleotides or greater than 5000 ribonucleotides in length.

In some embodiments, the mRNA is self-amplifying and may be referred to as self-amplifying mRNA (SAM). As used herein, the terms self-amplifying mRNA and self-replicating mRNA may be used interchangeably. Where the mRNA is self-amplifying, it is generally larger and typically contains the basic elements described above (a cap, 5′ UTR, 3′ UTR, and poly (A) tail of variable length) but will have a large open reading frame (ORF) at the 5′ end that may encode (i) an RNA-dependent RNA polymerase which can transcribe RNA from the self-amplifying mRNA and (ii) at least one heterologous protein, optionally a protein immunogen.

A self-amplifying mRNA molecule is typically a positive-strand molecule which can be directly translated after delivery to a cell, and this translation provides an RNA-dependent RNA polymerase which then produces both antisense and sense transcripts from the delivered RNA. Thus, the delivered RNA leads to the production of multiple daughter RNAs. These daughter RNAs, as well as collinear subgenomic transcripts, may be translated themselves to provide in situ expression of the encoded protein or may be transcribed to provide further transcripts with the same sense as the delivered RNA, which are translated to provide in situ expression of the encoded protein. The overall result of this sequence of transcriptions is substantial amplification in the number of the introduced RNAs, and so the encoded protein (potentially in addition to further proteins as detailed above) becomes a major polypeptide product of the cells.

Self-amplifying mRNA can be produced using replication elements derived from, e.g., alphaviruses, and substituting sequences encoding the structural viral proteins with that encoding at least one heterologous protein. The polymerase can be an alphavirus replicase e.g., comprising one or more of alphavirus proteins nsP1, nsP2, nsP3 and nsP4. Or, such alphavirus-based self-amplifying mRNA can use a replicase from, for example, a Sindbis virus, a Semliki forest virus, an eastern equine encephalitis virus (EEEV), or a Venezuelan equine encephalitis virus (VEEV). Mutant or wild-type virus sequences can be used e.g., the attenuated TC83 mutant of VEEV has been used for self-amplifying RNA (see WO 2005/113782). Thus, a typical self-amplifying mRNA encodes at least one protein and may have two open reading frames. The first (5′) open reading frame encodes a replicase, in particular an alphavirus replicase (e.g., as detailed above); the second (3′) open reading frame encodes the at least one protein (optionally a protein immunogen). Further open reading frames may also be present, encoding (i) one or more further proteins and/or (ii) accessory polypeptides.

Self-amplifying mRNA may alternatively comprise separate RNA molecules: (i) a RNA construct for expressing an RNA-dependent RNA polymerase and (ii) a RNA replicon that can be replicated by the replicase in trans, wherein the RNA construct for expressing alphavirus replicase comprises a 5′-cap for driving translation of the replicase. Generally, the RNA will comprise, in the 5′ to 3′ direction: 5′ Cap, 5′ UTR, open reading frame encoding at least one protein (optionally a protein immunogen), 3′UTR, and 3′ poly-A tail (in particular, the 5′ Caps: 5′ UTRs, 3′UTRs and 3′ poly-A tails as detailed above throughout this subsection).

Thus, in a particular embodiment the pharmaceutical composition comprises mRNA and lipid carrier particles, wherein said mRNA is self-amplifying mRNA. As such there is provided a pharmaceutical composition said pharmaceutical composition comprising self-amplifying mRNA (SAM) and lipid carrier particles wherein the pharmaceutical composition is lyophilised and wherein either (a) the percentage of SAM that is encapsulated within the lipid carrier particles is greater than 75% of the total SAM in the pharmaceutical composition; and/or (b) the percentage of SAM that remains encapsulated within the lipid carrier particles is reduced by less than 15% compared to the percentage of SAM that is encapsulated within the lipid carrier particles in the pharmaceutical composition prior to lyophilisation.

SAM molecules can have various lengths, but are typically 5000 to 25000 ribonucleotides long, such as 8000 to 15000 ribonucleotides long, for example 9000 to 12000 ribonucleotides long.

The nucleic acid of the present disclosure is, in particular, provided in purified or substantially purified form; that is, substantially free from other nucleic acids (e.g., free or substantially free from naturally occurring nucleic acids, such as further nucleic acids expressed by a host cell) or from other proteins or cellular debris. Said nucleic acids is generally at least 50% pure (by weight), such as at least 60%, 70%, 80%, 90%, or 95% pure (by weight).

The nucleic acid may be for the expression of at least one protein in vitro from a host cell (i.e., the nucleic acid is, or is part of, an expression vector). Suitable nucleic acid expression vectors (in particular, DNA expression vectors) can comprise, for example, (1) an origin of replication; (2) a selectable marker gene; (3) one or more expression control elements, such as a transcriptional control element (e.g., a promoter, an enhancer, or a terminator), and/or one or more translation signals; and (4) a signal sequence or leader sequence for targeting to the secretory pathway in a selected host cell. However, in an alternative embodiment, the nucleic acid is for the expression of at least one protein in vivo in a subject (i.e., the nucleic acid is, or is part of, a nucleic acid-based vaccine). In such preferred embodiments, in addition to a sequence encoding the at least one protein, the nucleic acid may comprise one or more heterologous sequences, such as a sequence encoding a further protein (e.g., as detailed below) and/or a control sequence, in particular a promoter or an internal ribosome entry site.

In an embodiment the nucleic acid (e.g. mRNA) encodes at least one protein, optionally a protein immunogen. The nucleic acid (e.g., mRNA) may encode a single protein. Said protein may be an immunogen i.e., a protein that elicits an immune response against a bacterium, a virus, a fungus, or a parasite (or, in some embodiments, against an allergen; and in other embodiments, against a tumor antigen). After administration of the nucleic acid the immunogen is translated in vivo and can elicit an immune response in the recipient. The immune response may comprise an antibody response (usually including IgG) and/or a cell-mediated immune response. The protein immunogen will typically elicit an immune response which recognises the corresponding bacterial, viral, fungal or parasite (or allergen or tumour) polypeptide, but in some embodiments the polypeptide may act as a mimotope to elicit an immune response which recognises a bacterial, viral, fungal or parasite saccharide. The immunogen will typically be a surface polypeptide e.g., an adhesin, a hemagglutinin, an envelope glycoprotein, a spike glycoprotein, etc.

It will be understood that the present disclosure is not limited by the protein that is encoded by the nucleic acid. However, without limiting the disclosure in any way, in some embodiments the protein elicits an immune response against one of these bacteria:
Neisseria meningitidis: useful immunogens include, but are not limited to, membrane proteins such as adhesins, autotransporters, toxins, iron acquisition proteins, and factor H binding protein. A combination of three useful polypeptides is disclosed in reference (Giuliani et al. (2006) Proc Natl Acad Sci USA 103 (29): 10834-9.)
Streptococcus pneumoniae: useful polypeptide immunogens are disclosed in WO2009/016515. These include, but are not limited to, the RrgB pilus subunit, the beta-N-acetyl-hexosaminidase precursor (spr0057), spr0096, General stress protein GSP-781 (spr2021, SP2216), serine/threonine kinase StkP (SP1732), and pneumococcal surface adhesin PsaA.
Streptococcus pyogenes: useful immunogens include, but are not limited to, the polypeptides disclosed in WO02/34771 and WO2005/032582.
Moraxella catarrhalis.
Bordetella pertussis: Useful pertussis immunogens include, but are not limited to, pertussis toxin or toxoid (PT), filamentous haemagglutinin (FHA), pertactin, and agglutinogens 2 and 3.
Staphylococcus aureus: Useful immunogens include, but are not limited to, the polypeptides disclosed in WO2010/119343, such as a hemolysin, esxA, esxB, ferrichrome-binding protein (sta006) and/or the sta011 lipoprotein.
Clostridium tetani: the typical immunogen is tetanus toxoid.
Cornynebacterium diphtheriae: the typical immunogen is diphtheria toxoid.
Haemophilus influenzae: Useful immunogens include, but are not limited to, the polypeptides disclosed in WO2006/110413 and WO2005/111066.
Pseudomonas aeruginosa
Streptococcus agalactiae: useful immunogens include, but are not limited to, the polypeptides disclosed in WO2009/016515.
Chlamydia trachomatis: Useful immunogens include, but are not limited to, MOMP, PepA, LcrE (e.g., as disclosed in WO2006/138004), ArtJ, DnaK, CT398, OmpH-like, L7/L12, OmcA, AtoS, CT547, Eno, HtrA (e.g., as disclosed in WO2009/109860) and MurG (e.g. as disclosed in WO2005/002619).
Chlamydia pneumoniae: Useful immunogens include, but are not limited to, the polypeptides disclosed in WO02/02606.
Helicobacter pylori: Useful immunogens include, but are not limited to, CagA, VacA, NAP, and/or urease (WO03/018054).
Escherichia coli: Useful immunogens include, but are not limited to, immunogens derived from enterotoxigenic E. coli (ETEC), enteroaggregative E. coli (EAggEC), diffusely adhering E. coli (DAEC), enteropathogenic E. coli (EPEC), extraintestinal pathogenic E. coli (ExPEC) and/or enterohemorrhagic E. coli (EHEC). ExPEC strains include uropathogenic E. coli (UPEC) and meningitis/sepsis-associated E. coli (MNEC). Useful UPEC polypeptide immunogens are disclosed in WO2006/091517 and WO2008/020330. Useful MNEC immunogens are disclosed in WO2006/089264. A useful immunogen for several E. coli types is AcfD (WO2009/104092).
Bacillus anthracis
Yersinia pestis: Useful immunogens include, but are not limited to, those disclosed in WO2009/031043 and WO2007/049155.
Staphylococcus epidermis
Clostridium perfringens or Clostridium botulinums
Legionella pneumophila
Coxiella burnetii
Brucella, such as B. abortus, B. canis, B. melitensis, B. neotomae, B. ovis, B. suis, B. pinnipediae.
Francisella, such as F. novicida, F. philomiragia, F. tularensis.
Neisseria gonorrhoea.
Treponema pallidum
Haemophilus ducreyi
Enterococcus faecalis or Enterococcus faecium
Staphylococcus saprophyticus
Yersinia enterocolitica
Mycobacterium tuberculosis
Rickettsia
Listeria monocytogenes
Vibrio cholerae
Salmonella typhi
Borrelia burgdorferi
Porphyromonas gingivalis
Klebsiella

It will be understood that the present disclosure is not limited by the protein that is encoded by the nucleic acid. However, without limiting the disclosure in any way, in some embodiments the protein elicits an immune response against one of these viruses:
Orthomyxovirus: Useful immunogens can be from an influenza A, B or C virus, such as the hemagglutinin, neuraminidase or matrix M2 proteins. Where the immunogen is an influenza A virus hemagglutinin it may be from any subtype e.g., H1, H2, H3, H4, H5, H6, H7, H8, H9, H10, H11, H12, H13, H14, H15 or H16.
Paramyxoviridae viruses: Viral immunogens include, but are not limited to, those derived from Pneumoviruses (e.g., respiratory syncytial virus, RSV), Rubulaviruses (e.g. mumps virus), Paramyxoviruses (e.g. parainfluenza virus), Metapneumoviruses and Morbilliviruses (e.g. measles).
Poxviridae: Viral immunogens include, but are not limited to, those derived from Orthopoxvirus such as Variola vera, including but not limited to, Variola major and Variola minor.
Picornavirus: Viral immunogens include, but are not limited to, those derived from Picornaviruses, such as Enteroviruses, Rhinoviruses, Heparnavirus, Cardioviruses and Aphthoviruses. In one embodiment, the enterovirus is a poliovirus e.g., a type 1, type 2 and/or type 3 poliovirus. In another embodiment, the enterovirus is an EV71 enterovirus. In another embodiment, the enterovirus is a coxsackie A or B virus.
Bunyavirus: Viral immunogens include, but are not limited to, those derived from an Orthobunyavirus, such as California encephalitis virus, a Phlebovirus, such as Rift Valley Fever virus, or a Nairovirus, such as Crimean-Congo hemorrhagic fever virus.
Heparnavirus: Viral immunogens include, but are not limited to, those derived from a Heparnavirus, such as hepatitis A virus (HAV).
Filovirus: Viral immunogens include, but are not limited to, those derived from a filovirus, such as an Ebola virus (including a Zaire, Ivory Coast, Reston, or Sudan ebolavirus) or a Marburg virus.
Togavirus: Viral immunogens include, but are not limited to, those derived from a Togavirus, such as a Rubivirus, an Alphavirus, or an Arterivirus. This includes rubella virus.
Flavivirus: Viral immunogens include, but are not limited to, those derived from a Flavivirus, such as Tick-borne encephalitis (TBE) virus, Dengue (types 1, 2, 3 or 4) virus, Yellow Fever virus, Japanese encephalitis virus, Kyasanur Forest Virus, West Nile encephalitis virus, St. Louis encephalitis virus, Russian spring-summer encephalitis virus, Powassan encephalitis virus.
Pestivirus: Viral immunogens include, but are not limited to, those derived from a Pestivirus, such as Bovine viral diarrhea (BVDV), Classical swine fever (CSFV) or Border disease (BDV).
Hepadnavirus: Viral immunogens include, but are not limited to, those derived from a Hepadnavirus, such as Hepatitis B virus. A composition can include hepatitis B virus surface antigen (HBsAg).
Other hepatitis viruses: A composition can include an immunogen from a hepatitis C virus, delta hepatitis virus, hepatitis E virus, or hepatitis G virus.
Rhabdovirus: Viral immunogens include, but are not limited to, those derived from a Rhabdovirus, such as a Lyssavirus (e.g., a Rabies virus) and Vesiculovirus (VSV).
Caliciviridae: Viral immunogens include, but are not limited to, those derived from Calciviridae, such as Norwalk virus (Norovirus), and Norwalk-like Viruses, such as Hawaii Virus and Snow Mountain Virus.
Coronavirus: Viral immunogens include, but are not limited to, those derived from a SARS coronavirus (e.g., SARS-COV-2), avian infectious bronchitis (IBV), Mouse hepatitis virus (MHV), and Porcine transmissible gastroenteritis virus (TGEV). The coronavirus immunogen may be a spike polypeptide. Useful Coronavirus antigens (e.g., SARS-COV-2 antigens) include the spike, M, E, HE, Nuclocapsid, Plpro and 3CLPro proteins, in particular spike protein. In an embodiment, the Coronavirus antigen is a SARS-COV-2 spike protein. Said SARS-COV-2 spike protein may be from any variant, e.g., Omicron (such as Omicron BA.1, BA.2, BA3, BA.4 or BA.5), Alpha, Epsilon, Eta, Theta, Kappa, Iota, Zeta, Mu, Lambda, Beta, Gamma, or Delta. In an embodiment, said SARS-COV-2 spike protein includes one or more mutations relative to the wild-type protein, in particular one or more (e.g., two) mutations to proline resides. Said one or more mutations may be introduced to stabilize said SARS-COV-2 spike protein in its pre-fusion conformation.
Retrovirus: Viral immunogens include, but are not limited to, those derived from an Oncovirus, a Lentivirus (e.g. HIV-1 or HIV-2) or a Spumavirus.
Reovirus: Viral immunogens include, but are not limited to, those derived from an Orthoreovirus, a Rotavirus, an Orbivirus, or a Coltivirus.
Parvovirus: Viral immunogens include, but are not limited to, those derived from Parvovirus B19.
Herpesvirus: Viral immunogens include, but are not limited to, those derived from a human herpesvirus, such as, by way of example only, Herpes Simplex Viruses (HSV) (e.g., HSV types 1 and 2), Varicella-zoster virus (VZV), Epstein-Barr virus (EBV), Cytomegalovirus (CMV), Human Herpesvirus 6 (HHV6), Human Herpesvirus 7 (HHV7), and Human Herpesvirus 8 (HHV8).
Papovaviruses: Viral immunogens include, but are not limited to, those derived from Papillomaviruses and Polyomaviruses. The (human) papillomavirus may be of serotype 1, 2, 4, 5, 6, 8, 11, 13, 16, 18, 31, 33, 35, 39, 41, 42, 47, 51, 57, 58, 63 or 65 e.g. from one or more of serotypes 6, 11, 16 and/or 18.
Adenovirus: Viral immunogens include those derived from adenovirus serotype 36 (Ad-36).

As described above, the nucleic acid (e.g., RNA) may encode a single protein or alternatively, the nucleic acid may encode multiple proteins. Multiple proteins (e.g., multiple immunogens) can be presented as a single polypeptide (fusion polypeptide) or as separate polypeptides. If proteins are expressed as separate polypeptides, then one or more of these may be provided with an upstream internal ribosome entry site (IRES) or an additional viral promoter element. Alternatively, multiple proteins may be expressed from a polyprotein that encodes individual proteins fused to a short autocatalytic protease or as inteins.

In an embodiment, the nucleic acid of the present disclosure encodes a primary immunogen and at least one further protein. The at least one further protein may be a nanoparticle, e.g. a ferritin nanoparticle (e.g. which is encoded, along with the primary immunogen, by a single open reading frame, resulting in expression of a single polypeptide). In embodiments, the at least one further protein is an antigen; and as such may comprise, or may be, a viral, bacterial, fungal, parasitic, tumour, or allergenic (i.e., from, or derived from, an allergen) antigen; typically encoded by a separate open reading frame to the primary immunogen. The at least one further protein will typically be a pathogen antigen. The at least one further protein will typically be an antigen that is a surface polypeptide e.g., a spike glycoprotein, a haemagglutinin, an adhesin or an envelope glycoprotein. In a particular embodiment, the at least one further protein is an antigen from, or derived from, a virus, in particular a virus causing respiratory disease, in particular a Coronavirus.

In an embodiment the nucleic acid of the present disclosure is a plurality of mRNA polynucleotides said mRNA polynucleotides encoding different proteins. In an embodiment said plurality of mRNA polynucleotides are mixed prior to encapsulation within the lipid carrier particles (e.g., within lipid nanoparticles). In an embodiment, the plurality of mRNA polynucleotides are separately encapsulated within the lipid carrier particles and then subsequently compounded and/or mixed to produce the final bulk.

Generally, the RNA comprises a 5′ cap, such as a 7-methylguanosine, which may be added via enzymatic means or a non-enzymatic reaction. The RNA may have the following exemplary 5′ caps:
a 7-methylguanosine linked 5′-to-5′ to the 5′ first ribonucleotide by a triphosphate bridge (also referred to as “Cap O”).
a 7-methylguanosine linked 5′-to-5′ to the 5′ first ribonucleotide by a triphosphate bridge, and wherein the first 5′ ribonucleotide comprises a 2′-methylated ribose (2′-O-Me) (also referred to as “Cap 1”)
a 7-methylguanosine linked 5′-to-5′ to the 5′ first ribonucleotides by a triphosphate bridge, and wherein the first and second 5′ ribonucleotides comprise a 2′-methylated ribose (2′-O-Me) (also referred to as “Cap 2”).
or a 7-methylguanosine linked 5′-to-5′ to the 5′ first ribonucleotides by a triphosphate bridge, and wherein the first, second and third 5′ ribonucleotides comprise a 2′-methylated ribose (2′-O-Me).

In an embodiment, the 5′ cap is a 7-methylguanosine linked 5′-to-5′ to the 5′ first ribonucleoside by a triphosphate bridge, and wherein the first 5′ ribonucleoside comprises a 2′-methylated ribose (2′-O-Me), e.g., the 5′ end of the RNA has the structure m7G (5′) ppp (5′) (2′OMeA) pG. In an embodiment, this cap is added non-enzymatically through the use of the following reagent:

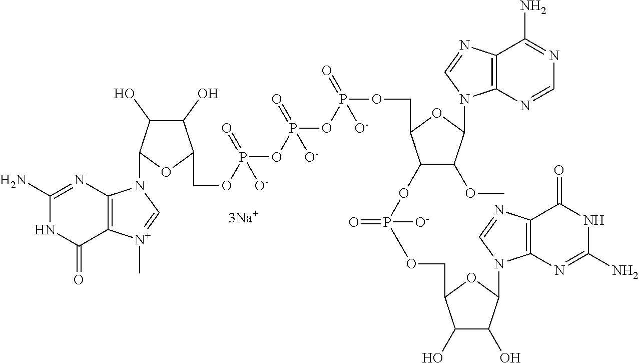 [SMILES: COC1C(OP(=O)([O-])OCC2OC(n3cnc4c(=O)[nH]c(N)nc43)C(O)C2O)C(COP(=O)([O-])OP(=O)([O-])OP(=O)([O-])OCC2OC(n3c[n+](C)c4c(=O)[nH]c(N)nc43)C(O)C2O)OC1n1cnc2c(N)ncnc21]

Said reagent is sold as CLEANCAP Reagent AG (TRILINK BIOTECHNOLOGIES).

In other embodiments, a cap may be added resulting in the 5′ end of the RNA having the structure m7(3′OMeG)(5′)ppp(5′)(2′OMeA)pG. This cap may be added non-enzymatically through the use of the following reagent:

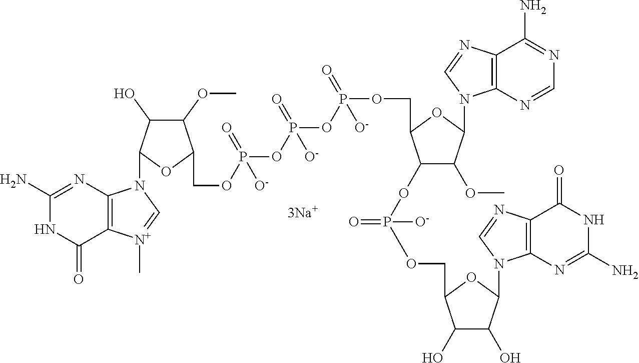 [SMILES: COC1C(COP(=O)([O-])OP(=O)([O-])OP(=O)([O-])OCC2OC(n3cnc4c(N)ncnc43)C(OC)C2OP(=O)([O-])OCC2OC(n3cnc4c(=O)[nH]c(N)nc43)C(O)C2O)OC(n2c[n+](C)c3c(=O)[nH]c(N)nc32)C1O]

Said reagent is sold as CLEANCAP Reagent AG (3′OMe) (TRILINK BIOTECHNOLOGIES)

Generally, the RNA comprises a 3′ poly-adenosine (“poly-A”) tail, e.g., comprising 10-700 A ribonucleotides. The poly-A tail may comprise at least two non-contiguous stretches of A ribonucleotides (also referred to as a “split poly-A tail”), or a (in particular, only one) contiguous stretch of A ribonucleotides. The total number of A ribonucleotides (“As”) in at least two non-contiguous stretches may be, for example, 10-700, such as 10-600, 10-500, 20-500, 50-500, 70-500, 100-500, 20-400, 30-300, 40-200, 50-150, 70-120, 100-120, or, in particular, 100-120. The total number of As in a (in particular, only one) contiguous stretch may be, for example, 10-700; such as 10-600, 20-600 or in particular 40-600 (such as 50-600, 80-600, 80-550, 100-500; or 40-70, 50-65 or 55-65). Wherein at least two non-contiguous stretches of As are used, these may be of differing length. For example, a first stretch may be 10-150 As in length, such as 10-100, 10-50, 15-50, 20-50, 20-40, 25-40, or, in particular 25-35 As in length. For example, a second stretch may be 10-150 As in length, such as 10-150, 20-120, 30-100, 40-90, 50-90, 60-90, 65-90, 70-90, or, in particular, 80-90 As in length. The first stretch may be located 5′ or 3′ relative to the second stretch. However, in a particular embodiment, the first stretch is located 5′ relative to the second stretch. In a further particular embodiment, the polyA tail comprises, in the 5′ to 3′ direction, a first and a second non-contiguous stretch of As, that are 25-35 and 80-90 As in length respectively. In a further particular embodiment, the poly A tail comprises, in the 5′-3′ direction, a first and a second non-contiguous stretch of As, that are 25-35 and 65-90 As in length respectively. In some embodiments, the at least two non-contiguous stretches of As is from, or is part of, the 3′ untranslated region (UTR), e.g., as detailed below.

In an embodiment the RNA is nucleoside modified. Therefore, RNA analogues, such as those containing modified backbones (e.g., peptide nucleic acids (PNAs) or phosphorothioates) or modified bases, are within the scope of the present disclosure. Thus, in an embodiment, the nucleic acid is RNA, optionally nucleoside modified RNA. The nucleic acid may be linear, circular and/or branched, but will generally be linear. Typically, the nucleic acid will be in recombinant form, i.e., a form which does not occur in nature.

In an embodiment, the RNA comprises (in addition to any 5′ cap structure) one or more modified ribonucleotides, i.e., ribonucleotides that are modified in structure relative to standard A, C, G or U ribonucleotides. In other embodiments, the RNA does not comprise modified ribonucleotides, i.e., the RNA contains standard A, C, G or U ribonucleotides only (except for any 5′ cap structure, if present, e.g., as detailed above).

In an embodiment, wherein one or more modified ribonucleotides are used, said one or more modified ribonucleotides may be, or may comprise, N1-methylpseudouridine (“1mΨ”); pseudouridine (“Ψ”); N1-ethylpseudouridine; 2-methylthio-N6-(cis-hydroxyisopentenyl)adenosine; 2-methylthio-N6-methyladenosine; 2-methylthio-N6-threonyl carbamoyladenosine; N6-glycinylcarbamoyladenosine; N6-isopentenyladenosine; N6-methyladenosine (m⁶A); N6-threonylcarbamoyladenosine; 1,2′-O-dimethyladenosine; 1-methyladenosine; 2′-O-methyladenosine; 2′-O-ribosyladenosine (phosphate); 2-methyladenosine; 2-methylthio-N6 isopentenyladenosine; 2-methylthio-N6-hydroxynorvalyl carbamoyladenosine; 2′-O-methyladenosine; 2′-O-ribosyladenosine (phosphate); Isopentenyladenosine; N6-(cis-hydroxyisopentenyl)adenosine; N6,2′-O-dimethyladenosine; N6,2′-O-dimethyladenosine; N6,N6,2′-O-trimethyladenosine; N6,N6-dimethyladenosine; N6-acetyladenosine; N6-hydroxynorvalylcarbamoyladenosine; N6-methyl-N6-threonylcarbamoyladenosine; 2-methyladenosine; 2-methylthio-N6-isopentenyladenosine; 7-deaza-adenosine; N1-methyl-adenosine; N6,N6 (dimethyl)adenine; N6-cis-hydroxy-isopentenyl-adenosine; .alpha.-thio-adenosine; 2 (amino)adenine; 2 (aminopropyl)adenine; 2 (methylthio) N6 (isopentenyl)adenine; 2-(alkyl)adenine; 2-(aminoalkyl)adenine; 2-(aminopropyl)adenine; 2-(halo)adenine; 2-(halo)adenine; 2-(propyl)adenine; 2′-Amino-2′-deoxy-ATP; 2′-Azido-2′-deoxy-ATP; 2′-Deoxy-2′-a-aminoadenosine TP; 2′-Deoxy-2′-a-azidoadenosine TP; 6 (alkyl)adenine; 6 (methyl)adenine; 6-(alkyl)adenine; 6-(methyl)adenine; 7 (deaza)adenine; 8 (alkenyl)adenine; 8 (alkynyl)adenine; 8 (amino)adenine; 8 (thioalkyl)adenine; 8-(alkenyl)adenine; 8-(alkyl)adenine; 8-(alkynyl)adenine; 8-(amino)adenine; 8-(halo)adenine; 8-(hydroxyl)adenine; 8-(thioalkyl)adenine; 8-(thiol)adenine; 8-azido-adenosine; aza adenine; deaza adenine; N6 (methyl)adenine; N6-(isopentyl)adenine; 7-deaza-8-aza-adenosine; 7-methyladenine; 1-Deazaadenosine TP; 2′Fluoro-N6-Bz-deoxyadenosine TP; 2′-OMe-2-Amino-ATP; 2′O-methyl-N6-Bz-deoxyadenosine TP; 2′-a-Ethynyladenosine TP; 2-aminoadenine; 2-Aminoadenosine TP; 2-Amino-ATP; 2′-a-Trifluoromethyladenosine TP; 2-Azidoadenosine TP; 2′-b-Ethynyladenosine TP; 2-Bromoadenosine TP; 2′-b-Trifluoromethyladenosine TP; 2-Chloroadenosine TP; 2′-Deoxy-2′,2′-difluoroadenosine TP; 2′-Deoxy-2′-a-mercaptoadenosine TP; 2′-Deoxy-2′-a-thiomethoxyadenosine TP; 2′-Deoxy-2′-b-aminoadenosine TP; 2′-Deoxy-2′-b-azidoadenosine TP; 2′-Deoxy-2′-b-bromoadenosine TP; 2′-Deoxy-2′-b-chloroadenosine TP; 2′-Deoxy-2′-b-fluoroadenosine TP; 2′-Deoxy-2′-b-iodoadenosine TP; 2′-Deoxy-2′-b-mercaptoadenosine TP; 2′-Deoxy-2′-b-thiomethoxyadenosine TP; 2-Fluoroadenosine TP; 2-Iodoadenosine TP; 2-Mercaptoadenosine TP; 2-methoxy-adenine; 2-methylthio-adenine; 2-Trifluoromethyladenosine TP; 3-Deaza-3-bromoadenosine TP; 3-Deaza-3-chloroadenosine TP; 3-Deaza-3-fluoroadenosine TP; 3-Deaza-3-iodoadenosine TP; 3-Deazaadenosine TP; 4′-Azidoadenosine TP; 4′-Carbocyclic adenosine TP; 4′-Ethynyladenosine TP; 5′-Homo-adenosine TP; 8-Aza-ATP; 8-bromo-adenosine TP; 8-Trifluoromethyladenosine TP; 9-Deazaadenosine TP; 2-aminopurine; 7-deaza-2,6-diaminopurine; 7-deaza-8-aza-2,6-diaminopurine; 7-deaza-8-aza-2-aminopurine; 2,6-diaminopurine; 7-deaza-8-aza-adenine, 7-deaza-2-aminopurine; 2-thiocytidine; 3-methylcytidine; 5-formylcytidine; 5-hydroxymethylcytidine; 5-methylcytidine; N4-acetylcytidine; 2′-O-methylcytidine; 2′-O-methylcytidine; 5,2′-O-dimethylcytidine; 5-formyl-2′-O-methylcytidine; Lysidine; N4,2′-O-dimethylcytidine; N4-acetyl-2′-O-methylcytidine; N4-methylcytidine; N4,N4-Dimethyl-2′-OMe-Cytidine TP; 4-methylcytidine; 5-aza-cytidine; Pseudo-iso-cytidine; pyrrolo-cytidine; .alpha.-thio-cytidine; 2-(thio)cytosine; 2′-Amino-2′-deoxy-CTP; 2′-Azido-2′-deoxy-CTP; 2′-Deoxy-2′-a-aminocytidine TP; 2′-Deoxy-2′-a-azidocytidine TP; 3 (deaza) 5 (aza)cytosine; 3 (methyl)cytosine; 3-(alkyl)cytosine; 3-(deaza) 5 (aza)cytosine; 3-(methyl)cytidine; 4,2′-O-dimethylcytidine; 5 (halo)cytosine; 5 (methyl)cytosine; 5 (propynyl)cytosine; 5 (trifluoromethyl)cytosine; 5-(alkyl)cytosine; 5-(alkynyl)cytosine; 5-(halo)cytosine; 5-(propynyl)cytosine; 5-(trifluoromethyl)cytosine: 5-bromo-cytidine; 5-iodo-cytidine; 5-propynyl cytosine; 6-(azo)cytosine; 6-aza-cytidine; aza cytosine; deaza cytosine; N4 (acetyl)cytosine; 1-methyl-1-deaza-pseudoisocytidine; 1-methyl-pseudoisocytidine; 2-methoxy-5-methyl-cytidine: 2-methoxy-cytidine; 2-thio-5-methyl-cytidine; 4-methoxy-1-methyl-pseudoisocytidine; 4-methoxy-pseudoisocytidine; 4-thio-1-methyl-1-deaza-pseudoisocytidine; 4-thio-1-methyl-pseudoisocytidine; 4-thio-pseudoisocytidine; 5-aza-zebularine; 5-methyl-zebularine; pyrrolo-pseudoisocytidine; Zebularine; (E)-5-(2-Bromo-vinyl)cytidine TP; 2,2′-anhydro-cytidine TP hydrochloride; 2′Fluor-N4-Bz-cytidine TP; 2′Fluoro-N4-Acetyl-cytidine TP; 2′-O-Methyl-N4-Acetyl-cytidine TP; 2′O-methyl-N4-Bz-cytidine TP; 2′-a-Ethynylcytidine TP; 2′-a-Trifluoromethylcytidine TP; 2′-b-Ethynylcytidine TP; 2′-b-Trifluoromethylcytidine TP; 2′-Deoxy-2′,2′-difluorocytidine TP; 2′-Deoxy-2′-a-mercaptocytidine TP; 2′-Deoxy-2′-a-thiomethoxycytidine TP; 2′-Deoxy-2′-b-aminocytidine TP; 2′-Deoxy-2′-b-azidocytidine TP; 2′-Deoxy-2′-b-bromocytidine TP; 2′-Deoxy-2′-b-chlorocytidine TP; 2′-Deoxy-2′-b-fluorocytidine TP; 2′-Deoxy-2′-b-iodocytidine TP; 2′-Deoxy-2′-b-mercaptocytidine TP; 2′-Deoxy-2′-b-thiomethoxycytidine TP; 2′-O-Methyl-5-(1-propynyl)cytidine TP; 3′-Ethynylcytidine TP; 4′-Azidocytidine TP; 4′-Carbocyclic cytidine TP; 4′-Ethynylcytidine TP; 5-(1-Propynyl)ara-cytidine TP; 5-(2-Chloro-phenyl)-2-thiocytidine TP; 5-(4-Amino-phenyl)-2-thiocytidine TP; 5-Aminoallyl-CTP; 5-Cyanocytidine TP; 5-Ethynylara-cytidine TP; 5-Ethynylcytidine TP; 5′-Homo-cytidine TP; 5-Methoxycytidine TP; 5-Trifluoromethyl-Cytidine TP; N4-Amino-cytidine TP; N4-Benzoyl-cytidine TP; Pseudoisocytidine; 7-methylguanosine; N2,2′-O-dimethylguanosine; N2-methylguanosine; Wyosine; 1,2′-O-dimethylguanosine; 1-methylguanosine; 2′-O-methylguanosine; 2′-O-ribosylguanosine (phosphate); 2′-O-methylguanosine; 2′-O-ribosylguanosine (phosphate); 7-aminomethyl-7-deazaguanosine; 7-cyano-7-deazaguanosine; Archaeosine; Methylwyosine; N2,7-dimethylguanosine; N2,N2,2′-O-trimethylguanosine; N2,N2,7-trimethylguanosine; N2,N2-dimethylguanosine; N2,7,2′-O-trimethylguanosine; 6-thio-guanosine; 7-deaza-guanosine; 8-oxo-guanosine; N1-methyl-guanosine; .alpha.-thio-guanosine; 2 (propyl)guanine; 2-(alkyl)guanine; 2′-Amino-2′-deoxy-GTP; 2′-Azido-2′-deoxy-GTP; 2′-Deoxy-2′-a-aminoguanosine TP; 2′-Deoxy-2′-a-azidoguanosine TP; 6 (methyl)guanine; 6-(alkyl)guanine; 6-(methyl)guanine; 6-methyl-guanosine; 7 (alkyl)guanine; 7 (deaza)guanine; 7 (methyl)guanine; 7-(alkyl)guanine; 7-(deaza)guanine; 7-(methyl)guanine; 8 (alkyl)guanine; 8 (alkynyl)guanine; 8 (halo)guanine; 8 (thioalkyl)guanine; 8-(alkenyl)guanine; 8-(alkyl)guanine; 8-(alkynyl)guanine; 8-(amino)guanine; 8-(halo)guanine; 8-(hydroxyl)guanine; 8-(thioalkyl)guanine; 8-(thiol)guanine; aza guanine; deaza guanine; N (methyl)guanine; N-(methyl)guanine; 1-methyl-6-thio-guanosine; 6-methoxy-guanosine; 6-thio-7-deaza-8-aza-guanosine; 6-thio-7-deaza-guanosine; 6-thio-7-methyl-guanosine; 7-deaza-8-aza-guanosine; 7-methyl-8-oxo-guanosine; N2,N2-dimethyl-6-thio-guanosine; N2-methyl-6-thio-guanosine; 1-Me-GTP; 2′Fluoro-N2-isobutyl-guanosine TP; 2′O-methyl-N2-isobutyl-guanosine TP; 2′-a-Ethynylguanosine TP; 2′-a-Trifluoromethylguanosine TP; 2′-b-Ethynylguanosine TP; 2′-b-Trifluoromethylguanosine TP; 2′-Deoxy-2′,2′-difluoroguanosine TP; 2′-Deoxy-2′-a-mercaptoguanosine TP; 2′-Deoxy-2′-a-thiomethoxyguanosine TP; 2′-Deoxy-2′-b-aminoguanosine TP; 2′-Deoxy-2′-b-azidoguanosine TP; 2′-Deoxy-2′-b-bromoguanosine TP; 2′-Deoxy-2′-b-chloroguanosine TP; 2′-Deoxy-2′-b-fluoroguanosine TP; 2′-Deoxy-2′-b-iodoguanosine TP; 2′-Deoxy-2′-b-mercaptoguanosine TP; 2′-Deoxy-2′-b-thiomethoxyguanosine TP; 4′-Azidoguanosine TP; 4′-Carbocyclic guanosine TP; 4′-Ethynylguanosine TP; 5′-Homo-guanosine TP; 8-bromo-guanosine TP; 9-Deazaguanosine TP; N2-isobutyl-guanosine TP; 1-methylinosine; Inosine; 1,2′-O-dimethylinosine; 2′-O-methylinosine; 7-methylinosine; 2′-O-methylinosine; Epoxyqueuosine; galactosyl-queuosine; Mannosylqueuosine; Queuosine; allyamino-thymidine; aza thymidine; deaza thymidine; deoxy-thymidine; 2′-O-methyluridine; 2-thiouridine; 3-methyluridine; 5-carboxymethyluridine; 5-hydroxyuridine; 5-methyluridine; 5-taurinomethyl-2-thiouridine; 5-taurinomethyluridine; Dihydrouridine; (3-(3-amino-3-carboxypropyl)uridine; 1-methyl-3-(3-amino-5-carboxypropyl)pseudouridine; 1-methylpseduouridine; 1-methyl-pseudouridine; 2′-O-methyluridine; 2′-O-methylpseudouridine; 2′-O-methyluridine; 2-thio-2′-O-methyluridine; 3-(3-amino-3-carboxypropyl)uridine; 3,2′-O-dimethyluridine; 3-Methyl-pseudo-Uridine TP; 4-thiouridine; 5-(carboxyhydroxymethyl)uridine; 5-(carboxyhydroxymethyl)uridine methyl ester, 5,2′-O-dimethyluridine; 5,6-dihydro-uridine; 5-aminomethyl-2-thiouridine; 5-carbamoylmethyl-2′-O-methyluridine; 5-carbamoylmethyluridine; 5-carboxyhydroxymethyluridine; 5-carboxyhydroxymethyluridine methyl ester, 5-carboxymethylaminomethyl-2′-O-methyluridine; 5-carboxymethylaminomethyl-2-thiouridine; 5-carboxymethylaminomethyl-2-thiouridine; 5-caboxymethylaminomethyluridine; 5-carboxymethylaminomethyluridine; 5-Carbamoylmethyluridine TP; 5-methoxycaeoonylmethyl-2′-O-methyluridine; 5-methoxycarbonylmethyl-2-thiouridine; 5-methoxycarbonylmethyluridine; 5-methoxyuridine; 5-methyl-2-thiouridine; 5-methylaminomethyl-2-selenouridine; 5-methylaminomethyl-2-thiouridine; 5-methylaminomethyluridine; 5-Methyldihydrouridine; 5-Oxyacetic acid-Uridine TP; 5-Oxyacetic acid-methyl ester-Uridine TP; N1-methyl-pseudo-uridine; N1-ethyl-pseudo-uridine; uridine 5-oxyacetic acid; uridine 5-oxyacetic acid methyl ester; 3-(3-Amino-3-carboxypropyl)-Uridine TP; 5-(iso-Pentenylaminomethyl)-2-thiouridine TP; 5-(iso-Pentenylaminomethyl)-2′-O-methyluridine TP; 5-(iso-Pentenylaminomethyl)uridine TP; 5-propynyl uracil; .alpha.-thio-uridine; 1 (aminoalkylamino-carbonylethylenyl)-2(thio)-pseudouridine; 1 (aminoalkylaminocarbonylethylenyl)-2,4-(dithio)pseudouridine; 1 (aminoalkylaminocarbonylethylenyl)-4 (thio)pseudouridine; 1 (aminoalkylaminocarbonylethylenyl)-pseudouridine; 1 (aminocazbonylethylenyl)-2(thio)-pseudouridine; 1 (aminocarbonylethylenyl)-2,4-(dithio)pseudouridine; 1 (aminocarbonylethylenyl)-4 (thio)pseudouridine; 1 (aminocarbonylethylenyl)-pseudouridine; 1 substituted 2(thio)-pseudouridine; 1 substituted 2,4-(dithio)pseudouridine; 1 substituted 4 (thio)pseudouridine; 1 substituted pseudouridine; 1-(aminoalkylamino-carbonylethylenyl)-2-(thio)-pseudouridine; 1-Methyl-3-(3-amino-3-carboxypropyl) pseudouridine TP; 1-Methyl-3-(3-amino-3-carboxypropyl)pseudo-UTP; 1-Methyl-pseudo-UTP; 2 (thio)pseudouridine; 2′ deoxy uridine; 2′ fluorouridine; 2-(thio)uracil; 2,4-(dithio)psuedouracil; 2′ methyl, 2′amino, 2′azido, 2′fluoro-guanosine; 2′-Amino-2′-deoxy-UTP; 2′-Azido-2′-deoxy-UTP; 2′-Azido-deoxyuridine TP; 2′-O-methylpseudouridine; 2′ deoxy uridine; 2′ fluorouridine; 2′-Deoxy-2′-a-aminouridine TP; 2′-Deoxy-2′-a-azidouridine TP; 2-methylpseudouridine; 3 (3 amino-3 carboxypropyl)uracil; 4 (thio)pseudouridine; 4-(thio)pseudouridine; 4-(thio)uracil; 4-thiouracil; 5 (1,3-diazole-1-alkyl)uracil; 5 (2-aminopropyl)uracil; 5 (aminoalkyl)uracil; 5 (dimethylaminoalkyl)uracil; 5 (guanidiniumalkyl)uracil; 5 (methoxycarbonylmethyl)-2-(thio)uracil; 5 (methoxycarbonyl-methyl)uracil; 5 (methyl) 2 (thio)uracil; 5 (methyl) 2,4 (dithio)uracil; 5 (methyl) 4 (thio)uracil; 5 (methylaminomethyl)-2 (thio)uracil; 5 (methylaminomethyl)-2,4 (dithio)uracil; 5 (methylaminomethyl)-4 (thio)uracil; 5 (propynyl)uracil; 5 (trifluoromethyl)uracil; 5-(2-aminopropyl)uracil; 5-(alkyl)-2-(thio)pseudouridine; 5-(alkyl)-2,4 (dithio)pseudouridine; 5-(alkyl)-4 (thio)pseudouridine; 5-(alkyl)pseudouridine; 5-(alkyl)uracil; 5-(alkynyl)uracil; 5-(allylamino)uracil; 5-(cyanoalkyl)uracil; 5-(dialkylaminoalkyl)uracil; 5-(dimethylaminoalkyl)uracil; 5-(guanidiniumalkyl)uracil; 5-(halo)uracil; 5-(1,3-diazole-1-alkyl)uracil; 5-(methoxy)uracil; 5-(methoxycarbonylmethyl)-2-(thio)uracil; 5-(methoxycarbonyl-methyl)uracil; 5-(methyl) 2(thio)uracil; 5-(methyl) 2,4 (dithio)uracil; 5-(methyl) 4 (thio)uracil; 5-(methyl)-2-(thio)pseudouridine; 5-(methyl)-2,4 (dithio)pseudouridine; 5-(methyl)-4 (thio)pseudouridine; 5-(methyl)pseudouridine; 5-(methylaminomethyl)-2 (thio)uracil; 5-(methylaminomethyl)-2,4(dithio)uracil; 5-(methylaminomethyl)-4-(thio)uracil; 5-(propynyl)uracil; 5-(trifluoromethyl)uracil; 5-aminoallyl-uridine; 5-bromo-uridine; 5-iodo-uridine; 5-uracil; 6 (azo)uracil; 6-(azo)uracil; 6-aza-uridine; allyamino-uracil; aza uracil; deaza uracil; N3 (methyl)uracil; Pseudo-UTP-1-2-ethanoic acid; Pseudouridine; 4-Thio-pseudo-UTP; 1-carboxymethyl-pseudouridine; 1-methyl-1-deaza-pseudouridine; 1-propynyl-uridine; 1-taurinomethyl-1-methyl-uridine; 1-taurinomethyl-4-thio-uridine; 1-taurinomethyl-pseudouridine; 2-methoxy-4-thio-pseudouridine; 2-thio-1-methyl-1-deaza-pseudouridine; 2-thio-1-methyl-pseudouridine; 2-thio-5-aza-uridine; 2-thio-dihydropseudouridine; 2-thio-dihydrouridine; 2-thio-pseudouridine; 4-methoxy-2-thio-pseudouridine; 4-methoxy-pseudouridine; 4-thio-1-methyl-pseudouridine; 4-thio-pseudouridine; 5-aza-uridine; Dihydropseudouridine; (.+-.)1-(2-Hydroxypropyl)pseudouridine TP; (2R)-1-(2-Hydroxypropyl)pseudouridine TP; (2S)-1-(2-Hydroxypropyl)pseudouridine TP; (E)-5-(2-Bromo-vinyl)ara-uridine TP; (E)-5-(2-Bromo-vinyl)uridine TP; (Z)-5-(2-Bromo-vinyl)ara-uridine TP; (Z)-5-(2-Bromo-vinyl)uridine TP; 1-(2,2,2-Trifluoroethyl)-pseudo-UTP; 1-(2,2,3,3,3-Pentafluoropropyl)pseudouridine TP; 1-(2,2-Diethoxyethyl)pseudouridine TP; 1-(2,4,6-Trimethylbenzyl)pseudouridine TP; 1-(2,4,6-Trimethyl-benzyl)pseudo-UTP; 1-(2,4,6-Trimethyl-phenyl)pseudo-UTP; 1-(2-Amino-2-carboxyethyl)pseudo-UTP; 1-(2-Amino-ethyl)pseudo-UTP; 1-(2-Hydroxyethyl)pseudouridine TP; 1-(2-Methoxyethyl)pseudouridine TP; 1-(3,4-Bis-trifluoromethoxybenzyl)pseudouridine TP; 1-(3,4-Dimethoxybenzyl)pseudouridine TP; 1-(3-Amino-3-carboxypropyl)pseudo-UTP; 1-(3-Amino-propyl)pseudo-UTP; 1-(3-Cyclopropyl-prop-2-ynyl)pseudouridine TP; 1-(4-Amino-4-carboxybutyl)pseudo-UTP; 1-(4-Amino-benzyl)pseudo-UTP; 1-(4-Amino-butyl)pseudo-UTP; 1-(4-Amino-phenyl)pseudo-UTP; 1-(4-Azidobenzyl)pseudouridine TP; 1-(4-Bromobenzyl)pseudouridine TP; 1-(4-Chlorobenzyl)pseudouridine TP; 1-(4-Fluorobenzyl)pseudouridine TP; 1-(4-Iodobenzyl)pseudouridine TP; 1-(4-Methanesulfonylbenzyl)pseudouridine TP; 1-(4-Methoxybenzyl)pseudouridine TP; 1-(4-Methoxy-benzyl)pseudo-UTP; 1-(4-Methoxy-phenyl)pseudo-UTP; 1-(4-Methylbenzyl)pseudouridine TP; 1-(4-Methyl-benzyl)pseudo-UTP; 1-(4-Nitrobenzyl)pseudouridine TP; 1-(4-Nitro-benzyl)pseudo-UTP; 1-(4-Nitro-phenyl)pseudo-UTP; 1-(4-Thiomethoxybenzyl)pseudouridine TP; 1-(4-Trifluoromethoxybenzyl)pseudouridine TP; 1-(4-Trifluoromethylbenzyl)pseudouridine TP; 1-(5-Amino-pentyl)pseudo-UTP; 1-(6-Amino-hexyl)pseudo-UTP; 1,6-Dimethyl-pseudo-UTP; 1-[3-(2-{2-[2-(2-Aminoethoxy)-ethoxy]-ethoxy}-ethoxy)-propionyl]pseudouri-dine TP; 1-{3-[2-(2-Aminoethoxy)-ethoxy]-propionyl}pseudouridine TP; 1-Acetylpseudouridine TP; I-Alkyl-6-(1-propynyl)-pseudo-UTP; 1-Alkyl-6-(2-propynyl)-pseudo-UTP; 1-Alkyl-6-allyl-pseudo-UTP; 1-Alkyl-6-ethynyl-pseudo-UTP; 1-Alkyl-6-homoallyl-pseudo-UTP; 1-Alkyl-6-vinyl-pseudo-UTP; 1-Allylpseudouridine TP; 1-Aminomethyl-pseudo-UTP; 1-Benzoylpseudouridine TP; 1-Benzyloxymethylpseudouridine TP; 1-Benzyl-pseudo-UTP; 1-Biotinyl-PEG2-pseudouridine TP; 1-Biotinylpseudouridine TP; 1-Butyl-pseudo-UTP; 1-Cyanomethylpseudouridine TP; 1-Cyclobutylmethyl-pseudo-UTP; 1-Cyclobutyl-pseudo-UTP; 1-Cycloheptylmethyl-pseudo-UTP; 1-Cycloheptyl-pseudo-UTP; 1-Cyclohexylmethyl-pseudo-UTP; 1-Cyclohexyl-pseudo-UTP; 1-Cyclooctylmethyl-pseudo-UTP; 1-Cyclooctyl-pseudo-UTP; 1-Cyclopentylmethyl-pseudo-UTP; 1-Cyclopentyl-pseudo-UTP; 1-Cyclopropylmethyl-pseudo-UTP; 1-Cyclopropyl-pseudo-UTP; 1-Ethyl-pseudo-UTP; 1-Hexyl-pseudo-UTP; 1-Homoallylpseudouridine TP; 1-Hydroxymethylpseudouridine TP; 1-iso-propyl-pseudo-UTP; 1-Me-2-thio-pseudo-UTP; 1-Me-4-thio-pseudo-UTP; 1-Me-alpha-thio-pseudo-UTP; 1-Methanesulfonylmethylpseudouridine TP; 1-Methoxymethylpseudouridine TP; 1-Methyl-6-(2,2,2-Trifluoroethyl)pseudo-UTP; 1-Methyl-6-(4-morpholino)-pseudo-UTP; 1-Methyl-6-(4-thiomorpholino)-pseudo-UTP; 1-Methyl-6-(substituted phenyl)pseudo-UTP; 1-Methyl-6-amino-pseudo-UTP; 1-Methyl-6-azido-pseudo-UTP; 1-Methyl-6-bromo-pseudo-UTP; 1-Methyl-6-butyl-pseudo-UTP; 1-Methyl-6-chloro-pseudo-UTP; 1-Methyl-6-cyano-pseudo-UTP; 1-Methyl-6-dimethylamino-pseudo-UTP; 1-Methyl-6-ethoxy-pseudo-UTP; 1-Methyl-6-ethylcarboxylate-pseudo-UTP; 1-Methyl-6-ethyl-pseudo-UTP; 1-Methyl-6-fluoro-pseudo-UTP; 1-Methyl-6-formyl-pseudo-UTP; 1-Methyl-6-hydroxyamino-pseudo-UTP; 1-Methyl-6-hydroxy-pseudo-UTP; 1-Methyl-6-iodo-pseudo-UTP; 1-Methyl-6-iso-propyl-pseudo-UTP; 1-Methyl-6-methoxy-pseudo-UTP; 1-Methyl-6-methylamino-pseudo-UTP; 1-Methyl-6-phenyl-pseudo-UTP; 1-Methyl-6-propyl-pseudo-UTP; 1-Methyl-6-tert-butyl-pseudo-UTP; 1-Methyl-6-trifluoromethoxy-pseudo-UTP; 1-Methyl-6-trifluoromethyl-pseudo-UTP; 1-Morpholinomethylpseudouridine TP; 1-Pentyl-pseudo-UTP; 1-Phenyl-pseudo-UTP; 1-Pivaloylpseudouridine TP; 1-Propargylpseudouridine TP; 1-Propyl-pseudo-UTP; 1-propynyl-pseudouridine; 1-p-tolyl-pseudo-UTP; 1-tert-Butyl-pseudo-UTP; 1-Thiomethoxymethylpseudouridine TP; 1-Thiomorpholinomethylpseudouridine TP; 1-Trifluoroacetylpseudouridine TP; 1-Trifluoromethyl-pseudo-UTP; 1-Vinylpseudouridine TP; 2,2′-anhydro-uridine TP; 2′-bromo-deoxyuridine TP; 2′-F-5-Methyl-2′-deoxy-UTP; 2′-OMe-5-Me-UTP; 2′-OMe-pseudo-UTP; 2′-a-Ethynyluridine TP; 2′-a-Trifluoromethyluridine TP; 2′-b-Ethynyluridine TP; 2′-b-Trifluoromethyluridine TP; 2′-Deoxy-2′,2′-difluorouridine TP; 2′-Deoxy-2′-a-mercaptouridine TP; 2′-Deoxy-2′-a-thiomethoxyuridine TP; 2′-Deoxy-2′-b-aminouridine TP; 2′-Deoxy-2′-b-azidouridine TP; 2′-Deoxy-2′-b-bromouridine TP; 2′-Deoxy-2′-b-chlorouridine TP; 2′-Deoxy-2′-b-fluorouridine TP; 2′-Deoxy-2′-b-iodouridine TP; 2′-Deoxy-2′-b-mercaptouridine TP; 2′-Deoxy-2′-b-thiomethoxyuridine TP; 2-methoxy-4-thio-uridine; 2-methoxyuridine; 2′-O-Methyl-5-(1-propynyl)uridine TP; 3-Alkyl-pseudo-UTP; 4′-Azidouridine TP; 4′-Carbocyclic uridine TP; 4′-Ethynyluridine TP; 5-(1-Propynyl)ara-uridine TP; 5-(2-Furanyl)uridine TP; 5-Cyanouridine TP; 5-Dimethylaminouridine TP; 5′-Homo-uridine TP; 5-iodo-2′-fluoro-deoxyuridine TP; 5-Phenylethynyluridine TP; 5-Trideuteromethyl-6-deuterouridine TP; 5-Trifluoromethyl-Uridine TP; 5-Vinylarauridine TP; 6-(2,2,2-Trifluoroethyl)-pseudo-UTP; 6-(4-Morpholino)-pseudo-UTP; 6-(4-Thiomorpholino)-pseudo-UTP; 6-(Substituted-Phenyl)-pseudo-UTP; 6-Amino-pseudo-UTP; 6-Azido-pseudo-UTP; 6-Bromo-pseudo-UTP; 6-Butyl-pseudo-UTP; 6-Chloro-pseudo-UTP; 6-Cyano-pseudo-UTP; 6-Dimethylamino-pseudo-UTP; 6-Ethoxy-pseudo-UTP; 6-Ethylcarboxylate-pseudo-UTP; 6-Ethyl-pseudo-UTP; 6-Fluoro-pseudo-UTP; 6-Formyl-pseudo-UTP; 6-Hydroxyamino-pseudo-UTP; 6-Hydroxy-pseudo-UTP; 6-Iodo-pseudo-UTP; 6-iso-Propyl-pseudo-UTP; 6-Methoxy-pseudo-UTP; 6-Methylamino-pseudo-UTP; 6-Methyl-pseudo-UTP; 6-Phenyl-pseudo-UTP; 6-Phenyl-pseudo-UTP; 6-Propyl-pseudo-UTP; 6-tert-Butyl-pseudo-UTP; 6-Trifluoromethoxy-pseudo-UTP; 6-Trifluoromethyl-pseudo-UTP; Alpha-thio-pseudo-UTP; Pseudouridine 1-(4-methylbenzenesulfonic acid) TP; Pseudouridine 1-(4-methylbenzoic acid) TP; Pseudouridine TP 1-[3-(2-ethoxy)]propionic acid; Pseudouridine TP 1-[3-{2-(2-[2-(2-ethoxy)-ethoxy]-ethoxy)-ethoxy}]propionic acid; Pseudouridine TP 1-[3-{2-(2-[2-{2-(2-ethoxy)-ethoxy)-ethoxy}-ethoxy]-ethoxy}]propionic acid; Pseudouridine TP 1-[3-{2-(2-[2-ethoxy]-ethoxy)-ethoxy}]propionic acid; Pseudouridine TP 1-[3-{2-(2-ethoxy)-ethoxy}]propionic acid; Pseudouridine TP 1-methylphosphonic acid; Pseudouridine TP 1-methylphosphonic acid diethyl ester; Pseudo-UTP-N1-3-propionic acid; Pseudo-UTP-N1-4-butanoic acid; Pseudo-UTP-N1-5-pentanoic acid; Pseudo-UTP-N1-6-hexanoic acid; Pseudo-UTP-N1-7-heptanoic acid; Pseudo-UTP-N1-methyl-p-benzoic acid; Pseudo-UTP-N1-p-benzoic acid; Wybutosine; Hydroxywybutosine; Isowyosine; Peroxywybutosine; undermodified hydroxywybutosine; 4-demethylwyosine; 2,6-(diamino)purine; 1-(aza)-2-(thio)-3-(aza)-phenoxazin-1-yl: 1,3-(diaza)-2-(oxo)-phenthiazin-1-yl; 1,3-(diaza)-2-(oxo)-phenoxazin-1-yl; 1,3,5-(triaza)-2,6-(dioxa)-naphthalene; 2 (amino)purine; 2,4,5-(trimethyl)phenyl; 2′ methyl, 2′amino, 2′azido, 2′fluoro-cytidine; 2′ methyl, 2′amino, 2′azido, 2′fluoro-adenine; 2′methyl, 2′amino, 2′azido, 2′fluoro-uridine; 2′-amino-2′-deoxyribose; 2-amino-6-Chloro-purine; 2-aza-inosinyl; 2′-azido-2′-deoxyribose; 2′fluoro-2′-deoxyribose; 2′-fluoro-modified bases; 2′-O-methyl-ribose; 2-oxo-7-aminopyridopyrimidin-3-yl; 2-oxo-pyridopyrimidine-3-yl; 2-pyridinone; 3 nitropyrrole; 3-(methyl)-7-(propynyl)isocarbostyrilyl; 3-(methyl)isocarbostyrilyl; 4-(fluoro)-6-(methyl)benzimidazole; 4-(methyl)benzimidazole; 4-(methyl)indolyl; 4,6-(dimethyl)indolyl; 5 nitroindole; 5 substituted pyrimidines; 5-(methyl)isocarbostyrilyl; 5-nitroindole; 6-(aza)pyrimidine; 6-(azo)thymine; 6-(methyl)-7-(aza)indolyl; 6-chloro-purine; 6-phenyl-pyrrolo-pyrimidin-2-on-3-yl; 7-(aminoalkylhydroxy)-1-(aza)-2-(thio)-3-(aza)-phenthiazin-1-yl; 7-(aminoalkylhydroxy)-1-(aza)-2-(thio)-3-(aza)-phenoxazin-1-yl; 7-(aminoalkylhydroxy)-1,3-(diaza)-2-(oxo)-phenoxazin-1-yl; 7-(aminoalkylhydroxy)-1,3-(diaza)-2-(oxo)-phenthiazin-1-yl; 7-(aminoalkylhydroxy)-1,3-(diaza)-2-(oxo)-phenoxazin-1-yl; 7-(aza)indolyl; 7-(guanidiniumalkylhydroxy)-1-(aza)-2-(thio)-3-(aza)-phenoxazinl-yl; 7-(guanidiniumalkylhydroxy)-1-(aza)-2-(thio)-3-(aza)-phenthiazin-1-yl; 7-(guanidiniumalkylhydroxy)-1-(aza)-2-(thio)-3-(aza)-phenoxazin-1-yl; 7-(guanidiniumalkylhydroxy)-1,3-(diaza)-2-(oxo)-phenoxazin-1-yl; 7-(guanidiniumalkyl-hydroxy)-1,3-(diaza)-2-(oxo)-phenthiazin-1-yl; 7-(guanidiniumalkylhydroxy)-1,3-(diaza)-2-(oxo)-phenoxazin-1-yl; 7-(propynyl)isocarbostyrilyl; 7-(propynyl)isocarbostyrilyl, propynyl-7-(aza)indolyl; 7-deaza-inosinyl; 7-substituted 1-(aza)-2-(thio)-3-(aza)-phenoxazin-1-yl; 7-substituted 1,3-(diaza)-2-(oxo)-phenoxazin-1-yl; 9-(methyl)-imidizopyridinyl; Aminoindolyl; Anthracenyl; bis-ortho-(aminoalkylhydroxy)-6-phenyl-pyrrolo-pyrimidin-2-on-3-yl; bis-ortho-substituted-6-phenyl-pyrrolo-pyrimidin-2-on-3-yl; Difluorotolyl; Hypoxanthine; Imidizopyridinyl; Inosinyl; Isocarbostyrilyl; Isoguanisine; N2-substituted purines; N6-methyl-2-amino-purine; N6-substituted purines; N-alkylated derivative; Napthalenyl; Nitrobenzimidazolyl; Nitroimidazolyl; Nitroindazolyl; Nitropyrazolyl; Nubularine; 06-substituted purines; O-alkylated derivative; ortho-(aminoalkylhydroxy)-6-phenyl-pyrrolo-pyrimidin-2-on-3-yl; ortho-substituted-6-phenyl-pyrrolo-pyrimidin-2-on-3-yl; Oxoformycin TP; para-(aminoalkylhydroxy)-6-phenyl-pyrrolo-pyrimidin-2-on-3-yl; para-substituted-6-phenyl-pyrrolo-pyrimidin-2-on-3-yl; Pentacenyl; Phenanthracenyl; Phenyl; propynyl-7-(aza)indolyl; Pyrenyl; pyridopyrimidin-3-yl; pyridopyrimidin-3-yl, 2-oxo-7-amino-pyridopyrimidin-3-yl; pyrrolo-pyrimidin-2-on-3-yl; Pyrrolopyrimidinyl; Pyrrolopyrizinyl; Stilbenzyl; substituted 1,2,4-triazoles; Tetracenyl; Tubercidine; Xanthine; Xanthosine-5′-TP; 2-thio-zebularine; 5-aza-2-thio-zebularine; 7-deaza-2-amino-purine; pyridin-4-one ribonucleoside; 2-Amino-riboside-TP; Formycin A TP; Formycin B TP; Pyrrolosine TP; 2′-OH-ara-adenosine TP; 2′-OH-ara-cytidine TP; 2′-OH-ara-uridine TP; 2′-OH-ara-guanosine TP; 5-(2-carbomethoxyvinyl)uridine TP; or N6-(19-Amino-pentaoxanonadecyl)adenosine TP.

In an embodiment, the one or more modified ribonucleotides detailed above is, or comprise, 1 mΨ and/or P. In a preferred embodiment, wherein one or more modified ribonucleotides are used, said one or more modified ribonucleotides comprises or consists of 1 mΨ. In such embodiments, the RNA may comprise 1 mΨ and/or Ψ, and neither standard U ribonucleotides nor other modified U ribonucleotides (i.e. there are no standard U nucleotides, nor modified U ribonucleotides other than 1 mΨ and/or Ψ, in the RNA; i.e. 100% U substitution). In particular, the RNA may comprise 1 mΨ and/or Ψ, and neither standard U ribonucleotides nor other modified ribonucleotides (i.e. there are no standard U nucleotides, nor modified ribonucleotides of any type—A, C, G or U substitutable—other than 1 mΨ and/or W, in the RNA; i.e. 100% U substitution with no other modified nucleotides being allowed). The RNA may comprise W, and neither standard U ribonucleotides nor other modified U ribonucleotides (i.e., 100% U substitution with 1). In particular, the RNA may comprise Ψ, and neither standard U ribonucleotides nor other modified ribonucleotides (i.e., 100% U substitution with Ψ with no other modified nucleotides being allowed). In an embodiment, the RNA comprises 1 mΨ, and neither standard U ribonucleotides nor other modified U ribonucleotides (i.e., 100% U substitution with 1 mΨ). In an embodiment, the RNA comprises 1 mΨ, and neither standard U ribonucleotides nor other modified ribonucleotides (i.e., 100% U substitution with 1 mΨ with no other modified nucleotides being allowed).

In some embodiments, the percentage of standard As substituted with A-substitutable modified nucleotide (e.g. those above) is at least: 0.1%, 0.5%, 0.8%, 1%, 2%, 5%, 10%, 20%, 30%, 40%, 50%, 60%, 70%, 80%, 90%, 95%, 96%, 97%, 98%, 99%, 99.5%, or at least 99.9%, or 100%. In some embodiments, the percentage of standard As substituted with m⁶A may be 0.1-5%, in particular 0.5-2%, in particular 0.8-1.2%, such as about 1% (or 1%); in these embodiments the RNA may be circular RNA. Low substitution levels with m⁶A (e.g. 1%) have been shown to inhibit innate immune activation (Chen et al. Mol Cell. 2019 76, 1: 96-109). In some embodiments, the percentage of standard Cs substituted with cytosine-substitutable modified nucleotide (e.g. those above) is at least: 10%, 20%, 30%, 40%, 50%, 60%, 70%, 80%, 90%, 95%, 96%, 97%, 98%, 99%, 99.5%, or at least 99.9%, or 100%. In some embodiments, the percentage of standard Gs substituted with G-substitutable modified nucleotide (e.g. those above) is at least: 10%, 20%, 30%, 40%, 50%, 60%, 70%, 80%, 90%, 95%, 96%, 97%, 98%, 99%, 99.5%, or at least 99.9%, or 100%. In an embodiment, the percentage of standard Us substituted with U-substitutable modified nucleotide (e.g. those above) is at least: 10%, 20%, 30%, 40%, 50%, 60%, 70%, 80%, 90%, 95%, 96%, 97%, 98%, 99%, 99.5%, 99.9%, or 100% e.g. with 1 mΨ and/or P.

Nucleic acids of the present disclosure may be codon optimized e.g., codon optimized RNA. In some embodiments, nucleic acids of the present disclosure may be codon optimised for expression in human cells. Codon optimisation refers to the use of specific codons, which, while not altering the sequence of the expressed protein (given genetic code redundancy), may increase translation efficacy and/or half-life of the nucleic acid.

Therefore, in an embodiment, the RNA is codon-optimised. Codon optimisation may provide an elevated GC content, relative to non-codon optimised RNA encoding the same protein(s). The GC content (the percentage of all ribonucleotides (or, defined alternatively, all “nitrogenous bases”) in the RNA which are G or C) of the RNA may be at least 10%, such as at least 20%, 30%, 35% or at least 40%, 45%, 46%, 47%, 48%, 49%, or at least 50%. The GC content of the RNA may be 10-70%, such as 20-65%, 30-65%, 35-65%, 40-60%, 45-55%, 46-53%, 47-51%, or 48-50%. Codon optimisation may provide an elevated C content relative to non-codon optimised RNA encoding the same protein(s). The percentage of C-optimisable codons in the RNA which have been substituted, as a result of codon optimisation, for a codon with greater C content (while encoding the same amino acid) may be least 30%, such as at least 40%, 50%, 55% or at least 60%, 65%, 66%, 67%, 68%, 69%, 70%, 71%, 72% or at least 72%. The percentage of C-optimisable codons in the RNA which have been substituted, as a result of codon optimisation, for a codon with greater C content (while encoding the same amino acid) may be 30-80%, such as 40-90%, 45-90%, 50-80%, 55-80%, 60-80%, 65-75%, 66-75%, 67-75%, 68-75%, 69-75%, 70-74%, 71-74% or 72-74%

Where the translation of the mRNA is intended to be driven by mammalian UTRs, they may be selected from the 5′ and 3′ UTRs of RNA transcripts of the following genes (i.e. the following human genes): beta-actin, albumin, ATP synthase beta subunit, fibroblast activation protein (“FAP”), H4 clustered histone 15 (“HIST2H4A”), glyceraldehyde-3-phosphate dehydrogenase, heat shock protein family A (Hsp70) member 8 gene, interleukin-2 gene (“IL-2”), and transferrin. In some embodiments, the RNA comprises a 5′ and a 3′ UTR selected from:
SEQ ID NOs: 1 and 2, respectively,
SEQ ID NOs: 3 and 4, respectively,
SEQ ID NOs: 5 and 6, respectively,
SEQ ID NOs: 7 and 8, respectively,
SEQ ID NOs: 9 and 10, respectively,
SEQ ID NOs: 12 and 13, respectively and
RNA sequences at least 70%, 80%, 85%, 90%, 95%, 96%, 98%, 99% or at least 99.5% identical to SEQ ID NO: 1, 3, 5, 7, 9, or 12 (for the 5′ UTR) and RNA sequences at least 70%, 80%, 85%, 90%, 95%, 96%, 98%, 99% or at least 99.5% identical to SEQ ID NO: 2, 4, 6, 8, 10, or 13 (for the 3′ UTR) (in particular, the pairing of 5′ and 3′ UTRs having such identity to SEQ ID NOs: 1 and 2, SEQ ID NOs: 3 and 4, SEQ ID NOs: 5 and 6, SEQ ID NOs: 7 and 8, SEQ ID NOs: 9 and 10, and SEQ ID NOs: 12 and 13, respectively);

RNA sequences according to SEQ ID NOs: 1 and 2, SEQ ID NOs: 7 and 8, SEQ ID NOs: 9 and 10, SEQ ID NOs: 12 and 13 (and RNA sequences having such identity thereto e.g., at least 95% or greater) are more preferred; and RNA sequences according to SEQ ID NO: 1 and 2 (and RNA sequences having such identity thereto e.g., at least 95% or greater) are even more preferred.

Both the 3′ and 5′ UTR may influence expression of the encoded protein through a variety of mechanisms. Without wishing to be bound by this theory, the 5′ UTR may affect the expression of the encoded protein e.g., via pre-initiation complex regulation, closed-loop regulation, upstream open reading frame regulations (i.e. reinitiation), provision of internal ribosome entry sites, and provision of microRNA binding sites. Without wishing to be bound by this theory, the 3′ UTR may affect the expression of the encoded protein e.g., via providing regulation regions that post-transcriptionally influence expression; e.g. influencing translation efficiency, localisation of the RNA, stability of the RNA, polyadenylation, and circularization of the RNA.

In one specific embodiment, the RNA is circular RNA.

The RNA can be prepared by any means known to the person skilled in the art. In an embodiment the RNA is produced by in vitro transcription (IVT). IVT can use a (DNA) template created and propagated in plasmid form in bacteria or created synthetically (for example by gene synthesis and/or polymerase chain-reaction (PCR) engineering methods). For instance, a DNA-dependent RNA polymerase (such as the bacteriophage T7, T3 or SP6 RNA polymerases) can be used to transcribe the replicating RNA from a DNA template. Appropriate capping and poly-A addition reactions can be used as required (although the poly-A tail is usually encoded within the DNA template).

Nucleic acid (especially RNA) by themselves and unprotected, may be degraded by the subject's nucleases and may require a cater to facilitate target cell entry. Accordingly, the present disclosure provides the nucleic acid (e.g., RNA) and a carrier.

The carrier may be lipid-based (e.g., a lipid nanoparticle).

The pharmaceutical composition disclosed herein comprises nucleic acid (e.g., RNA) and lipid carrier particles. Lipid carrier particles provide a means to protect the nucleic acid (e.g., RNA), and deliver it to target cells for protein expression. With a CNE, the nucleic acid (e.g., RNA) which encodes the protein (optionally the protein immunogen) is complexed with a CNE particle, in particular comprising an oil core and a cationic lipid. The cationic lipid can interact with the negatively charged molecule, thereby anchoring the molecule to the emulsion particles. The nucleic acid complexes with the negatively charged molecule of the cationic lipid on the surface of the CNE particle, CNEs do not encapsulate the nucleic acid.

In certain embodiments, the nucleic acid is encapsulated in lipid carrier particles. In a preferred embodiment, nucleic acid (e.g., RNA) is encapsulated in a lipid nanoparticle (LNP). Thus, in a preferred embodiment the lipid carrier particles are lipid nanoparticles. In an embodiment the nucleic acid and lipid carrier particles are nucleic acid containing lipid nanoparticles. In an embodiment the nucleic acid and lipid carrier particles are RNA (e.g., mRNA) containing lipid nanoparticles.

LNPs are non-virion liposome particles within which nucleic acid (e.g., RNA) can be encapsulated. LNP delivery systems and methods for their preparation involves mixing (i) an ethanolic solution of the lipids (ii) an aqueous solution of the nucleic acid and (iii) buffer, followed by mixing, equilibration, dilution and purification. In an embodiment, RNA is encapsulated within the liposomes, and the liposome forms an outer layer around an aqueous RNA-containing core. This encapsulation has been found to protect RNA from RNase digestion.

A plurality of such LNPs will be part of the pharmaceutical composition disclosed herein comprising free and/or encapsulated nucleic acid (e.g., RNA), and in some embodiments the LNPs encapsulate at least: 75%, 76%, 77%, 78%, 89%, 80%, 81%, 82%, 83%, 84%, 85%, 86%, 87%, 88%, 89%, 90%, 91%, 92%, 93%, 94%, 95%, 96%, 97.0%, 97.1%, 97.2%, 97.3%, 97.4%, 97.5%, 97.6%, 97.7%, 97.8%, 97.9%, 98.0%, 98.1%, 98.2%, 98.3%, 98.4%, 98.5%, 98.6%, 98.7%, 98.8%, 98.9%, 99.0%, 99.1%, 99.2%, 99.3%, 99.4%, 99.5%, 99.6%, 99.7%, 99.8%, 99.9%, or at least 100% of the total number of nucleic acid (e.g., RNA) molecules in the composition.

At least 80% of the LNPs in the composition may be 20-200 nm, 40-190 nm, 60-180 nm or, in particular, 80-160 nm in diameter. In a particular embodiment, substantially all, or all, LNPs in the composition are 20-200 nm, 40-190 nm, 60-180 nm or, in particular, 80-160 nm in diameter.

The LNP can comprise multilamellar vesicles (MLV), small uniflagellar vesicles (SUV), or large unilamellar vesicles (LUV).

The amount of nucleic acid (e.g., RNA) per LNP can vary, and the number of individual nucleic acid molecules per LNP can depend on the characteristics of the particle being used. For RNA molecules, in general, an LNP may include 1-500 RNA molecules, e.g. <200, <100, <50, <20, <10, <5, or 1-4. Generally, an LNP includes fewer than 10 different species of RNA e.g. fewer than 5, 4, 3, or 2 different species. In an embodiment, the LNP includes a single RNA species (i.e. all RNA molecules in the particle have the same sequence).

LNPs according to the present disclosure may be formed from a single lipid (e.g. a cationic lipid) or, in particular, from a mixture of lipids. In particular, the mixture comprises various classes of lipids, such as:
(a) a mixture of cationic lipids and sterols,
(b) a mixture of cationic lipids and neutral lipids,
(c) a mixture of cationic lipids and polymer-conjugated lipids,
(d) a mixture of cationic lipids, sterols and polymer-conjugated lipids, or
(e) a mixture of cationic lipids, neutral lipids and polymer-conjugated lipids;
(f) a mixture of cationic lipids, sterols and neutral lipids;
   or preferably:
(g) a mixture of cationic lipids, neutral lipids, sterols and polymer-conjugated lipids.
   Further classes of lipids, such as anionic lipids, may also be present in a mixture of lipids.

In an embodiment the LNPs comprise a cationic lipid, a neutral lipid and a PEGylated lipid.

The cationic lipid may have a pKa of 5.0-10.0, 5.0-9.0, 5.0-8.5, 5.0-8.0, 5.0-7.9, 5.0-7.8, 5.0-7.7, or 5.0-7.6. The pKa of the cationic lipid is distinct to the pKa of the LNP as a whole (sometimes called “apparent pKa”). pKa may be determined via any well-known method, such as via a toluene nitrosulphonic acid (TNS) fluorescence assay or acid base titration.

In an embodiment, the cationic lipid is an ionizable amino lipid. In an embodiment the cationic lipid is unsaturated.

In an embodiment, the cationic lipid comprises a tertiary or quaternary amine group. In an embodiment, the cationic lipid comprises a tertiary amine group. Exemplary cationic lipids comprising tertiary amine groups include: 1,2-dilinoleyoxy-3-(dimethylamino)acetoxypropane (DLin-DAC), 1,2-dilinoleyoxy-3morpholinopropane (DLin-MA), 1,2-dilinoleoyl-3-dimethylaminopropane (DLinDAP), 1,2-dilinoleylthio-3-dimethylaminopropane (DLin-S-DMA), 1-linoleoyl-2-linoleyloxy-3dimethylaminopropane (DLin-2-DMAP), 1,2-dilinoleyloxy-3-trimethylaminopropane chloride salt (DLin-TMA.Cl), 1,2-dilinoleoyl-3-trimethylaminopropane chloride salt (DLin-TAP.Cl), 1,2-dilinoleyloxy-3-(N-methylpiperazino)propane (DLin-MPZ), 3-(N,Ndilinoleylamino)-1,2-propanediol (DLinAP), 3-(N,N-dioleylamino)-1,2-propanediol (DOAP), 1,2-dilinoleyloxo-3-(2-N,N-dimethylamino)ethoxypropane (DLin-EG-DMA), and 2,2-dilinoleyl-4-dimethylaminomethyl[1,3]-dioxolane (DLin-K-DMA), 2,2-dilinoleyl-4-(2-dimethylaminoethyl)[1,3]-dioxolane (DLin-KC2-DMA); dilinoleyl-methyl-4-dimethylaminobutyrate (DLin-MC3-DMA); or MC3 (see, e.g. US20100324120).

In some embodiments, the cationic lipid has the structure of lipid RV28, RV31, RV33, RV37, RV39, RV42, RV44, RV73, RV75, RV81, RV84, RV85, RV86, RV88, RV91, RV92, RV93, RV94, RV95, RV96, RV97, RV99, or RV101, as disclosed in WO 2021/038508. In a further embodiment, the cationic lipid has the structure:

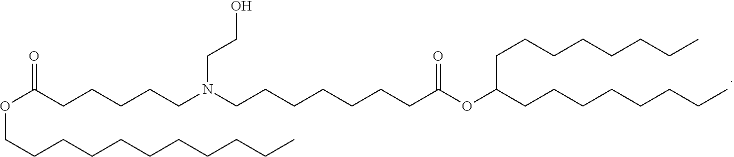 [SMILES: CCCCCCCCCCCOC(=O)CCCCCN(CCO)CCCCCCCC(=O)OC(CCCCCCCC)CCCCCCCC]

In an embodiment, the LNP comprises a cationic lipid and the cationic lipid is RV39. In an embodiment, RV39 is 2,5-bis((9Z,12Z)-octadeca-9,12-dien-1-yloxy)benzyl 4-(dimethylamino)butanoate. In an embodiment, RV39 has the structure:

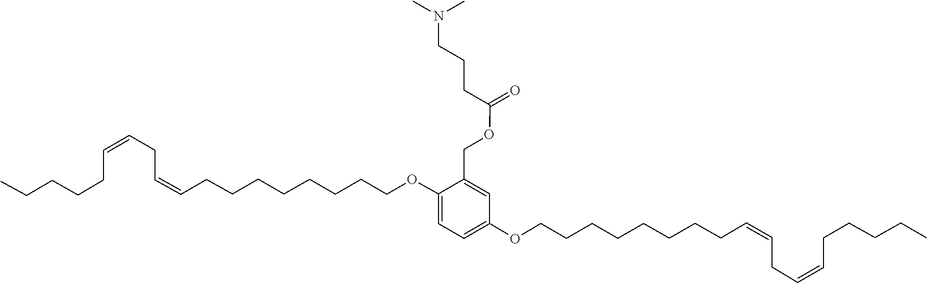 [SMILES: CCCCC/C=C\C/C=C\CCCCCCCCOc1ccc(OCCCCCCCC/C=C\C/C=C\CCCCC)c(COC(=O)CCCN(C)C)c1]

In another embodiment, the cationic lipid has the structure:

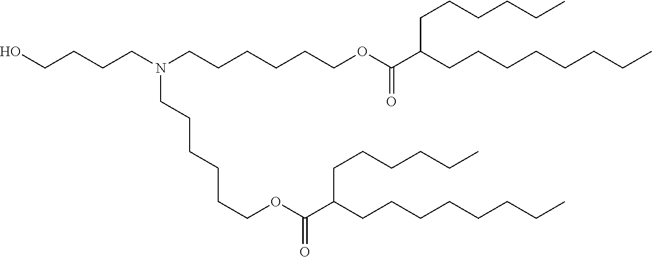 [SMILES: CCCCCCCCC(CCCCCC)C(=O)OCCCCCCN(CCCCO)CCCCCCOC(=O)C(CCCCCC)CCCCCCCC]

In another embodiment, the cationic lipid has the structure:

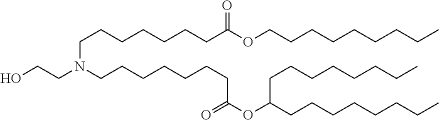 [SMILES: CCCCCCCCCOC(=O)CCCCCCCN(CCO)CCCCCCCC(=O)OC(CCCCCCCC)CCCCCCCC]

The lipids in the LNP may comprise (in mole %) 20-80, 25-75, 30-70, 35-65%, 30-60, 35-55 or 35-50% cationic lipid; such as about 40% (or 40%), about 42% (or 42%), about 44% (or 44%), about 46% (or 46%) or about 48% (or 48%) cationic lipid. The lipids in the LNP may comprise (in mole %) at least 20, 25 or at least 35%, or at least 40% cationic lipid. The lipids in the LNP may comprise (in mole %) no more than 80, 70 or no more than 60% or no more than 50% cationic lipid.

The molar ratio of protonatable nitrogen atoms in the LNP's cationic lipids to phosphates in the nucleic acid (a.k.a “N:P” ratio), may be in the range of (including the endpoints) 1:1-20:1, 2:1-10:1, 3:1-9:1, or 4:1-8:1, for example within the range 4.5:1-7.5:1, 4.5:1-6.5:1 or 5.0:1-6.5:1. In an embodiment, the N:P ratio is between 7:1 and 9:1 e.g. about 8:1.

In an embodiment, the polymer-conjugated lipid is a PEGylated lipid. In an LNP, the PEGs of such PEGylated lipids may have average molecular weight of 0.5-11.0 kDa; such as 0.5-8.0, 0.8-8.0, 0.8-7.0, 0.8-6.0, 0.8-5.0, 0.8-4.0, 1.0-4.0 or 1.0-3.5, 1.0-3.0, 1.2-2.8, 1.4-2.6, 1.5-2.5, 1.6-2.4, or 1.7-2.3, 1.8-2.2 or 1.9-2.1 kDa. In an embodiment the PEGs of such PEGylated lipids may have an average molecular weight of about 2.0 (or 2.0 kDa). The average molecular weight of such PEGs may be expressed as the median molecular weight. Alternatively, in an LNP, at least 80% of the PEGs of such PEGylated lipids may have molecular weight of 0.5-11.0 kDa; such as 0.5-8.0, 0.8-8.0, 0.8-7.0, 0.8-6.0, 0.8-5.0, 0.8-4.0, 1.0-4.0, 1.0-3.5, 1.0-3.0, 1.2-2.8, 1.4-2.6, 1.5-2.5, 1.6-2.4, or 1.7-2.3, 1.8-2.2 or 1.9-2.1 kDa.

The PEGylated lipid may have the structure:

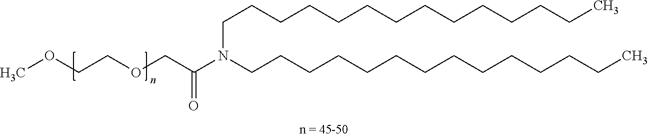

Exemplary PEGylated lipids include 2-[(polyethylene glycol)-2000]-N,N-ditetradecylacetamide and 1,2-dimyristoyl-rac-glycero-3-methoxypolyethylene glycol-2000, 1,2-dimyristoyl-sn-glycero-2-phosphoethanolamine-N-[methoxy(polyethylene glycol)] and 1,2-dimyristoyl-rac-glycerol-3-methoxypolyethylene glycol. In an embodiment, the PEGylated lipid is 2-[(polyethylene glycol)-2000]-N,N-ditetradecylacetamide or 1,2-dimyristoyl-rac-glycero-3-methoxypolyethylene glycol-2000. In an embodiment, the PEGylated lipid prevents particle aggregation and increases circulation lifetime. In an embodiment the PEGylated lipid has the structure:

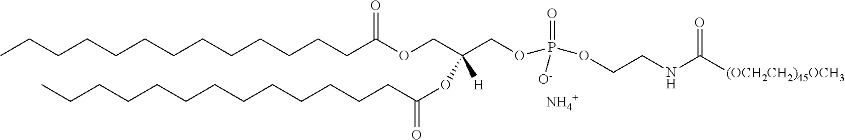

In an embodiment the PEGylated lipid is present from 0.5% to 5% (w/v). In an embodiment the PEGylated lipid is present from 1% to % (w/v), optionally 2% (w/v). The lipids in the LNP may comprise (in mole %) 0.1-8.0, 0.4-7.0, 0.6-6.0, 0.8-4.0 or 0.8-3.5% or 1.0-3.0% polymer-conjugated lipid (e.g., PEGylated lipid); such as about 1.0 (or 1.0%), about 1.5% (or 1.5%), about 2.0% (or 2.0%) or about 2.5% (or 2.5%) polymer-conjugated lipid (e.g., PEGylated lipid). The lipids in the LNP may comprise (in mole %) at least 0.1, 0.5 or at least 0.8%, or at least 1% polymer-conjugated lipid (e.g., PEGylated lipid). The lipids in the LNP may comprise (in mole %) no more than 8.0, 6.0 or 4.0% or no more than 3.0% polymer-conjugated lipid (e.g., PEGylated lipid).

In an embodiment, the neutral lipid is 1,2-distearoyl-sn-glycero-3-phosphocholine (DSPC) or 1,2-dioleoyl-sn-glycero-3-phosphoethanolamine (DOPE), although other neutral lipids available to the skilled person may also be used. In an embodiment the neutral lipid is DSPC. In an embodiment the neutral lipid has the structure:

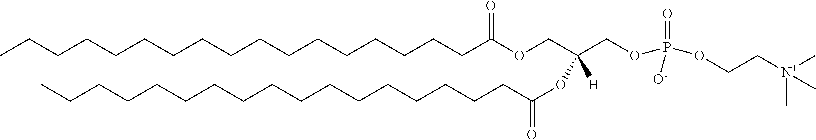 [SMILES: CCCCCCCCCCCCCCCCCC(=O)OC[C@H](COP(=O)([O-])OCC[N+](C)(C)C)OC(=O)CCCCCCCCCCCCCCCCC]

The lipids in the LNP may comprise (in mole %) 0-15.0, 0.1-15.0, 2.0-14.0, 5.0-13.0, 6.0-12.0 or 7.0-11.0%, 8.0-11.0% or 9.0-11.0% neutral lipid, such as about 9.4% (or 9.4%), about 9.6% (or 9.6%), about 9.8% (or 9.8%) or about 10.0% (or 10%) neutral lipid. The lipids in the LNP may comprise (in mole %) at least 0.1, 5.0 or at least 7.0%, or at least 8.0% or at least 9.0% neutral lipid. The lipids in the LNP may comprise (in mole %) no more than 15.0, 13.0 or no more than 12.0%, or no more than 11.0% neutral lipid. Neutral lipid may also herein be referred to as zwitterionic lipids.

Exemplary sterols include cholesterol, cholesterol sulfate, desmosterol, stigmasterol, lanosterol, 7-dehydrocholesterol, dihydrolanosterol, symosterol, lathosteriol, 14-demethyl-lanosterol, 8(9)-dehydrocholesterol, 8(14)-dehydrocholesterol, 14-demethyl-14-dehydrolanosterol (FF-MAS), diosgenin, dehydroepiandrosterone sulfate (DHEA sulfate), dehydroepiandrosterone, sitosterol, lanosterol-95, 4,4-dimethyl(d6)-cholest-8(9), 14-dien-3β-ol (dihydro-FF-MAS-d6), 4,4-dimethyl(d6)-cholest-8(9)-en-3β-ol (dihydro T-MAS-d6), zymostenol, sitostanol, campestanol, camperstanol, 7-dehydrodesmosterol, pregnenolone, 4,4-dimethyl-cholest-8(9)-en-3β-ol (dihyrdro T-MAS), Δ5-avensterol, brassicasterol, dihydro FF-MAS, 24-methylene cholesterol, oxysterols, deuterated sterols, fluorinated sterols, sulfonated sterols, phosphorylated sterols, A-ring substituted sterols, cholest-5-ene-3ß,4ß-diol, 5α-cholestan-3ß-ol, 4-cholesten-3-one, cholesta-8(9),24-dien-3-one, cholesta-8(9),24-dien-3-one, 2,2,3,4,4-pentadeuterio-5a-cholestan-3ß-ol, cholesteryl phosphocholine, cholesteryl-d7 pentadecanoate, cholesteryl-d7 palmitate, B-ring substituted sterols, cholestanol, 5ß,6ß-epoxy-d7, 3ß-hydroxy-5-cholestene-7-one, 6α-hydroxy-5α-cholestane, cholestanol, 5α,6α-epoxy, cholest-5-en-3ß,7α-diol, cholest-5-en-3ß,7ß-diol, cholestanol, 5α,6α-epoxy-d7, Δ5,7-cholesterol, cholesta-5,8(9)-dien-3ß-ol, cholesta-5,8(14)-dien-3ß-ol, 7α-hydroxy-4-cholesten-3-one, zymostenol-d7, zymostenol, 7-dehydrodesmosterol, 3b,5a-dihydroxy-cholestan-6-one, D-ring substituted sterols, 3ß-hydroxy-5α-cholest-8(14)-en-15-one, 3ß-hydroxy-5α-cholestane-15-one, 5α-cholest-8(14)-ene-3ß,15α-diol, 5α-cholest-8(14)-ene-3ß,15ß,-diol, lanosterol-95, 5α-7,24-cholestadiene, 14-dehydro zymostenol, ergosta-5,7,9(11),22-tetraen-3ß-ol, cholest-5-ene-3ß,25-diol, cholest-(25R)-5-ene-3ß,27-diol, 24(R/S),25-epoxycholesterol, 24(S),25-epoxycholesterol, 24(R/S),25-epoxycholesterol-d6, cholest-5-ene-3ß,22(S)-diol, cholest-5-ene-3ß,22(R)-diol, cholest-5-ene-3ß,24(S)-diol, cholest-5-ene-3ß,24(R)-diol, 27-hydroxy-4-cholesten-3-one, campestanol, N,N-dimethyl-3ß-hydroxycholenamide, 25,27-dihydroxycholesterol, N,N-dimethyl-3ß-hydroxycholenamide, 25,27-dihydroxycholesterol, 5-cholestene-3ß,20α-diol, 24S,25-epoxy-5α-cholest-8(9)-en-3β-ol, 24(S/R),25-epoxylanost-8(9)-en-3ß-ol, 7-keto-27-hydroxycholesterol, 7α,27-dihydroxy-4-cholesten-3-one, 7α,27-dihydroxycholesterol, 7ß,27-dihydroxycholesterol, 5α,6ß-dihydroxycholestanol, 7α,25-dihydroxycholesterol, 7ß,25-dihydroxycholesterol, 7α,24(S)-dihydroxycholesterol, 7α,24(S)-dihydroxy-4-cholesten-3-one, 7-keto-25-hydroxycholesterol, 7α,24S,27-trihydroxycholesterol, dihydrotestosterone, testosterone, estrone, estrogen, estradiol, corticosterone, cortisol, or 24S,27-dihydroxycholesterol.

In an embodiment the LNPs further comprise a sterol. In an embodiment the LNPs further comprise a cholesterol-based lipid. In an embodiment the LNPS further comprise cholesterol. In an embodiment the cholesterol stabilizes the nanoparticles by adding mechanical strength and elasticity and increases lipid packing density compared to an LNP lacking cholesterol.

In an embodiment the cholesterol has the structure:

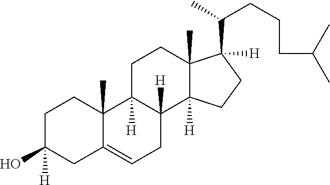

In an embodiment the cholesterol is present from 40% to 60% (w/v), optionally 45% to 55%, optionally 45% to 50% (w/v). In an embodiment, the lipids in the LNP may comprise (in mole %) 20-80, 25-80, 30-70, 30-60, 35-60, 40-60%, 40-50% or 41-49% sterol; such as about 42% (or 42%), about 43% (or 43%), about 44% (or 44%), about 46% (or 46%), or about 48% (or 48%) sterol. The lipids in the LNP may comprise (in mole %) at least 20, 30 or at least 35%, or at least 40% or at least 41% sterol. The lipids in the LNP may comprise (in mole %) no more than 80, 70 or no more than 60%, or no more than 50% sterol.

The lipids in the LNP may have the following mole % in combination: 30-60% cationic lipid (such as 35-55%, or 35-45%), 35-70% sterol (such as 40-55%, or 41-49%), 0.8-4.0% polymer-conjugated lipid (such as 0.8-3.5%, or 1.0-3.0%), and 0-15% neutral lipid (such as 6.0-12.0% or 8.0-11.0%).

In a particular embodiment the LNP comprises the cationic lipid RV39, the neutral lipid DSPC, cholesterol and a PEGylated lipid. In an embodiment the LNP comprises the cationic lipid RV39, the neutral lipid DSPC, cholesterol and a PEGylated lipid wherein the cationic lipid is present at a molar percentage of 40%±5%, the neutral lipid is present at a molar percentage of 10%±5%, cholesterol is present at 48% (w/v)±5% and the PEGylated lipid is present at 2% (w/v)±0.5%.

Such LNPs encapsulating nucleic acids (e.g., RNA) may be formed by admixing a first solution comprising the nucleic acids with a second solution comprising lipids which form the LNP. The admixing may be performed by any suitable means available to the skilled person, e.g., a T-mixer, microfluidics, or an impinging jet mixer. Admixing may be followed by filtration. The filtration may be performed by any suitable means available to the skilled person, e.g., tangential-flow filtration or cross-flow filtration.

Accordingly, in a further aspect, the present disclosure provides a method of preparing an LNP encapsulating a nucleic acid (e.g., RNA) encoding at least one protein, optionally a protein immunogen, comprising admixing a first solution comprising the nucleic acid and a second solution comprising lipids which form the LNP (e.g. using the means as set out in the foregoing paragraph); and optionally filtering the obtained admixture (e.g. using the means as set out in the foregoing paragraph).

As already described, preferably the nucleic acid is RNA, optionally mRNA. Furthermore, in an embodiment the lipid carrier particles are lipid nanoparticles. As such, the first aspect provides a pharmaceutical composition said pharmaceutical composition comprising RNA-LNPs (e.g., mRNA-LNPs), wherein the pharmaceutical composition is lyophilised and wherein either
(a) the percentage of RNA (e.g., mRNA) that is encapsulated within LNP is greater than 75% of the total RNA (e.g., mRNA) in the pharmaceutical composition; and/or
(b) the percentage of RNA (e.g., mRNA) that remains encapsulated within LNP is reduced by less than 15% compared to the percentage of RNA (e.g., mRNA) that is encapsulated within the LNP in the pharmaceutical composition prior to lyophilisation.

The pharmaceutical composition of the present disclosure is a pharmaceutical formulation comprising a plurality of constituents. In an embodiment, the pharmaceutical composition further comprises sucrose. In an embodiment the pharmaceutical composition comprises a nucleic acid, a lipid carrier particle and sucrose.

The pharmaceutical composition of the present aspect is lyophilised. However, the pharmaceutical composition disclosed herein is lyophilised from an aqueous composition (i.e., an aqueous pre-lyophilisation solution) comprising said nucleic acid and lipid carrier particles. Said aqueous composition is formulated for stable lyophilisation of the pharmaceutical composition comprising nucleic acid and lipid carrier particles.

In an embodiment the aqueous composition comprises an amorphous sugar. The amorphous sugar is considered to be a cryoprotectant and/or a lyoprotectant. The term “cryoprotectant” refers to a class of excipients which prevents freeze damage of what is being frozen. In the present disclosure the term cryoprotectant means a class of excipients that prevent freeze damage to the nucleic acid and lipid carrier particles. The term “lyoprotectant” refers to a class of excipients that prevent drying damage of what is being freeze-dried. In the present disclosure the term lyoprotectant means a class of excipients that prevent drying damage to the pharmaceutical composition comprising nucleic acid and lipid carrier particles.

In an embodiment the amorphous sugar is selected from sucrose, trehalose, mannose, mannitol, raffinose, lactitol, lactobionic acid, glucose, maltulose, iso-maltulose, lactulose, maltose, lactose, isomaltose, maltilol, palatinit, stachyose, melezitose, dextran, or a combination thereof. In one embodiment, the amorphouse sugar is selected from sucrose, trehalose, lactose, raffinose, dextran and combinations thereof. In one embodiment, the amorphous sugar is selected from sucrose or trehalose. In a preferred embodiment the amorphous sugar is sucrose.

In an embodiment the aqueous composition comprises an amorphous sugar, wherein said amorphous sugar is sucrose. In an embodiment, the aqueous composition comprises sucrose in a concentration greater than or equal to 5% (w/v). In an embodiment the aqueous composition comprises sucrose in a concentration of between 5% and 30% (w/v), 5% and 20% (w/v) or 5% and 10% (w/v). In an embodiment sucrose is present in the aqueous composition in a total amount of less than 30% (w/v), less than 28% (w/v), less than 26% (w/v), less than 24% (w/v), less than 22% (w/v), less than 20% (w/v), less than 18% (w/v), less than 16% (w/v) or less than 14% (w/v), less than 12% (w/v), less than 10% (w/v), or less than 8% (w/v), In an embodiment, the aqueous composition comprises sucrose in a concentration of 7.5% (w/v).

In a further embodiment the pharmaceutical composition further comprises salt. As used herein “salt” refers to ionic compounds that result from the neutralization reaction of an acid and a base, compose of a related number of cations and anions such that the product is without net charge, for example sodium chloride. The component ions can either be inorganic or organic and can be monoatomic or polyatomic. In an embodiment the salt is sodium chloride. Accordingly, in an embodiment the pharmaceutical composition of the first aspect further comprises a salt, optionally sodium chloride.

In an embodiment the aqueous composition further comprises sodium chloride. In an embodiment the aqueous composition comprises sodium chloride in an amount less than 50 mM, less than 40 mM, less than 30 mM, less than 20 mM, less than 15 mM, less than 10 mM or less than 7.5 mM. In an embodiment the aqueous composition comprises sodium chloride in a concentration of between 0.1 and 50 mM, 0.5 and 40 mM, 1 and 30 mM, 1 and 25 mM, 1 and 10 mM, 1 and 7.5 mM or 2.5 and 7.5 mM. In an embodiment the aqueous composition comprises sodium chloride in a concentration of between 1 and 20 mM, 1 and 10 mM, 2 and 8 mM or 4 and 6 mM. In an embodiment the aqueous composition comprises sodium chloride at a concentration of 5 mM±0.5 mM (or 5 mM). In an embodiment, the composition is not completely devoid of salt (e.g., not completely devoid of sodium chloride).

For the purposes of defining ranges, the term “between” as used herein is considered to include the end points of the range. For example, when sodium chloride is said to be present in an amount between 1 and 7.5 mM, those cases where sodium chloride is present at a concentration of 1 mM or 7.5 mM are included.

The aqueous composition (and thus also the lyophilised pharmaceutical composition) may further comprise a surfactant. The surfactant may act to decrease the surface tension and may also increase the solubility of other formulation components. In an embodiment the aqueous composition further comprises a surfactant wherein the surfactant is selected from poloxamer surfactants (e.g., poloxamer 188), polysorbate surfactants (e.g., polysorbate 80 and/or polysorbate 20), octoxinal surfactants, polidocanol surfactants, polyoxyl stearate surfactants, polyoxyl castor oil surfactants, N-octyl-glucoside surfactants, macrogol 15 hydroxy stearate, and combinations thereof. In an embodiment, the surfactant is selected from poloxamer surfactants (e.g., poloxamer 188), polysorbate surfactants (e.g., polysorbate 80 and/or polysorbate 20), in particular polysorbate surfactants such as polysorbate 80. In an embodiment, the aqueous composition comprises a surfactant wherein the surfactant is present in an amount of at least 0.001% (w/v), at least 0.005% (w/v), at least 0.01% (w/v), and/or up to 0.5% (w/v). In an embodiment the surfactant is present in an amount less than 0.25% (w/v) or less than 0.1% (w/v). In another embodiment, the surfactant is present in an amount of 0.02% (w/v). According to specific embodiments, the surfactant is polysorbate 80 or poloxamer 188 present in the aqueous mixture in an amount between 0.005% and 0.5% (w/v), such as about 0.02% (w/v).

In a further embodiment, the aqueous composition (and thus also the lyophilised pharmaceutical composition) further comprises a buffer. The pH is typically adjusted in view of the therapeutic components of the composition. Suitably, the pH of the aqueous composition is at least 6, at least 6.5, at least 7 or at least 7.5. Alternatively stated, the pH of the aqueous composition may be less than 10, less than 9.5, less than 9 or less than 8.5. In other embodiments, pH of the aqueous composition is between 6 and 10, between 7 and 9.5, between 7.5 and 9.5, or, about 8, for example 8±1 or 8±0.5. An appropriate buffer may be selected from Tris, succinate, borate, Tris-maleate, lysine, histidine, glycine, glycylglycine, citrate, carbonate, phosphate, or combinations thereof. In one embodiment, the aqueous composition further comprises a buffer and the buffer is Tris, succinate, or borate. In a further embodiment, the buffer is Tris. In a particular embodiment, the aqueous composition further comprises a Tris buffer and wherein the pH of the aqueous composition is 8±0.5

In an embodiment, the buffer is present in the aqueous composition in an amount of at least 0.5 mM, at least 1 mM, at least 2 mM or at least 5 mM. In an embodiment, the buffer is present in the aqueous composition in an amount of less than 50 mM, less than 40 mM, or less than 30 mM. For example, the buffer may be present in the aqueous composition in an amount of 0.5 mm to 50 mM, 1 mM to 50 mM or 2 mM to 30 mM. In one embodiment, the buffer is present in the aqueous composition in an amount of 20±2 mM. According to specific embodiments, the buffer is Tris, present in the aqueous composition in an amount between 15 and 25 mM, optionally 20 mM.

In an embodiment, the aqueous composition (and thus the lyophilised pharmaceutical composition) also comprises histidine in an amount of up to or about 20 mM, such as at a concentration of about 10 mM. According to further embodiments, the composition also comprises bivalent metal ions, such as Mg²⁺, Ca²⁺, or Mg²⁺ or Ca²⁺ in the form of a salt, such as MgCI₂, CaCI₂ or MgSO₄. In one embodiment the bivalent metal ion is Mg2+. Typical amounts wherein the bivalent metal ions are present in the aqueous mixture are between 0.5 and 10 mM, such as 1 or 2 mM, or 1 mM in particular.

In an embodiment the pharmaceutical composition of the first aspect is lyophilised and comprises a residual moisture content of between 0.25 and 3% (w/w), between 0.5 and 2% (w/w), between 0.5 and 1.25% (w/w), or between 0.5 and 1% (w/w). In an embodiment the residual moisture content is determined using the gravimetric, Karl Fischer or thermogravimetric methods, for example those methods disclosed in May et al 1982, Journal of Biological Standardization, vol 10, issue 3. In an embodiment the residual moisture content is determined by the Karl Fischer method, for example as described in Example 1 herein.

In a second aspect there is provided a vaccine comprising the pharmaceutical composition of the first aspect. In an embodiment, the lyophilised pharmaceutical composition of the first aspect is first reconstituted and then administered to a subject as a vaccine. Thus, in an embodiment the vaccine of the second aspect comprises the lyophilised pharmaceutical composition of the first aspect wherein said lyophilised pharmaceutical composition has been reconstituted. In an embodiment the vaccine does not comprise an adjuvant. In an embodiment the vaccine further comprises an adjuvant.

In a third aspect there is provided a method of reconstituting the pharmaceutical composition of the first aspect, comprising adding a sterile aqueous reconstitution solution to the pharmaceutical composition and reconstituting the pharmaceutical composition. In one embodiment, an aqueous sterile reconstitution solution such as sterile water for injection is introduced into a vessel (such as a sterile vial) containing the lyophilised composition in an amount sufficient to provide the desired concentration of the various components of the vaccine in the injectable solution. In an embodiment the vessel will have enough the lyophilised pharmaceutical composition for a single dose. In another embodiment, the container will have enough of the lyophilised pharmaceutical composition for multiple doses, such as 2-10 doses or 3-8 doses or 4-8 doses or 5-6 doses. In an embodiment, the aqueous sterile reconstitution solution is introduced into the vessel and the vessel is shaken. Following this the vessel is inspected for expected and desired visual properties, such as degree of transparency, color, etc. The solution is then ready for being withdrawn from the vial by techniques known in the art, such as by inserting a syringe needle into the vial and withdrawing an appropriate amount of injectable solution into the syringe. In a separate embodiment, the lyophilised pharmaceutical composition and the liquid for reconstitution are present in separate chambers of a dual chamber syringe and are then combined prior to administration. Although sterile water for injection may be a suitable liquid for reconstitution, other aqueous solutions such as buffers containing other additive, excipients and/or adjuvants may be included in the liquid for reconstitution if these other additives, excipients and/or adjuvants are not present, or are not present in sufficient quantities, in the lyophilised composition. Reconstitution can take place at any suitable temperature but will typically take place at room temperature.

The lyophilised pharmaceutical composition or vaccine as disclosed herein can be part of a kit that contains the lyophilised pharmaceutical composition or vaccine of the present disclosure in one container and a liquid to be used for reconstitution in another container. In such a situation, after reconstitution, the resulting formulation will have acceptable properties for injection, for example acceptable and physiologically relevant temperature, pH, osmolarity etc.

Accordingly, in a fourth aspect there is provided a kit comprising the pharmaceutical composition of the first aspect or the vaccine of the second aspect, the kit comprising a first container comprising the pharmaceutical composition of the first aspect or the vaccine of the second aspect and a second container comprising a sterile aqueous reconstitution solution. In an embodiment the sterile aqueous reconstitution solution is water, saline solution (e.g., 0.4%±0.1% or 0.9%±0.1% sodium chloride) or a liquid adjuvant solution. In an embodiment, said kit further comprises a sterile needle for injecting the pharmaceutical composition of the first aspect or vaccine of the second aspect. Instructions for reconstitution at the clinic site and for administration of the reconstituted vaccine composition to the subject will be included in, on or associated with the various components of the kit. In an embodiment the kit will comprise one unit dose i.e., one first container, one second container and one sterile needle thus enabling administration of the pharmaceutical composition or vaccine to one subject. In an alternative embodiment the kit comprises a plurality (e.g., greater than 5, greater than 10, greater than 50, greater than 100 or greater than 500) of unit doses i.e., greater than 5, 10, 50, 100 or 500 first containers, greater than 5, 10, 50, 100 or 500 second containers and greater than 5, 10, 50, 100 or 500 sterile needles. In an embodiment, the kit will always comprise the same number of first containers, second containers and sterile needles.

Also contemplated is a kit comprising the pharmaceutical composition of the first aspect or the vaccine of the second aspect, said kit comprising a device having at least two chambers, wherein a first chamber comprises the pharmaceutical composition of the first aspect or the vaccine of the second aspect and a second container comprises a sterile aqueous reconstitution solution. In an embodiment, said device is a dual-chamber syringe.

In a fifth aspect there is provided a method for producing the pharmaceutical composition of the first aspect said method comprising
i) increasing the surface area to volume ratio of an aqueous composition comprising nucleic acid and lipid carrier particles,
ii) subjecting the aqueous composition to freezing conditions sufficient to freeze the aqueous composition, and
iii) drying the frozen composition.

In an embodiment, step i), step ii) and step iii) occur sequentially. In an embodiment step i) and step ii) overlap. In an embodiment step i) and step ii) occur substantially simultaneously. In an embodiment, step i) and step ii) occur substantially simultaneously wherein substantially simultaneously means that increasing the surface area to volume ratio of the aqueous composition occurs whilst subjecting the aqueous composition said freezing conditions. It is however important that when step i) and step ii) overlap or occur substantially simultaneously, the freezing conditions sufficient to freeze the aqueous composition must not be achieved prior to the surface area to volume ratio of the aqueous composition being increased, otherwise, the composition will transition from an aqueous to a solid state thus preventing the increase in surface area to volume ratio. Accordingly, in an embodiment there is provided a method for producing the pharmaceutical composition of the first aspect said method comprising
i) increasing the surface area to volume ratio of an aqueous composition comprising nucleic acid and lipid carrier particles,
ii) during the increasing the surface area to volume ratio of the aqueous composition according to step i) subjecting the aqueous composition to freezing conditions sufficient to freeze the aqueous composition, and
iii) drying the frozen composition.

To avoid undue repetition the features of the pharmaceutical composition of the first aspect shall not be repeated with respect to the method of the fifth aspect. However, in brief, the method of the fifth aspect provides a method for producing a pharmaceutical composition said pharmaceutical composition comprising a nucleic acid and lipid carrier particles wherein the pharmaceutical composition is lyophilised and wherein either (a) the percentage of nucleic acid that is encapsulated within the lipid carrier particles is greater than 75% of the total nucleic acid in the pharmaceutical composition; and/or (b) the percentage of nucleic acid that is encapsulated within the lipid carrier particles is reduced by less than 15% compared to the percentage of nucleic acid that is encapsulated within the lipid carrier particles in the pharmaceutical composition prior to lyophilisation. said method comprising i) increasing the surface area to volume ratio of an aqueous composition comprising nucleic acid and lipid carrier particles, ii) subjecting the aqueous composition to freezing conditions sufficient to freeze the aqueous composition, and iii) drying the frozen composition.

In an embodiment, the method of the fifth aspect provides a method for producing a pharmaceutical composition said pharmaceutical composition comprising RNA and LNPs (e.g. mRNA-LNPs) wherein the pharmaceutical composition is lyophilised and wherein either (a) the percentage of RNA (e.g. mRNA) that is encapsulated within the LNPs is greater than 75% of the total RNA in the pharmaceutical composition; and/or (b) the percentage of RNA (e.g. mRNA) that is encapsulated within the LNPs is reduced by less than 15% compared to the percentage of RNA that is encapsulated within the LNPs in the pharmaceutical composition prior to lyophilization, said method comprising i) increasing the surface area to volume ratio of an aqueous composition comprising RNA (e.g mRNA) and LNPs, ii) subjecting the aqueous composition to freezing conditions sufficient to freeze the aqueous composition iii) drying the frozen composition. In an embodiment, the method of the fifth aspect provides a method for producing a pharmaceutical composition said pharmaceutical composition comprising RNA (e.g., mRNA) containing LNPs wherein the pharmaceutical composition is lyophilised and wherein either (a) the percentage of mRNA that is encapsulated within the LNPs is greater than 75% of the total mRNA in the pharmaceutical composition; and/or (b) the percentage of mRNA that remains encapsulated within the LNPs is reduced by less than 15% compared to the percentage of mRNA that is encapsulated within the LNPs in the pharmaceutical composition prior to lyophilisation said method comprising i) increasing the surface area to volume ratio of an aqueous composition comprising mRNA containing LNPs, ii) subjecting the aqueous composition to freezing conditions sufficient to freeze the aqueous composition and iii) drying the frozen composition. In an embodiment the mRNA is self-amplifying mRNA.

In an embodiment, the aqueous composition comprising nucleic acid and lipid carrier particles is an aqueous composition comprising RNA (e.g., mRNA) and LNPs. In an embodiment, the aqueous composition further comprises an amorphous sugar. In an embodiment the amorphous sugar is selected from sucrose, trehalose, mannose, mannitol, raffinose, lactitol, lactobionic acid, glucose, maltulose, iso-maltulose, lactulose, maltose, lactose, isomaltose, maltilol, palatinit, stachyose, melezitose, dextran, or a combination thereof. In an embodiment the aqueous composition comprises an amorphous sugar, wherein said amorphous sugar is sucrose. In an embodiment the aqueous composition comprises sucrose in a concentration of between 5% and 30% (w/v), 5% and 20% (w/v) or 5% and 15% (w/v). In an embodiment sucrose is present in the aqueous composition in a total amount of less than 30% (w/v), less than 28% (w/v), less than 26% (w/v), less than 24% (w/v), less than 22% (w/v), less than 20% (w/v), less than 18% (w/v), less than 16% (w/v) or less than 14% (w/v), less than 12% (w/v), less than 10% (w/v), or less than 8% (w/v), In an embodiment, the aqueous composition comprises sucrose in a concentration of 7.5% (w/v).

In a further embodiment the aqueous composition further comprises salt. As used herein “salt” refers to ionic compounds that result from the neutralization reaction of an acid and a base, compose of a related number of cations and anions such that the product is without net charge, for example sodium chloride. The component ions can either be inorganic or organic and can be monoatomic or polyatomic. In an embodiment the salt is sodium chloride. In an embodiment the aqueous composition comprises sodium chloride in an amount less than 50 mM, less than 40 mM, less than 30 mM, less than 20 mM, less than 15 mM, less than 10 mM or less than 7.5 mM. In an embodiment the aqueous composition comprises sodium chloride in a concentration of between 0.1 and 50 mM, 0.5 and 40 mM, 1 and 30 mM, 1 and 25 mM, 1 and 10 mM, 1 and 7.5 mM or 2.5 and 7.5 mM. In an embodiment the aqueous composition comprises sodium chloride in a concentration of between 1 to 20 mM, 1 to 10 mM, 2 to 8 mM or 4 to 6 mM. In an embodiment the aqueous composition comprises sodium chloride at a concentration of 5 mM±0.5 mM (or 5 mM).

The aqueous composition may further comprise a surfactant. In an embodiment the aqueous composition further comprises a surfactant wherein the surfactant is selected from poloxamer surfactants (e.g., poloxamer 188), polysorbate surfactants (e.g., polysorbate 80 and/or polysorbate 20), octoxinal surfactants, polidocanol surfactants, polyoxyl stearate surfactants, polyoxyl castor oil surfactants, N-octyl-glucoside surfactants, macrogol 15 hydroxy stearate, and combinations thereof. In an embodiment, the surfactant is selected from poloxamer surfactants (e.g., poloxamer 188), polysorbate surfactants (e.g., polysorbate 80 and/or polysorbate 20), in particular polysorbate surfactants such as polysorbate 80. In an embodiment, the aqueous composition comprises a surfactant wherein the surfactant is present in an amount of at least 0.001% (w/v), at least 0.005% (w/v), at least 0.01% (w/v), and/or up to 0.5% (w/v). In an embodiment the surfactant is present in an amount less than 0.25% (w/v) or less than 0.1% (w/v). In another embodiment, the surfactant is present in an amount of 0.02% (w/v). According to specific embodiments, the surfactant is polysorbate 80 or poloxamer 188 present in the aqueous mixture in an amount between 0.005% and 0.5% (w/v), such as about 0.02% (w/v).

In a further embodiment, the aqueous composition (and thus the lyophilised pharmaceutical composition) further comprises a buffer. The pH is typically adjusted in view of the therapeutic components of the composition. Suitably, the pH of the aqueous composition is at least 6, at least 6.5, at least 7 or at least 7.5. Alternatively stated, the pH of the aqueous composition may be less than 10, less than 9.5, less than 9 or less than 8.5. In other embodiments, pH of the aqueous composition is between 6 and 10, between 7 and 9.5, between 7.5 and 9.5, or, about 8, for example 8±1 or 8±0.5. An appropriate buffer may be selected from Tris, succinate, borate, Tris-maleate, lysine, histidine, glycine, glycylglycine, citrate, carbonate, phosphate, or combinations thereof. In one embodiment, the aqueous composition further comprises a buffer and the buffer is Tris. In a particular embodiment, the aqueous composition further comprises a Tris buffer and wherein the pH of the aqueous composition is 8±0.5

In an embodiment, the buffer is present in the aqueous composition in an amount of at least 0.5 mM, at least 1 mM, at least 2 mM or at least 5 mM. In an embodiment, the buffer is present in the aqueous composition in an amount of less than 50 mM, less than 40 mM, or less than 30 mM. For example, the buffer may be present in the aqueous composition in an amount of 0.5 mm to 50 mM, 1 mM to 50 mM or 2 mM to 30 mM. In one embodiment, the buffer is present in the aqueous composition in an amount of 20±2 mM. According to specific embodiments, the buffer is Tris, present in the aqueous composition in an amount between 15 and 25 mM, optionally 20 mM.

In an embodiment, the aqueous composition (and thus the lyophilised pharmaceutical composition) also comprises histidine in an amount of up to or about 20 mM, such as at a concentration of about 10 mM. According to further embodiments, the composition also comprises bivalent metal ions, such as Mg²⁺, Ca²⁺, or Mg²⁺ or Ca²⁺ in the form of a salt, such as MgCI₂, CaCI₂ or MgSO₄. In one embodiment the bivalent metal ion is Mg²+. Typical amounts wherein the bivalent metal ions are present in the aqueous mixture are between 0.5 and 10 mM, such as 1 or 2 mM, or 1 mM in particular.

It will be acknowledged that a number of preparatory steps may be performed prior to step i) in the method of the fifth aspect. For example, prior to step i) at least one vessel may be sterilized.

In a preferred embodiment prior to step i) the aqueous composition is formulated and transferred into a vessel(s). In an embodiment the vessel(s) are pre-cooled prior to receiving the aqueous composition. It is preferable to formulate the aqueous composition prior to transferring the aqueous composition into the vessel(s) to ensure that a consistent and homogenous aqueous composition is present within each vessel. It will be understood that transferring the aqueous composition into a vessel(s) may also be referred to as “filling” at least one vessel(s) with the aqueous composition. In an embodiment transferring (or filling) at least one vessel(s) is performed with the aid of a filling line.

In an embodiment each vessel comprises one unit dose of the pharmaceutical composition (e.g., one unit dose of vaccine). In an embodiment, between 0.1 and 1.5 mL, 0.1 and 1 mL, 0.25 and 1 mL, 0.4 and 1 mL, 0.4 and 0.8 mL, 0.5 and 0.8 mL, 0.5 and 0.75 mL, 0.6 and 0.8 mL or 0.65 and 0.75 mL of said aqueous composition is transferred into a vessel(s). In an embodiment, less than 1.5 mL, less than 1.25 mL, less than 1 mL or less than 0.75 mL of said aqueous composition is transferred into a vessel(s). In an embodiment greater than 0.1 mL, greater than 0.25 mL, greater than 0.5 mL or greater than 0.6 mL of said aqueous composition is transferred into a vessel(s). In an embodiment, 0.7 mL±0.2 mL of said aqueous composition is transferred into a vessel(s). In an embodiment, 0.7 mL of said aqueous composition is transferred into a vessel(s).

It will be acknowledged that the method may comprise further steps following step iii). In an embodiment the method further comprises (i.e., following step iii) plugging the vessel, optionally wherein the vessel is plugged with a stopper. In an embodiment the stopper is siliconized and is configured to be positioned at least partially in the vessel. In an embodiment, following plugging the vessel, the vessel is capped to secure the stopper in respect of the vessel. In an embodiment, the method further comprises (i.e., following step iii) labelling of the vessel. In an embodiment, the vessel comprising the pharmaceutical composition is stored under conditions suitable for long-term stability.

The vessel can be large vessels, but more particularly the vessels are vials or other such small vessels. In an embodiment the vessel has a substantially cylindrical shape and at least comprises an inner circumferential wall. The vessel has a limited internal volume which is typically between 2 and 50 ml which is sufficient for packaging a ready-to-use quantity of composition to be administered to a subject e.g., a human. In an embodiment the interval volume of the vessel is 3 mL±0.5 mL. The vessel is at least partially made of a material which is translucent for electromagnetic radiation, in particular infrared, ultraviolet, and/or visible light. In an embodiment the vessel is plastic or glass. In an embodiment the vessel is glass. In an embodiment the vessel is a container that is configured to contain a ready-to-use quantity of the lyophilised pharmaceutical composition of the first aspect.

In an embodiment the vessel is of about 10 to 40 mm in diameter and carries at least one unit dose of drug or vaccine. In an embodiment the vessel carries at least one unit dose of vaccine. In an embodiment the vessel is a vial. In an embodiment the vial is a non-siliconized glass vial.

A plurality of said vessel(s) can be filled with the aqueous composition and loaded into an apparatus for carrying out the method of the fifth aspect. The total vessel load is a function of the apparatus design.

The method of the fifth aspect readily lends itself to incorporation in a continuous or semi-continuous freeze-drying process. In such a process the vessels are moved automatically through the various steps up to and including being subjected to the vacuum drying conditions. As such, in an embodiment the method is semi-continuous or continuous. In an embodiment the method is not conducted in batch-mode. A “continuous” or “semi-continuous method is a method in which the lyophilised pharmaceutical composition is produced substantially without interruption. Without interruption means that at no step is the process completely ceased and restarted at a later period of time. In the present context, the term continuous or semi-continuous both refer to the substantially uninterrupted transfer of constituents through the steps of the process. In other words, the movement of vessel(s) comprising the aqueous composition is such that the vessel(s) do not substantially remain in one location in the system, or at one step, for a significant or undesired period of time.

In an embodiment, the method is continuous wherein the method does not require any upstream steps to run to completion before downstream steps may commence. For example, a new batch of vessels can commence the spin-freezing stage (i.e., step i) and ii) in the method) whilst an earlier batch is being dried (i.e., step iii) in the method).

In an embodiment, the process is semi-continuous wherein the constituents (i.e., the vessels comprising the aqueous composition) transfer uninterrupted through all the steps in the method, but at least one step in the method must be completed before at least one downstream step may commence. In an embodiment, the process is semi-continuous wherein the constituents (i.e., the vessels comprising the aqueous composition) transfer uninterrupted through all the steps in the method, but during the process at least one upstream step must be run to completion before a downstream step can commence.

Step ii) refers to the stage wherein the aqueous composition is subjected to freezing conditions sufficient to freeze the aqueous composition. In an embodiment step ii) takes place in a freezing chamber.

Step iii) refers to the stage wherein the frozen composition is dried i.e. wherein the aqueous composition that was frozen during step ii) is dried. In an embodiment step iii) place in a drying chamber, optionally wherein the drying chamber comprises a cooling system attached thereto. The drying chamber is where the process of both sublimation (i.e. of ice crystals formed in the frozen composition) and desorption takes place. As such the drying chamber is configured to carry out both sublimation (primary drying) and desorption (secondary drying). As such the drying chamber is provided with a heating means, wherein said heating means facilitates sublimation and successive desorption. In an embodiment, the drying chamber is provided with at least one vacuum pump for applying pressure to the vessel(s), wherein said vessel(s) comprise the frozen composition.

In an embodiment, the drying chamber comprises a cooling system attached thereto wherein the cooling system utilizes dry ice to lower the temperature of the drying chamber walls. This results in decreased energy coming from the surroundings and thus a reduced/conservative primary drying temperature (i.e., the product temperature during the primary drying stage).

In an embodiment, the freezing chamber and the drying chamber are separated by an intermediate compartment. In an embodiment, the intermediate compartment is a load-lock. Such a load-lock is commonly formed by a revolving door via which the vessel is transported from one chamber to the other chamber.

In an embodiment the freezing chamber and the drying chamber are connected in succession. By means of a transporting means the vessel(s) can be guided along or through each chamber.

In an embodiment the transporting means is a robotic arm. In an embodiment a robotic arm transfers vessels from the freezing chamber to the drying chamber, optionally via the intermediate compartment.

With regards to the method of the fifth aspect, during step i) the surface area to volume ratio of the aqueous composition comprising nucleic acid (e.g., RNA) and lipid carrier particles is increased. In an embodiment, the surface area to volume ratio is increased compared to the surface area to volume ratio of the aqueous composition when it is present in its settled state in the bottom of the vessel.

In an embodiment, the surface area to volume ratio is increased compared to the surface area to volume ratio of the aqueous composition prior to step i). As used herein the term “surface area” is the total area of the exposed surface of the aqueous composition. In the context of surface area to volume ratio, the term “volume” refers to the volume (e.g., in mL) of aqueous composition. Increasing the surface area to volume ratio of the aqueous composition provides a greater surface area for freeze-drying, without requiring an increased volume of said aqueous composition. In an embodiment, the surface area to volume ratio is increased by spreading the composition out over the inner surface of a circumferential wall of the vessel.

In an embodiment, during step i) the surface area to volume ratio is increased by greater than 1.5 times, greater than 2 times, greater than 3 times or greater than 4 times compared to the surface to area volume ratio prior to step i). In an embodiment during step i) the surface area to volume ratio is increased by between 1× and 10×, between 2× and 8×, between 3× and 7× or between 3× and 5× compared to the surface area to volume ratio prior to step i). In an embodiment during step i) the surface area to volume ratio is increased by around 4×±1× compared to the surface area to volume ratio prior to step i). The surface area to volume ratio prior to step i) reflects the surface area to volume ratio of the aqueous composition when present in the bottom of the vessel.

In an embodiment, increasing the surface area to volume ratio of the aqueous composition is achieved by spraying the aqueous composition comprising nucleic acid and lipid carrier particles to form droplets. In an embodiment the droplets comprise an average diameter ranging from about 20 μm to about 100 μm. In an embodiment, spraying of the aqueous composition can be conducted using common moderate pressure spraying, supercritical spraying, high pressure spraying, atomization, and/or the like. In an embodiment the droplets are then frozen in a fluid of cold liquid or gas (e.g., a gas of between −5° C. and −196° C.). In an embodiment the frozen droplets are dried to form a particulate.

However, in an embodiment, increasing the surface area to volume ratio of the aqueous composition is achieved by rotating a vessel containing the aqueous composition at a speed not less than that required to form and maintain the aqueous composition in a layer of substantially uniform thickness against an inner surface of a circumferential wall of the vessel. In an embodiment, said rotation is at a speed and for a period of time not less than that required to form and maintain the aqueous composition in a layer of substantially uniform thickness against an inner surface of a circumferential wall of the vessel. It will be understood by the skilled person that the speed of rotation and/or time period required to form and maintain the aqueous composition in a layer of substantially uniform thickness against the inner surface of the circumferential wall of the vessel may differ depending on a number of factors, for example the viscosity of the aqueous composition, the volume of aqueous composition in the vessel and the dimensions and/or shape of the vessel. If the speed of rotation is too low the aqueous composition will not be held in said layer against the inner surface of the circumferential wall of the vessel. In an embodiment, increasing the surface area to volume ratio of the aqueous composition results in an even distribution of said aqueous composition around the inner surface of the circumferential wall of the vessel.

In an embodiment, the vessel is axially rotated. Said axial rotation results in the formation of a relatively thin layer of aqueous composition on the inner surface of the circumferential wall of the vessel due to centrifugal forces. In an embodiment the vessel containing aqueous composition is rotated about its longitudinal axis. In an embodiment, the vessel containing aqueous composition is rotated about its longitudinal axis whilst supported in a substantially horizontal orientation. Accordingly, the vessel containing the aqueous composition is tilted from a substantially vertical orientation to a substantially horizontal orientation during step i).

In an embodiment, the vessel containing the aqueous composition is rotated (e.g., axially rotated) at a speed of between 250 and 4000 revolutions per minute (rpm), between 500 and 4000 rpm, between 1000 and 4000 rpm, between 1500 and 3750 rpm, between 2000 and 4000, between 2000 and 3750 rpm, between 2000 and 3500 rpm, between 2500 and 4000 rpm, between 2500 and 3500 rpm, between 3000 and 4000 rpm, between 3000 and 3500 rpm or between 3100 and 3300 rpm. In an embodiment, the vessel containing the aqueous composition is rotated at a speed of between 2500 and 4000 rpm, optionally between 3000 and 3500 rpm, optionally at 3200 rpm. In an embodiment the vessel containing the aqueous composition is rotated at a speed of 3200 rpm±100 rpm. In an embodiment the vessel containing the aqueous composition is rotated at a speed of 3200 rpm.

Once the vessel containing the aqueous composition has been rotated at a speed not less than that required to form and maintain the aqueous composition in a layer of substantially uniform thickness against the inner surface of the circumferential wall of the vessel, the temperature of the vessel (and thus the temperate of the aqueous composition within the vessel) is reduced to below 0° C.

In an embodiment during step ii) a predefined cooling temperature profile or scheme is used. In an embodiment freezing of the aqueous composition may be effectuated by surrounding the vessel comprising the aqueous composition by a cooling gas, in particular an inert gas having a controlled temperature. For example, the temperature and/or flow speed of said cooling gas may be adjusted dependent on the actual temperatures detected and the temperature profile to be applied.

In an embodiment during step ii) freezing is achieved by contacting the vessel containing the aqueous composition with an inert cooling gas. In an embodiment, the inert cooling gas is at a temperature of −5 to −160° C. Freezing of the composition during step ii) is thus realized by using at least one inert cooling gas, such as nitrogen, wherein said cooling gas surrounds the vessel(s) to cool down the composition. In an embodiment the cold insert gas is nitrogen gas. During freezing (step ii) the temperature of the vessel and surrounding atmosphere is reduced such that the aqueous composition in the vial becomes frozen immobile or solid. In an embodiment, during step ii) the aqueous composition becomes a frozen composition. Once the aqueous composition is frozen, the remainder of the freezing cycle may then be accomplished without further spinning of the vessel.

In an embodiment, during step ii) the aqueous composition is frozen at a rate of between 1 and 200° C./min, 5 and 175° C./min, 10 and 150° C./min, 20 and 150° C./min, 50 and 125° C./min or 75 and 105° C./min. In an embodiment the aqueous composition is frozen at a rate of between 5° and 150° C./min. In an embodiment the aqueous composition is frozen at a rate of between 75 and 125° C./min, optionally 100° C./min. In an embodiment, the aqueous composition is frozen at a rate of 100° C./min±5° C./min. In an embodiment, the aqueous composition is frozen at a rate of 100° C./min. In an embodiment, the aqueous composition is frozen at a rate of greater than 5° C./min, greater than 20° C./min, greater than 50° C./min, greater than 75° C./min or greater than 90° C./min. In an embodiment, the aqueous composition is frozen at a rate of less than 200° C./min, less than 175° C./min, less than 150° C./min or less than 125° C./min.

In an embodiment, the aqueous composition is frozen in step ii) until a final vessel temperature below the critical temperature of the aqueous composition is reached. The critical temperature is the temperature at, or below which, an aqueous composition is solidified. In an embodiment the critical temperature is the glass transition temperature (Tg′). In an embodiment, the critical temperature is the collapse temperature (Tcol). Since the Tg′ is lower than the Tcol, in an embodiment, the aqueous composition is frozen in step ii) until a final vessel temperature below the Tg′ of the aqueous composition is reached. The skilled person is familiar with techniques to assess and monitor a composition's Tg′ for example using differential scanning calorimetry.

In an embodiment the critical temperature is between −10° C. and −100° C., between −15° C. and −80° C., between −20° C. and −60° C., between −25° C. and −50° C. or between −25° C. and −40° C. In an embodiment the critical temperature is between −29° C. and −35° C. In an embodiment the critical temperature is around −30° C.

In an embodiment, the aqueous composition is frozen in step ii) until a final vessel temperature of between −4 and −200° C., between −10 and −175° C., between −30 and −150° C., between −30 and −100° C., between −30 and −80° C., between −40 and −100° C., between −60 and −100° C. or between −60 and −80° C. is reached. In an embodiment, the aqueous composition is frozen until a final vessel temperature of −70° C.±10° C. or −70° C.±5° C. is reached. In an embodiment, the aqueous composition is frozen until a final vessel temperature of −70° C. is reached.

In an embodiment the aqueous composition is rapidly snap frozen in step ii). Following step i) the surface area to volume ratio of the aqueous composition comprising nucleic acid and lipid carrier particles is increased. Said increase in surface area to volume ratio results in the composition forming a thin (e.g., approximately 1-4 mm) layer of the substantially uniform thickness, wherein said think layer enables the snap freezing of the composition. In an embodiment, the aqueous composition is rapidly snap frozen in step ii) for a duration of between 10 and 300 seconds, 10 and 240 seconds, 20 and 180 seconds, 20 and 140 seconds, 20 and 120 seconds, 20 and 100 seconds or 30 and 90 seconds. In an embodiment, the aqueous composition is frozen for a duration of 30 to 60 seconds. The freezing cycle may however last longer than merely the time taken to rapidly freeze the composition, i.e., once snap frozen, the vessel may be maintained at freezing temperatures for an extended duration of time.

In an embodiment, there is provided a method of producing the lyophilised pharmaceutical compositions provided herein, said method comprising, following step ii), a step iii) wherein the frozen composition is dried. In an embodiment, the method provides an initial freezing step, followed by a primary drying step, followed by a secondary drying step. In an embodiment the primary drying step is referred to as a sublimation step and the secondary drying step is called the desorption step. In an embodiment there is provided a method for producing the pharmaceutical composition of the first aspect said method comprising
i) increasing the surface area to volume ratio of an aqueous composition comprising nucleic acid and lipid carrier particles
ii) subjecting the aqueous composition to freezing conditions sufficient to freeze the aqueous composition
iii) drying the frozen composition wherein said drying comprises both a primary and a second drying step.

In an embodiment, during step iii) the frozen composition is subjected to a primary drying step, and after the primary drying step, is subjected to secondary drying step, both primary and secondary drying steps taking place within a drying chamber. In an embodiment, the primary drying step is immediately followed by the secondary drying step.

The primary drying step comprises drying the frozen composition to sublime at least a portion of the ice crystals formed within the frozen composition by substantially heating the frozen composition, said frozen composition being contained within a vessel(s), said vessel(s) being within the drying chamber. During the sublimation process water vapor will be generated which leaves the surface of the frozen composition. Once a substantial part of the ice crystals has been removed a porous structure of the composition remains (referred to herein as the cake).

In an embodiment, the primary drying step comprises adjusting the temperature of the frozen composition to a primary drying temperature under a vacuum. As such, as used herein “primary drying temperature” relates to the product temperature or the temperature of the composition as during the primary drying step, as measured using a sensor, for example an infrared camera. The primary drying temperature can be achieved by modulating either the temperature and/or the pressure of the drying chamber.

In an embodiment, the primary drying temperature is both below the collapse temperature (Tcol) and the Tg′ of the frozen composition. If the primary drying temperature exceeds the Tcol, the cake collapses and loses its structure. As such, in an embodiment the primary drying temperature is below the Tcol.

In an embodiment, the Tg′ is determined by differential scanning colorimetry. In an exemplified embodiment the Tg′ is 33° C.±1° C.

In an exemplified embodiment the Tcol is determined using freeze-drying microscopy and is −31° C.±0.5° C.

In an embodiment, the primary drying temperature is below the collapse temperature but is at or above the Tg′ of the composition. In an embodiment, the primary drying temperature is between the collapse temperature and the Tg′ of the composition. It has been found that drying the frozen composition at a temperature below the collapse temperature but above the Tg′ provides an opportunity to sublime a major portion of the ice crystals present within the frozen composition in the shortest period of time. If the primary drying temperature is below the Tg′ the primary drying step takes longer than if the primary drying temperature is at or slightly above the Tg′ (but below the Tcol) assuming the vacuum is maintained at the same pressure. In an embodiment, the primary drying temperature is less than 20%, less than 10% or less than 5% lower than the collapse temperature of the frozen composition. When drying the frozen composition at temperatures close to the Tcol it is recommended to monitor for minor extents of collapse e.g. using scanning electron microscopy (e.g., as described in Depaz et al. (2016) J Pharm Sci, 105(1):40-9).

In an embodiment, the primary drying temperature is between −20° C. and −60° C., between −25° C. and −45° C. or between −30° C. and −40° C. In an embodiment the primary drying temperature is between −28° C. and −35° C. In an embodiment, the primary drying temperature is −29° C.±0.5° C., −30° C.±0.5° C., −31° C.±0.5° C., −32° C.±0.5° C. or −33° C.±0.5° C., −34° C.±0.5° C., −35° C.±0.5° C., −36° C.±0.5° C., −37° C.±0.5° C., −38° C.±0.5° C. or −39° C.±0.5° C., −40° C.±0.5° C., −41° C.±0.5° C., or −42° C.±0.5° C.

In an embodiment, the drying chamber is pre-cooled to a temperature at least below the collapse temperature of the composition. In an embodiment, the primary drying temperature is achieved from surrounding heat energy only. Achievement of the primary drying temperature from surrounding heat energy means that the primary drying temperature is achieved without the requirement for a specific heat source e.g., without the need for a radiator.

In an embodiment the vacuum is at a pressure below the triple point of water. At pressures below the triple point, and when thermal energy is supplied, solid ice is converted directly into water vapour, resulting in sublimation of ice during the primary drying step. The vacuum is typically realised using a vacuum pump. In an embodiment, the vacuum is at a pressure of between 10 and 150 μbar, 20 and 125 μbar, 25 and 110 μbar or 30 and 100 μbar. In an embodiment the vacuum is at a pressure of 60±2 μbar, 65 μbar, 70±2 bar, 75±2 bar, 80±2 bar, 85±2 bar, 90±2 bar, 95±2 bar or 100±2 μbar. In an embodiment, the vacuum is at a pressure of 80 μbar. The pressure of the vacuum is expressed in terms of pbar e.g., pbar Pirani pressure. It will however be understood that the equivalent pressures expressed in other units is within the scope of present disclosure. For example, equivalent pressures expressed in psi, mTorr, pascal etc. fall within the scope of the present disclosure. It is understood by the skilled person that vacuums of low pressure (e.g., 10 μbar) result in a shorter primary drying speed compared to vacuums of higher pressure (e.g., 100 μbar). It is also understood that vacuums of higher pressure increase the primary drying temperature i.e., the product temperature. Caution is required to ensure that increasing the pressure does not result in a primary drying temperature above the collapse temperature of the composition.

Increasing the surface area to volume ratio during step i) of the method of the fifth aspect further permits primary drying to occur rapidly. This may be because a greater surface area exists for the water molecules to leave the frozen composition compared to situations where the surface area to volume ratio is not increased. In an embodiment, the primary drying step lasts for a period of time sufficient to sublime a major portion or substantially all of the ice crystals within the frozen composition. In an embodiment primary drying step takes between 25 mins and 200 mins e.g., between 20 mins and 180 mins, between 30 mins and 160 mins or between 60 mins and 100 mins. In an embodiment, the primary drying step is 90 mins±30 mins, 90 mins±20 mins or 90 mins±10 mins. In an embodiment the primary drying step is approximately 90 mins. In an embodiment, the primary drying step is less than 30 hours, less than hours, less than 20 hours, less than 15 hours, less than 12 hours, less than 10 hours, less than 8 hours, less than 6 hours, less than 5 hours, less than 4 hours, less than 3 hours, less than 2.5 hours or less than 2 hours.

In an embodiment water vapour escaping from the frozen composition is removed from the vessel(s) by using at least one separate (cryogenic) ice condenser which makes the water vapour (re)sublime to ice crystals and/or condense to liquid water which precipitate on and/or in the ice condenser.

In addition to the free ice that is sublimed during the primary drying step there commonly remains a substantial amount of water molecules that are (ionically) bound (adsorbed) to the composition. At the end of the primary drying step, the composition will typically have 5 to 15% moisture content. This remaining water fraction is removed by a secondary drying step, also referred to as desorption step. Thus, in an embodiment, the secondary drying step comprises adjusting the temperature of the composition to a secondary drying temperature under vacuum. As used herein the term “secondary drying temperature” relates to the product temperature or the temperature of the composition during the secondary drying step, as measured using a sensor, for example an infrared camera. In an embodiment the secondary drying temperature is the temperature of the cake during the secondary drying step.

Since substantially all of the free ice has been removed in primary drying, the composition temperature can now be increased considerably without fear of melting or collapse. In an embodiment, the secondary drying temperature is above 0° C. In an embodiment the secondary drying temperature is between 5° C. and 50° C., 10° C. and 45° C. or 20 and 40° C. In an embodiment, the secondary drying temperature is 32° C. to 38° C., optionally 35° C. In an embodiment, the secondary drying temperature is 32° C.±0.5° C., 33° C.±0.5° C., 34° C.±0.5° C., 35° C.±0.5° C., 36° C.±0.5° C., 37° C.±0.5° C., or 38° C.±0.5° C.

Secondary drying actually starts during the primary drying phase (sublimation), but as described herein, the secondary drying step is considered to start at the point that the secondary drying temperature is raised to at least above 0° C. At elevated temperatures (typically in the 25° C. to 50° C. range), desorption proceeds much more quickly. Secondary drying rates are dependent on the composition temperature. In an embodiment, the temperature increase from primary to secondary drying is controlled at a slow ramp rate for example to avoid cake collapse. In an embodiment the ramp rate is 0.1° C./min to 5° C./min. In an embodiment the ramp rate is between 0.1° C./min and 5° C./min, 0.5° C./min and 4° C./min or 1° C./min to 3.5° C./min. In an embodiment the ramp rate is 3° C./min±1° C./min. In an embodiment the ramp rate is 3° C./min.

During the secondary drying step, the vacuum may be maintained at substantially the same level (e.g., ±5%, 10%, 15%, 20% or 25%) to that used during the primary drying step; because, unlike during the sublimations step, lower vacuum levels will not improve secondary drying times. In an embodiment, during secondary drying the vacuum is at a pressure of between 10 and 150 μbar, 20 and 125 μbar, 25 and 110 μbar or 30 and 100 μbar. In an embodiment the vacuum is at a pressure of 60±2 μbar, 65 μbar, 70±2 bar, 75±2 bar, 80±2 bar, 85±2 bar, 90±2 bar, 95±2 bar or 100±2 μbar. In an embodiment, the vacuum is at a pressure of 80 μbar.

During the secondary drying step use is made of at least one heat conducting means and/or at least one heat reflecting means to substantially homogeneously heat the drying chamber (and thus the vessels comprising the composition). During secondary drying at least one heat source is used, wherein the at least one heat source is configured to generate electromagnetic radiation, in particular infrared radiation (wavelength 750 nm to 1 mm) and/or microwaves (wavelength 1 mm to 1 meter). In an embodiment the secondary drying temperature is achieved by heat energy provided by microwaves, a heating element(s) or an infrared radiator(s). In a preferred embodiment, the heat energy is provided by an infrared radiator(s).

Secondary drying is continued until the composition has acceptable moisture content for long term storage. Accordingly, in an embodiment the secondary drying step is effected for a period of time sufficient to result in the lyophilised pharmaceutical composition having a moisture content of between 0.25% and 3%. Depending on the application, moisture content in fully dried compositions is typically between 0.25% and 3% e.g., between 0.25 and 2.5% (w/w), between 0.5 and 2% (w/w), between 0.5 and 1.25% (w/w), or between 0.5 and 1% (w/w). In most cases, the more dry the composition, the longer its shelf life will be. However, certain complex biological compositions may actually become too dry for optimum storage results and the secondary drying process (the desorption step) should be controlled accordingly. Residual moisture content is determined by techniques known to the skilled person such as the Karl Fisher method. In an embodiment the secondary drying step takes between 30 mins and 180 mins, between 30 mins and 120 mins or between 60 mins and 100 mins. In an embodiment primary drying step takes between 30 mins and 180 mins, between 30 mins and 160 mins or between 60 mins and 100 mins. In an embodiment, the secondary drying step is less than 30 hours, less than 25 hours, less than 20 hours, less than 15 hours, less than 12 hours, less than 10 hours, less than 8 hours, less than 6 hours, less than 5 hours, less than 4 hours, less than 3 hours, or less than 2 hours.

In an embodiment, step iii) takes less than 24 hours, less than 22 hours, less than 20 hours, less than 18 hours, less than 16 hours, less than 14 hours, less than 12 hours, less than 10 hours, less than 8 hours, less than 6 hours or less than 4 hours. In an embodiment, the primary and secondary drying steps combined take less than 24 hours, less than 22 hours, less than 20 hours, less than 18 hours, less than 16 hours, less than 14 hours, less than 12 hours, less than 10 hours, less than 8 hours, less than 6 hours or less than 4 hours.

In an embodiment during step iii) the temperature of the composition and/or the vessel(s) comprising the composition is measured using a sensor. In an embodiment the sensor is an infrared camera. In an embodiment, the temperature of the composition and/or the vessel(s) is further monitored during step ii).

In a sixth aspect, there is provided a freeze-dried composition obtained from the method of the fifth aspect.

The pharmaceutical composition, vaccine or freeze-dried composition of the present disclosure is for use in medicine.

As such, in a seventh aspect there is provided the use of the pharmaceutical composition of the first aspect, the vaccine of the second aspect or the kit of the fourth aspect in the manufacture of a medicament for treating a subject in need thereof. Furthermore, in an eighth aspect there is provided the use of the pharmaceutical composition of the first aspect, the vaccine of the second aspect or the kit of the fourth aspect in the manufacture of a medicament for prophylaxis in a subject in need thereof.

In a ninth aspect there is provided a method for eliciting an immune response in a subject in need thereof comprising administering the pharmaceutical composition of the first aspect or the vaccine of the second aspect to the subject, optionally wherein the subject is a human subject. It will be understood that said administration of the pharmaceutical composition of the first aspect or the vaccine of the second aspect is following reconstitution. In an embodiment, the pharmaceutical composition or vaccine is for eliciting an immune response in vivo against an immunogen of interest. In an embodiment, the immune response is protective and involves antibodies and/or cell-mediated immunity. The method may raise a booster response. By raising an immune response, the subject can be protected against various diseases and/or infections e.g., against bacterial and/or viral diseases as discussed above. RNA-containing compositions are immunogenic and are more preferably vaccine compositions. Vaccines according to the invention may either be prophylactic (i.e., to prevent infection) or therapeutic (i.e. to treat infection), but will typically be prophylactic.

In a tenth aspect there is provided the pharmaceutical composition of the first aspect or the vaccine of the second aspect for use in medicine.

In an eleventh aspect there is provided the pharmaceutical composition of the first aspect or the vaccine of the second aspect for use in the treatment or prevention of disease in a subject, optionally wherein the subject is a human subject. In an embodiment, the subject is a mammal, such as a human or a large veterinary mammal. In an embodiment, the subject is a human. The pharmaceutical composition and vaccines prepared according to the disclosure may be used to treat both children and adults. Thus, a human patient may be less than 1 year old, less than 5 years old, 1-5 years old, 5-15 years old, 15-55 years old, or older than 55 years old.

Pharmaceutical compositions and vaccines of the invention will generally be administered directly to a patient. Direct delivery may be accomplished by any method of administration known to the skilled person such as via parenteral injection (e.g., subcutaneously, intraperitoneally, intravenously, intramuscularly or to the interstitial space of a tissue) or mucosally, such as by rectal, oral (e.g., tablet, spray), vaginal, topical, transdermal or transcutaneous, intranasal, ocular, pulmonary or other mucosal administration. Injection may be via a needle (e.g., a hypodermic needle), but needle-free injection may alternatively be used. A typical intramuscular dose is 0.5 ml. The pharmaceutical compositions and vaccines disclosed herein may be used to elicit systemic and/or mucosal immunity.

Dosage can be by a single dose schedule or a multiple dose schedule. Multiple doses may be used in a primary immunization schedule and/or in a booster immunization schedule. In a multiple dose schedule the various doses may be given by the same or different routes e.g., a parenteral prime and mucosal boost, a mucosal prime and parenteral boost, etc. Multiple doses will typically be administered at least 1 week apart (e.g., about 2 weeks, about 3 weeks, about 4 weeks, about 6 weeks, about 8 weeks, about 10 weeks, about 12 weeks, about 16 weeks, etc.). In an alternative embodiment, two primary doses are administered about two months apart, e.g., about 7, 8 or 9 weeks apart, followed by one or more booster doses about 6 months to 1 year after the second primary dose, e.g., about 6, 8, 10 or 12 months after the second primary dose. In a further embodiment, three primary doses are administered about two months apart, e.g., about 7, 8 or 9 weeks apart, followed by one or more booster doses about 6 months to 1 year after the third primary dose, e.g., about 6, 8, 10, or 12 months after the third primary dose.

In some embodiments, the subject is human. In some embodiments, the subject is a mammal, such as a human or a large veterinary mammal (e.g. horses, cattle, deer, goats, pigs). Where the formulation or recombinant RNA is for eliciting a protective immune response, the subject is preferably a human, such as a child (e.g. a toddler or infant), a teenager, and the recombinant RNA or the formulation comprising the recombinant RNA is formulated as a vaccine. Where the composition or recombinant RNA is used as a treatment or for therapeutic use, the human is preferably a teenager or an adult. A vaccine intended for children may also be administered to adults, with the provisio that the amount of recombinant RNA or formulation comprising the recombinant RNA may be scaled up to provide an unit dose consistent with the state of the immune system of the subject (i.e. the elderly having more difficulty eliciting certain immune responses) and the average body weight of a subject of that age or the actual body weight of the subject.

In some embodiments, the recombinant RNA or formulation comprising the recombinant RNA is administered to the subject intramuscularly, intradermally, subcutaneously, transcutaneously, topically, intraperitoneally, intrathecally, pulmonarily (i.e. inhaled), intracerebroventricularly, intravenously, intra-arterially, onto a mucosa (i.e. vaginally), buccally, sublingually, intranasally, optically, to the cornea, or into the eyeball.

In some embodiments, a method for treating cancer in a subject is provided, the subject having a tumor of the cancer, the method comprising administering to the subject the formulation comprising the recombinant RNA or the recombinant RNA or an unit dose thereof. In some embodiments, the recombinant RNA comprises a sequence that encodes a heterologous polypeptide comprising a polyepitopic peptide comprising two or more immunogenic neo-epitopes and a linker, the linker linking the two or more immunogenic neo-epitopes, the immunogenic neo-epitopes being from a first sample comprising cells from the tumor from the subject, each neo-epitope being: (a) encoded in mRNA in the first sample, (b) occurring in a protein-coding region therein, (c) being predicted to bind to a major histocompatibility complex, and (d) being capable of introducing a difference in the amino acid sequence of the neo-epitope when compared to a reference amino acid sequence or genetic sequence predicted to encode the reference amino acid sequence obtained from a second sample from a non-cancerous cell from the subject. In some embodiments, the recombinant RNA is produced from a method comprising: obtaining a first nucleic acid sequence from the first sample, obtaining a second nucleic acid sequence from the second sample, comparing the first nucleic acid sequence to the second nucleic acid sequence thereby obtaining at least two somatic mutations present in the tumor cells, identifying from the at least two somatic mutations (a)-(d), and producing the recombinant RNA.

In one aspect, a method for treating cancer in a subject is provided, the method comprising administering to the subject the recombinant RNA, a formulation comprising the recombinant RNA, or an unit dose thereof, wherein the recombinant RNA comprises a sequence encoding a heterologous polypeptide comprising IL-12sc, IL-15sushi, IFNα, or GM-CSF. In some embodiments, the method further comprises administering an anti-PD-1/PD-L1 checkpoint inhibitor. In some embodiments, the cancer is melanoma, head and neck squamous cell cancer (HNSCC), cutaneous squamous cell carcinoma (CSCC), or advanced anti-PD-1/PD-L1 naïve cancers thereof.

In some embodiments, a method for treating cancer in a subject is provided, the method comprising administering to the subject the recombinant RNA, a formulation comprising the recombinant RNA, or an unit dose thereof. In some embodiments, the recombinant RNA comprises a sequence encoding a heterologous polypeptide comprising autogene, cevumeran, or atezolizumag. In some embodiments, the cancer is melanoma, head and neck squamous cell cancer (HNSCC), cutaneous squamous cell carcinoma (CSCC), non-small cell lung cancer (NSCLC), or advanced anti-PD-1/PD-L1 naïve cancers thereof.

In some embodiments, the unit dose comprises or is at least: 0.1 μg, 1 μg, 2 μg, 3 μg, 4 μg, 5 μg, 6 μg, 7 μg, 8 μg, 9 μg, 10 μg, 11 μg, 12 μg 13 μg, 14 μg, 15 μg, 16 μg, 17 μg, 18 μg, 19 μg, 20 μg, 21 μg, 22 μg, 23 μg, 24 μg, 25 μg, 26 μg, 27 μg, 28 μg, 29 μg, 30 μg, 31 μg, 32 μg, 33 μg, 34 μg, 35 μg, 36 μg, 37 μg 38 μg, 39 μg, 40 μg, 41 μg, 42 μg, 43 μg, 44 μg, 45 μg, 46 μg, 47 μg 48 μg, 49 μg, 50 μg, 51 μg, 52 μg 53 μg, 54 μg, 55 μg, 56 μg, 57 μg, 58 μg, 59 μg, 60 μg, 61 μg, 62 μg, 63 μg, 64 μg, 65 μg, 66 μg, 67 μg, 68 μg, 69 μg, 70 μg, 71 μg, 72 μg, 73 μg, 74 μg, 75 μg, 76 μg, 77 μg, 78 μg, 79 μg, 80 μg, 81 μg, 82 μg 83 μg, 84 μg, 85 μg, 86 μg, 87 μg, 88 μg, 89 μg, 90 μg, 91 μg, 92 μg 93 μg, 94 μg, 95 μg, 96 μg, 97 μg, 98 μg, 99 μg, 100 μg, 101 μg, 102 μg, 103 μg, 104 μg, 105 μg, 106 μg, 107 μg, 108 μg, 109 μg, 110 μg, 111 μg, 112 μg 113 μg, 114 μg, 115 μg, 116 μg, 117 μg 118 μg, 119 μg, or 120 μg of recombinant RNA molecules. In some embodiments, the unit dose comprises or is no more than: 120 μg, 119 μg, 118 μg, 117 μg, 116 μg, 115 μg, 114 μg, 113 μg, 112 μg, 111 μg, 110 μg, 109 μg, 108 μg, 107 μg 106 μg, 105 μg, 104 μg, 103 μg, 102 μg, 101 μg, 100 μg, 99 μg, 98 μg, 97 μg, 96 μg, 95 μg, 94 μg, 93 μg, 92 μg, 91 μg, 90 μg, 89 μg, 88 μg, 87 μg, 86 μg, 85 μg, 84 μg, 83 μg, 82μ, 81 μg, 80 μg, 79 μg, 78 μg, 77 μg, 76 μg, 75 μg, 74 μg, 73 μg, 72 μg, 71 μg, 70 μg, 69 μg, 68 μg, 67 μg, 66 μg, 65 μg, 64 μg, 63 μg, 62 μg, 61 μg, 60 μg, 59 μg, 58 μg, 57 μg 56 μg, 55 μg, 54 μg, 53 μg, 52 μg 51 μg, 50 μg, 49 μg, 48 μg, 47 μg, 46 μg, 45 μg, 44 μg, 43 μg, 42 μg, 41 μg, 40 μg, 39 μg, 38 μg, 37 μg, 36 μg, 35 μg, 34 μg, 33 μg, 32 μg, 31 μg, 30 μg, 29 μg, 28 μg, 27 μg, 26 μg, 25 μg, 24 μg, 23 μg, 22 μg 21 μg, 20 μg, 19 μg, 18 μg, 17 μg 16 μg, or 15 μg of recombinant RNA molecules. Taking the above-noted embodiments into account, it is contemplated and supported that any of the above-noted “g” of “at least” and any of the above-noted “g” of “no more than” may be combined to provide an enclosed range (i.e. the unit dose comprises or is from 25 μg to 75 μg). In some embodiments, the unit dose comprises or is from 1 μg to: 2 μg, 3 μg, 4 μg, 5 μg, 6 μg, 7 μg, 8 μg, 9 μg, 10 μg, 11 μg, 12 μg, 13 μg, 14 μg, 15 μg, 16 μg, 17 μg, 18 μg, 19 μg, 20 μg, 21 μg, 22 μg, 23 μg, 24 μg, 25 μg, 26 μg, 27 μg, 28 μg, 29 μg, 30 μg, 31 μg, 32 μg, 33 μg, 34 μg, 35 μg, 36 μg, 37 μg, 38 μg, 39 μg, 40 μg, 41 μg, 42 μg, 43 μg, 44 μg, 45 μg, 46 μg, 47 μg, 48 μg, 49 μg, 50 μg, 51 μg, 52 μg, 53 μg, 54 μg, 55 μg, 56 μg, 57 μg, 58 μg, 59 μg, 60 μg, 61 μg, 62 μg 63 μg, 64 μg, 65 μg, 66 μg, 67 μg, 68 μg, 69 μg, 70 μg, 71 μg, 72 μg, 73 μg, 74 μg, 75 μg, 76 μg, 77 μg, 78 μg, 79 μg, 80 μg, 81 μg, 82 μg, 83 μg, 84 μg, 85 μg, 86 μg, 87 μg, 88 μg, 89 μg, 90 μg, 91 μg, 92 μg, 93 μg, 94 μg, 95 μg, 96 μg, 97 μg, 98 μg, 99 μg, 100 μg, 101 μg, 102 μg, 103 μg, 104 μg, 105 μg, 106 μg, 107 μg, 108 μg, 109 μg, 110 μg, 111 μg, 112 μg 113 μg, 114 μg, 115 μg, 116 μg, 117 μg 118 μg, 119 μg, or 120 μg; from 3 μg to: 4 μg, 5 μg, 6 μg, 7 μg, 8 μg, 9 μg, 10 μg, 11 μg, 12 μg, 13 μg, 14 μg, 15 μg, 16 μg, 17 μg, 18 μg, 19 μg, 20 μg, 21 μg, 22 μg, 23 μg, 24 μg, 25 μg, 26 μg, 27 μg, 28 μg, 29 μg, 30 μg, 31 μg, 32 μg, 33 μg, 34 μg, 35 μg, 36 μg, 37 μg, 38 μg, 39 μg, 40 μg, 41 μg, 42 μg, 43 μg, 44 μg, 45 μg, 46 μg, 47 μg 48 μg, 49 μg, 50 μg, 51 μg, 52 μg, 53 μg, 54 μg, 55 μg, 56 μg, 57 μg 58 μg, 59 μg, 60 μg, 61 μg, 62 μg, 63 μg, 64 μg, 65 μg, 66 μg, 67 μg 68 μg, 69 μg, 70 μg, 71 μg, 72 μg, 73 μg, 74 μg, 75 μg, 76 μg, 77 μg, 78 μg, 79 μg, 80 μg, 81 μg, 82 μg, 83 μg, 84 μg, 85 μg, 86 μg, 87 μg, 88 μg, 89 μg, 90 μg, 91 μg, 92 μg 93 μg, 94 μg, 95 μg, 96 μg, 97 μg, 98 μg, 99 μg, 100 μg, 101 μg, 102 μg, 103 μg, 104 μg, 105 μg, 106 μg, 107 μg, 108 μg, 109 μg, 110 μg, 111 μg, 112 μg, 113 μg, 114 μg, 115 μg, 116 μg, 117 μg, 118 μg, 119 μg, or 120 μg; from 5 μg to: 6 μg, 7 μg, 8 μg, 9 μg, 10 μg, 11 μg, 12 μg, 13 μg, 14 μg, 15 μg, 16 μg, 17 μg, 18 μg, 19 μg, 20 μg, 21 μg, 22 μg, 23 μg, 24 μg, 25 μg, 26 μg, 27 μg 28 μg, 29 μg, 30 μg, 31 μg, 32 μg, 33 μg, 34 μg, 35 μg, 36 μg, 37 μg 38 μg, 39 μg, 40 μg, 41 μg, 42 μg, 43 μg, 44 μg, 45 μg, 46 μg, 47 μg, 48 μg, 49 μg, 50 μg, 51 μg, 52 μg, 53 μg, 54 μg, 55 μg, 56 μg, 57 μg 58 μg, 59 μg, 60 μg, 61 μg, 62 μg 63 μg, 64 μg, 65 μg, 66 μg, 67 μg 68 μg, 69 μg, 70 μg, 71 μg, 72 μg 73 μg, 74 μg, 75 μg, 76 μg, 77 μg, 78 μg, 79 μg, 80 μg, 81 μg, 82 μg, 83 μg, 84 μg, 85 μg, 86 μg, 87 μg, 88 μg, 89 μg, 90 μg, 91 μg, 92 μg, 93 μg, 94 μg, 95 μg, 96 μg, 97 μg, 98 μg, 99 μg, 100 μg, 101 μg, 102 μg, 103 μg, 104 μg, 105 μg, 106 μg, 107 μg, 108 μg, 109 μg, 110 μg, 111 μg, 112 μg, 113 μg, 114 μg, 115 μg, 116 μg, 117 μg, 118 μg, 119 μg, or 120 μg; from 7 μg to: 7 μg 8 μg, 9 μg, 10 μg, 11 μg, 12 μg 13 μg, 14 μg, 15 μg, 16 μg, 17 μg, 18 μg, 19 μg, 20 μg, 21 μg, 22 μg, 23 μg, 24 μg, 25 μg, 26 μg, 27 μg, 28 μg, 29 μg, 30 μg, 31 μg, 32 μg, 33 μg, 34 μg, 35 μg, 36 μg, 37 μg, 38 μg, 39 μg, 40 μg, 41 μg, 42 μg 43 μg, 44 μg, 45 μg, 46 μg, 47 μg, 48 μg, 49 μg, 50 μg, 51 μg, 52 μg 53 μg, 54 μg, 55 μg, 56 μg, 57 μg, 58 μg, 59 μg, 60 μg, 61 μg, 62 μg, 63 μg, 64 μg, 65 μg, 66 μg, 67 μg, 68 μg, 69 μg, 70 μg, 71 μg, 72 μg 73 μg, 74 μg, 75 μg, 76 μg, 77 μg, 78 μg, 79 μg, 80 μg, 81 μg, 82 μg 83 μg, 84 μg, 85 μg, 86 μg, 87 μg 88 μg, 89 μg, 90 μg, 91 μg, 92 μg, 93 μg, 94 μg, 95 μg, 96 μg, 97 μg, 98 μg, 99 μg, 100 μg, 101 μg, 102 μg 103 μg, 104 μg, 105 μg, 106 μg, 107 μg, 108 μg, 109 μg, 110 μg, 111 μg, 112 μg, 113 μg, 114 μg, 115 μg, 116 μg, 117 μg 118 μg, 119 μg, or 120 μg; from 9 μg to: 10 μg, 11 μg, 12 μg, 13 μg, 14 μg, 15 μg, 16 μg, 17 μg, 18 μg, 19 μg, 20 μg, 21 μg, 22 μg, 23 μg, 24 μg, 25 μg, 26 μg, 27 μg 28 μg, 29 μg, 30 μg, 31 μg, 32 μg 33 μg, 34 μg, 35 μg, 36 μg, 37 μg 38 μg, 39 μg, 40 μg, 41 μg, 42 μg, 43 μg, 44 μg, 45 μg, 46 μg, 47 μg, 48 μg, 49 μg, 50 μg, 51 μg, 52 μg, 53 μg, 54 μg, 55 μg, 56 μg, 57 μg, 58 μg, 59 μg, 60 μg, 61 μg, 62 μg, 63 μg, 64 μg, 65 μg, 66 μg, 67 μg, 68 μg, 69 μg, 70 μg, 71 μg, 72 μg 73 μg, 74 μg, 75 μg, 76 μg, 77 μg 78 μg, 79 μg, 80 μg, 81 μg, 82 μg, 83 μg, 84 μg, 85 μg, 86 μg, 87 μg 88 μg, 89 μg, 90 μg, 91 μg, 92 μg, 93 μg, 94 μg, 95 μg, 96 μg, 97 μg 98 μg, 99 μg, 100 μg, 101 μg, 102 μg, 103 μg, 104 μg, 105 μg, 106 μg, 107 μg, 108 μg, 109 μg, 110 μg, 111 μg, 112 μg, 113 μg, 114 μg, 115 μg, 116 μg, 117 μg 118 μg, 119 μg, or 120 μg; from 11 μg to: 12 μg, 13 μg, 14 μg, 15 μg, 16 μg, 17 μg 18 μg, 19 μg, 20 μg, 21 μg, 22 μg, 23 μg, 24 μg, 25 μg, 26 μg, 27 μg, 28 μg, 29 μg, 30 μg, 31 μg, 32 μg, 33 μg, 34 μg, 35 μg, 36 μg, 37 μg, 38 μg, 39 μg, 40 μg, 41 μg, 42 μg, 43 μg, 44 μg, 45 μg, 46 μg, 47 μg, 48 μg, 49 μg, 50 μg, 51 μg, 52 μg, 53 μg, 54 μg, 55 μg, 56 μg, 57 μg 58 μg, 59 μg, 60 μg, 61 μg, 62 μg 63 μg, 64 μg, 65 μg, 66 μg, 67 μg, 68 μg, 69 μg, 70 μg, 71 μg, 72 μg 73 μg, 74 μg, 75 μg, 76 μg, 77 μg, 78 μg, 79 μg, 80 μg, 81 μg, 82 μg, 83 μg, 84 μg, 85 μg, 86 μg, 87 μg, 88 μg, 89 μg, 90 μg, 91 μg, 92 μg, 93 μg, 94 μg, 95 μg, 96 μg, 97 μg, 98 μg, 99 μg, 100 μg, 101 μg, 102 μg 103 μg, 104 μg, 105 μg, 106 μg, 107 μg 108 μg, 109 μg, 110 μg, 111 μg, 112 μg 113 μg, 114 μg, 115 μg, 116 μg, 117 μg 118 μg, 119 μg, or 120 μg; from 13 μg to: 14 μg, 15 μg, 16 μg, 17 μg, 18 μg, 19 μg, 20 μg, 21 μg, 22 μg 23 μg, 24 μg, 25 μg, 26 μg, 27 μg 28 μg, 29 μg, 30 μg, 31 μg, 32 μg, 33 μg, 34 μg, 35 μg, 36 μg, 37 μg, 38 μg, 39 μg, 40 μg, 41 μg, 42 μg, 43 μg, 44 μg, 45 μg, 46 μg, 47 μg, 48 μg, 49 μg, 50 μg, 51 μg, 52 μg, 53 μg, 54 μg, 55 μg, 56 μg, 57 μg, 58 μg, 59 μg, 60 μg, 61 μg, 62 μg, 63 μg, 64 μg, 65 μg, 66 μg, 67 μg 68 μg, 69 μg, 70 μg, 71 μg, 72 μg 73 μg, 74 μg, 75 μg, 76 μg, 77 μg 78 μg, 79 μg, 80 μg, 81 μg, 82 μg, 83 μg, 84 μg, 85 μg, 86 μg, 87 μg, 88 μg, 89 μg, 90 μg, 91 μg, 92 μg, 93 μg, 94 μg, 95 μg, 96 μg, 97 μg, 98 μg, 99 μg, 100 μg, 101 μg, 102 μg 103 μg, 104 μg, 105 μg, 106 μg, 107 μg, 108 μg, 109 μg, 110 μg, 111 μg, 112 μg, 113 μg, 114 μg, 115 μg, 116 μg, 117 μg, 118 μg, 119 μg, or 120 μg; from 15 μg to: 16 μg, 17 μg 18 μg, 19 μg, 20 μg, 21 μg, 22 μg, 23 μg, 24 μg, 25 μg, 26 μg, 27 μg, 28 μg, 29 μg, 30 μg, 31 μg, 32 μg, 33 μg, 34 μg, 35 μg, 36 μg, 37 μg, 38 μg, 39 μg, 40 μg, 41 μg, 42 μg, 43 μg, 44 μg, 45 μg, 46 μg, 47 μg, 48 μg, 49 μg, 50 μg, 51 μg, 52 μg, 53 μg, 54 μg, 55 μg, 56 μg, 57 μg 58 μg, 59 μg, 60 μg, 61 μg, 62 μg 63 μg, 64 μg, 65 μg, 66 μg, 67 μg, 68 μg, 69 μg, 70 μg, 71 μg, 72 μg 73 μg, 74 μg, 75 μg, 76 μg, 77 μg, 78 μg, 79 μg, 80 μg, 81 μg, 82 μg 83 μg, 84 μg, 85 μg, 86 μg, 87 μg, 88 μg, 89 μg, 90 μg, 91 μg, 92 μg, 93 μg, 94 μg, 95 μg, 96 μg, 97 μg 98 μg, 99 μg, 100 μg, 101 μg, 102 μg, 103 μg, 104 μg, 105 μg, 106 μg, 107 μg, 108 μg, 109 μg, 110 μg, 111 μg, 112 μg 113 μg, 114 μg, 115 μg, 116 μg, 117 μg 118 μg, 119 μg, or 120 μg; from 17 μg to: 18 μg, 19 μg, 20 μg, 21 μg, 22 μg, 23 μg, 24 μg, 25 μg, 26 μg, 27 μg, 28 μg, 29 μg, 30 μg, 31 μg, 32 μg 33 μg, 34 μg, 35 μg, 36 μg, 37 μg 38 μg, 39 μg, 40 μg, 41 μg, 42 μg 43 μg, 44 μg, 45 μg, 46 μg, 47 μg 48 μg, 49 μg, 50 μg, 51 μg, 52 μg, 53 μg, 54 μg, 55 μg, 56 μg, 57 μg 58 μg, 59 μg, 60 μg, 61 μg, 62 μg 63 μg, 64 μg, 65 μg, 66 μg, 67 μg 68 μg, 69 μg, 70 μg, 71 μg, 72 μg 73 μg, 74 μg, 75 μg, 76 μg, 77 μg 78 μg, 79 μg, 80 μg, 81 μg, 82 μg, 83 μg, 84 μg, 85 μg, 86 μg, 87 μg, 88 μg, 89 μg, 90 μg, 91 μg, 92 μg, 93 μg, 94 μg, 95 μg, 96 μg, 97 μg, 98 μg, 99 μg, 100 μg, 101 μg, 102 μg 103 μg, 104 μg, 105 μg, 106 μg, 107 μg, 108 μg, 109 μg, 110 μg, 111 μg, 112 μg 113 μg, 114 μg, 115 μg, 116 μg, 117 μg, 118 μg, 119 μg, or 120 μg; from 19 μg to: 20 μg, 21 μg, 22 μg, 23 μg, 24 μg, 25 μg, 26 μg, 27 μg, 28 μg, 29 μg, 30 μg, 31 μg, 32 μg 33 μg, 34 μg, 35 μg, 36 μg, 37 μg, 38 μg, 39 μg, 40 μg, 41 μg, 42 μg 43 μg, 44 μg, 45 μg, 46 μg, 47 μg 48 μg, 49 μg, 50 μg, 51 μg, 52 μg 53 μg, 54 μg, 55 μg, 56 μg, 57 μg 58 μg, 59 μg, 60 μg, 61 μg, 62 μg, 63 μg, 64 μg, 65 μg, 66 μg, 67 μg 68 μg, 69 μg, 70 μg, 71 μg, 72 μg 73 μg, 74 μg, 75 μg, 76 μg, 77 μg 78 μg, 79 μg, 80 μg, 81 μg, 82 μg 83 μg, 84 μg, 85 μg, 86 μg, 87 μg 88 μg, 89 μg, 90 μg, 91 μg, 92 μg, 93 μg, 94 μg, 95 μg, 96 μg, 97 μg 98 μg, 99 μg, 100 μg, 101 μg, 102 μg 103 μg, 104 μg, 105 μg, 106 μg, 107 μg, 108 μg, 109 μg, 110 μg, 111 μg, 112 μg, 113 μg, 114 μg, 115 μg, 116 μg, 117 μg 118 μg, 119 μg, or 120 μg; from 21 μg to: 22 μg 23 μg, 24 μg, 25 μg, 26 μg, 27 μg 28 μg, 29 μg, 30 μg, 31 μg, 32 μg, 33 μg, 34 μg, 35 μg, 36 μg, 37 μg 38 μg, 39 μg, 40 μg, 41 μg, 42 μg, 43 μg, 44 μg, 45 μg, 46 μg, 47 μg, 48 μg, 49 μg, 50 μg, 51 μg, 52 μg 53 μg, 54 μg, 55 μg, 56 μg, 57 μg 58 μg, 59 μg, 60 μg, 61 μg, 62 μg, 63 μg, 64 μg, 65 μg, 66 μg, 67 μg 68 μg, 69 μg, 70 μg, 71 μg, 72 μg, 73 μg, 74 μg, 75 μg, 76 μg, 77 μg, 78 μg, 79 μg, 80 μg, 81 μg, 82 μg 83 μg, 84 μg, 85 μg, 86 μg, 87 μg, 88 μg, 89 μg, 90 μg, 91 μg, 92 μg, 93 μg, 94 μg, 95 μg, 96 μg, 97 μg, 98 μg, 99 μg, 100 μg, 101 μg, 102 μg 103 μg, 104 μg, 105 μg, 106 μg, 107 μg, 108 μg, 109 μg, 110 μg, 111 μg, 112 μg, 113 μg, 114 μg, 115 μg, 116 μg, 117 μg, 118 μg, 119 μg, or 120 μg; from 23 μg to: 24 μg, 25 μg, 26 μg, 27 μg 28 μg, 29 μg, 30 μg, 31 μg, 32 μg 33 μg, 34 μg, 35 μg, 36 μg, 37 μg 38 μg, 39 μg, 40 μg, 41 μg, 42 μg, 43 μg, 44 μg, 45 μg, 46 μg, 47 μg, 48 μg, 49 μg, 50 μg, 51 μg, 52 μg, 53 μg, 54 μg, 55 μg, 56 μg, 57 μg, 58 μg, 59 μg, 60 μg, 61 μg, 62 μg 63 μg, 64 μg, 65 μg, 66 μg, 67 μg, 68 μg, 69 μg, 70 μg, 71 μg, 72 μg, 73 μg, 74 μg, 75 μg, 76 μg, 77 μg 78 μg, 79 μg, 80 μg, 81 μg, 82 μg, 83 μg, 84 μg, 85 μg, 86 μg, 87 μg, 88 μg, 89 μg, 90 μg, 91 μg, 92 μg, 93 μg, 94 μg, 95 μg, 96 μg, 97 μg, 98 μg, 99 μg, 100 μg, 101 μg, 102 μg, 103 μg, 104 μg, 105 μg, 106 μg, 107 μg, 108 μg, 109 μg, 110 μg, 111 μg, 112 μg 113 μg, 114 μg, 115 μg, 116 μg, 117 μg 118 μg, 119 μg, or 120 μg; from 25 μg to: 26 μg, 27 μg, 28 μg, 29 μg, 30 μg, 31 μg, 32 μg 33 μg, 34 μg, 35 μg, 36 μg, 37 μg, 38 μg, 39 μg, 40 μg, 41 μg, 42 μg, 43 μg, 44 μg, 45 μg, 46 μg, 47 μg, 48 μg, 49 μg, 50 μg, 51 μg, 52 μg, 53 μg, 54 μg, 55 μg, 56 μg, 57 μg, 58 μg, 59 μg, 60 μg, 61 μg, 62 μg 63 μg, 64 μg, 65 μg, 66 μg, 67 μg, 68 μg, 69 μg, 70 μg, 71 μg, 72 μg 73 μg, 74 μg, 75 μg, 76 μg, 77 μg 78 μg, 79 μg, 80 μg, 81 μg, 82 μg, 83 μg, 84 μg, 85 μg, 86 μg, 87 μg, 88 μg, 89 μg, 90 μg, 91 μg, 92 μg, 93 μg, 94 μg, 95 μg, 96 μg, 97 μg, 98 μg, 99 μg, 100 μg, 101 μg, 102 μg, 103 μg, 104 μg, 105 μg, 106 μg, 107 μg, 108 μg, 109 μg, 110 μg, 111 μg, 112 μg 113 μg, 114 μg, 115 μg, 116 μg, 117 μg 118 μg, 119 μg, or 120 μg; from 27 μg to: 28 μg, 29 μg, 30 μg, 31 μg, 32 μg, 33 μg, 34 μg, 35 μg, 36 μg, 37 μg, 38 μg, 39 μg, 40 μg, 41 μg, 42 μg, 43 μg, 44 μg, 45 μg, 46 μg, 47 μg 48 μg, 49 μg, 50 μg, 51 μg, 52 μg, 53 μg, 54 μg, 55 μg, 56 μg, 57 μg 58 μg, 59 μg, 60 μg, 61 μg, 62 μg 63 μg, 64 μg, 65 μg, 66 μg, 67 μg, 68 μg, 69 μg, 70 μg, 71 μg, 72 μg, 73 μg, 74 μg, 75 μg, 76 μg, 77 μg 78 μg, 79 μg, 80 μg, 81 μg, 82 μg, 83 μg, 84 μg, 85 μg, 86 μg, 87 μg 88 μg, 89 μg, 90 μg, 91 μg, 92 μg, 93 μg, 94 μg, 95 μg, 96 μg, 97 μg, 98 μg, 99 μg, 100 μg, 101 μg, 102 μg, 103 μg, 104 μg, 105 μg, 106 μg, 107 μg, 108 μg, 109 μg, 110 μg, 111 μg, 112 μg, 113 μg, 114 μg, 115 μg, 116 μg, 117 μg, 118 μg, 119 μg, or 120 μg; from 29 μg to: 30 μg, 31 μg, 32 μg, 33 μg, 34 μg, 35 μg, 36 μg, 37 μg 38 μg, 39 μg, 40 μg, 41 μg, 42 μg, 43 μg, 44 μg, 45 μg, 46 μg, 47 μg 48 μg, 49 μg, 50 μg, 51 μg, 52 μg, 53 μg, 54 μg, 55 μg, 56 μg, 57 μg, 58 μg, 59 μg, 60 μg, 61 μg, 62 μg 63 μg, 64 μg, 65 μg, 66 μg, 67 μg 68 μg, 69 μg, 70 μg, 71 μg, 72 μg, 73 μg, 74 μg, 75 μg, 76 μg, 77 μg 78 μg, 79 μg, 80 μg, 81 μg, 82 μg 83 μg, 84 μg, 85 μg, 86 μg, 87 μg, 88 μg, 89 μg, 90 μg, 91 μg, 92 μg, 93 μg, 94 μg, 95 μg, 96 μg, 97 μg, 98 μg, 99 μg, 100 μg, 101 μg, 102 μg 103 μg, 104 μg, 105 μg, 106 μg, 107 μg, 108 μg, 109 μg, 110 μg, 111 μg, 112 μg, 113 μg, 114 μg, 115 μg, 116 μg, 117 μg, 118 μg, 119 μg, or 120 μg; from 31 μg to: 32 μg, 33 μg, 34 μg, 35 μg, 36 μg, 37 μg, 38 μg, 39 μg, 40 μg, 41 μg, 42 μg, 43 μg, 44 μg, 45 μg, 46 μg, 47 μg, 48 μg, 49 μg, 50 μg, 51 μg, 52 μg, 53 μg, 54 μg, 55 μg, 56 μg, 57 μg, 58 μg, 59 μg, 60 μg, 61 μg, 62 μg 63 μg, 64 μg, 65 μg, 66 μg, 67 μg, 68 μg, 69 μg, 70 μg, 71 μg, 72 μg 73 μg, 74 μg, 75 μg, 76 μg, 77 μg, 78 μg, 79 μg, 80 μg, 81 μg, 82 μg, 83 μg, 84 μg, 85 μg, 86 μg, 87 μg, 88 μg, 89 μg, 90 μg, 91 μg, 92 μg, 93 μg, 94 μg, 95 μg, 96 μg, 97 μg, 98 μg, 99 μg, 100 μg, 101 μg, 102 μg 103 μg, 104 μg, 105 μg, 106 μg, 107 μg, 108 μg, 109 μg, 110 μg, 111 μg, 112 μg 113 μg, 114 μg, 115 μg, 116 μg, 117 μg, 118 μg, 119 μg, or 120 μg; from 33 μg to: 34 μg, 35 μg, 36 μg, 37 μg, 38 μg, 39 μg, 40 μg, 41 μg, 42 μg 43 μg, 44 μg, 45 μg, 46 μg, 47 μg, 48 μg, 49 μg, 50 μg, 51 μg, 52 μg, 53 μg, 54 μg, 55 μg, 56 μg, 57 μg, 58 μg, 59 μg, 60 μg, 61 μg, 62 μg 63 μg, 64 μg, 65 μg, 66 μg, 67 μg, 68 μg, 69 μg, 70 μg, 71 μg, 72 μg, 73 μg, 74 μg, 75 μg, 76 μg, 77 μg, 78 μg, 79 μg, 80 μg, 81 μg, 82 μg, 83 μg, 84 μg, 85 μg, 86 μg, 87 μg, 88 μg, 89 μg, 90 μg, 91 μg, 92 μg, 93 μg, 94 μg, 95 μg, 96 μg, 97 μg, 98 μg, 99 μg, 100 μg, 101 μg, 102 μg, 103 μg, 104 μg, 105 μg, 106 μg, 107 μg, 108 μg, 109 μg, 110 μg, 111 μg, 112 μg, 113 μg, 114 μg, 115 μg, 116 μg, 1174 μg, 118 μg, 119 μg, or 120 μg; from 35 μg to: 36 μg, 37 μg 38 μg, 39 μg, 40 μg, 41 μg, 42 μg, 43 μg, 44 μg, 45 μg, 46 μg, 47 μg, 48 μg, 49 μg, 50 μg, 51 μg, 52 μg 53 μg, 54 μg, 55 μg, 56 μg, 57 μg, 58 μg, 59 μg, 60 μg, 61 μg, 62 μg 63 μg, 64 μg, 65 μg, 66 μg, 67 μg 68 μg, 69 μg, 70 μg, 71 μg, 72 μg, 73 μg, 74 μg, 75 μg, 76 μg, 77 μg 78 μg, 79 μg, 80 μg, 81 μg, 82 μg, 83 μg, 84 μg, 85 μg, 86 μg, 87 μg, 88 μg, 89 μg, 90 μg, 91 μg, 92 μg, 93 μg, 94 μg, 95 μg, 96 μg, 97 μg, 98 μg, 99 μg, 100 μg, 101 μg, 102 μg 103 μg, 104 μg, 105 μg, 106 μg, 107 μg 108 μg, 109 μg, 110 μg, 111 μg, 112 μg, 113 μg, 114 μg, 115 μg, 116 μg, 117 μg 118 μg, 119 μg, or 120 μg; from 37 μg to: 38 μg, 39 μg, 40 μg, 41 μg, 42 μg, 43 μg, 44 μg, 45 μg, 46 μg, 47 μg, 48 μg, 49 μg, 50 μg, 51 μg, 52 μg, 53 μg, 54 μg, 55 μg, 56 μg, 57 μg 58 μg, 59 μg, 60 μg, 61 μg, 62 μg, 63 μg, 64 μg, 65 μg, 66 μg, 67 μg, 68 μg, 69 μg, 70 μg, 71 μg, 72 μg 73 μg, 74 μg, 75 μg, 76 μg, 77 μg, 78 μg, 79 μg, 80 μg, 81 μg, 82 μg, 83 μg, 84 μg, 85 μg, 86 μg, 87 μg, 88 μg, 89 μg, 90 μg, 91 μg, 92 μg, 93 μg, 94 μg, 95 μg, 96 μg, 97 μg, 98 μg, 99 μg, 100 μg, 101 μg, 102 μg, 103 μg, 104 μg, 105 μg, 106 μg, 107 μg, 108 μg, 109 μg, 110 μg, 111 μg, 112 μg, 113 μg, 114 μg, 115 μg, 116 μg, 117 μg, 118 μg, 119 μg, or 120 μg; from 39 μg to: 40 μg, 41 μg, 42 μg, 43 μg, 44 μg, 45 μg, 46 μg, 47 μg, 48 μg, 49 μg, 50 μg, 51 μg, 52 μg 53 μg, 54 μg, 55 μg, 56 μg, 57 μg 58 μg, 59 μg, 60 μg, 61 μg, 62 μg, 63 μg, 64 μg, 65 μg, 66 μg, 67 μg, 68 μg, 69 μg, 70 μg, 71 μg, 72 μg, 73 μg, 74 μg, 75 μg, 76 μg, 77 μg 78 μg, 79 μg, 80 μg, 81 μg, 82 μg 83 μg, 84 μg, 85 μg, 86 μg, 87 μg, 88 μg, 89 μg, 90 μg, 91 μg, 92 μg, 93 μg, 94 μg, 95 μg, 96 μg, 97 μg, 98 μg, 99 μg, 100 μg, 101 μg, 102 μg 103 μg, 104 μg, 105 μg, 106 μg, 107 μg, 108 μg, 109 μg, 110 μg, 111 μg, 112 μg, 113 μg, 114 μg, 115 μg, 116 μg, 117 μg, 118 μg, 119 μg, or 120 μg; from 41 μg to: 41 μg, 42 μg, 43 μg, 44 μg, 45 μg, 46 μg, 47 μg, 48 μg, 49 μg, 50 μg, 51 μg, 52 μg, 53 μg, 54 μg, 55 μg, 56 μg, 57 μg, 58 μg, 59 μg, 60 μg, 61 μg, 62 μg 63 μg, 64 μg, 65 μg, 66 μg, 67 μg, 68 μg, 69 μg, 70 μg, 71 μg, 72 μg, 73 μg, 74 μg, 75 μg, 76 μg, 77 μg, 78 μg, 79 μg, 80 μg, 81 μg, 82 μg, 83 μg, 84 μg, 85 μg, 86 μg, 87 μg, 88 μg, 89 μg, 90 μg, 91 μg, 92 μg, 93 μg, 94 μg, 95 μg, 96 μg, 97 μg, 98 μg, 99 μg, 100 μg, 101 μg, 102 μg, 103 μg, 104 μg, 105 μg, 106 μg, 107 μg, 108 μg, 109 μg, 110 μg, 111 μg, 112 μg 113 μg, 114 μg, 115 μg, 116 μg, 117 μg, 118 μg, 119 μg, or 120 μg; from 43 μg to: 44 μg, 45 μg, 46 μg, 47 μg, 48 μg, 49 μg, 50 μg, 51 μg, 52 μg 53 μg, 54 μg, 55 μg, 56 μg, 57 μg, 58 μg, 59 μg, 60 μg, 61 μg, 62 μg, 63 μg, 64 μg, 65 μg, 66 μg, 67 μg, 68 μg, 69 μg, 70 μg, 71 μg, 72 μg, 73 μg, 74 μg, 75 μg, 76 μg, 77 μg, 78 μg, 79 μg, 80 μg, 81 μg, 82 μg, 83 μg, 84 μg, 85 μg, 86 μg, 87 μg, 88 μg, 89 μg, 90 μg, 91 μg, 92 μg, 93 μg, 94 μg, 95 μg, 96 μg, 97 μg, 98 μg, 99 μg, 100 μg, 101 μg, 102 μg, 103 μg, 104 μg, 105 μg, 106 μg, 107 μg, 108 μg, 109 μg, 110 μg, 111 μg, 112 μg 113 μg, 114 μg, 115 μg, 116 μg, 117 μg, 118 μg, 119 μg, or 120 μg; from 45 μg to: 46 μg, 47 μg, 48 μg, 49 μg, 50 μg, 51 μg, 52 μg 53 μg, 54 μg, 55 μg, 56 μg, 57 μg, 58 μg, 59 μg, 60 μg, 61 μg, 62 μg, 63 μg, 64 μg, 65 μg, 66 μg, 67 μg, 68 μg, 69 μg, 70 μg, 71 μg, 72 μg, 73 μg, 74 μg, 75 μg, 76 μg, 77 μg, 78 μg, 79 μg, 80 μg, 81 μg, 82 μg 83 μg, 84 μg, 85 μg, 86 μg, 87 μg, 88 μg, 89 μg, 90 μg, 91 μg, 92 μg, 93 μg, 94 μg, 95 μg, 96 μg, 97 μg, 98 μg, 99 μg, 100 μg, 101 μg, 102 μg 103 μg, 104 μg, 105 μg, 106 μg, 107 μg, 108 μg, 109 μg, 110 μg, 111 μg, 112 μg, 113 μg, 114 μg, 115 μg, 116 μg, 117 μg, 118 μg, 119 μg, or 120 μg; from 47 μg to: 48 μg, 49 μg, 50 μg, 51 μg, 52 μg 53 μg, 54 μg, 55 μg, 56 μg, 57 μg, 58 μg, 59 μg, 60 μg, 61 μg, 62 μg, 63 μg, 64 μg, 65 μg, 66 μg, 67 μg, 68 μg, 69 μg, 70 μg, 71 μg, 72 μg, 73 μg, 74 μg, 75 μg, 76 μg, 77 μg, 78 μg, 79 μg, 80 μg, 81 μg, 82 μg 83 μg, 84 μg, 85 μg, 86 μg, 87 μg, 88 μg, 89 μg, 90 μg, 91 μg, 92 μg 93 μg, 94 μg, 95 μg, 96 μg, 97 μg, 98 μg, 99 μg, 100 μg, 101 μg, 102 μg, 103 μg, 104 μg, 105 μg, 106 μg, 107 μg, 108 μg, 109 μg, 110 μg, 111 μg, 112 μg, 113 μg, 114 μg, 115 μg, 116 μg, 117 μg 118 μg, 119 μg, or 120 μg; from 49 μg to: 50 μg, 51 μg, 52 μg 53 μg, 54 μg, 55 μg, 56 μg, 57 μg, 58 μg, 59 μg, 60 μg, 61 μg, 62 μg 63 μg, 64 μg, 65 μg, 66 μg, 67 μg 68 μg, 69 μg, 70 μg, 71 μg, 72 μg, 73 μg, 74 μg, 75 μg, 76 μg, 77 μg, 78 μg, 79 μg, 80 μg, 81 μg, 82 μg, 83 μg, 84 μg, 85 μg, 86 μg, 87 μg 88 μg, 89 μg, 90 μg, 91 μg, 92 μg 93 μg, 94 μg, 95 μg, 96 μg, 97 μg, 98 μg, 99 μg, 100 μg, 101 μg, 102 μg, 103 μg, 104 μg, 105 μg, 106 μg, 107 μg, 108 μg, 109 μg, 110 μg, 111 μg, 112 μg, 113 μg, 114 μg, 115 μg, 116 μg, 117 μg, 118 μg, 119 μg, or 120 μg; from 51 μg to: 52 μg, 53 μg, 54 μg, 55 μg, 56 μg, 57 μg 58 μg, 59 μg, 60 μg, 61 μg, 62 μg 63 μg, 64 μg, 65 μg, 66 μg, 67 μg 68 μg, 69 μg, 70 μg, 71 μg, 72 μg 73 μg, 74 μg, 75 μg, 76 μg, 77 μg, 78 μg, 79 μg, 80 μg, 81 μg, 82 μg, 83 μg, 84 μg, 85 μg, 86 μg, 87 μg, 88 μg, 89 μg, 90 μg, 91 μg, 92 μg, 93 μg, 94 μg, 95 μg, 96 μg, 97 μg, 98 μg, 99 μg, 100 μg, 101 μg, 102 μg, 103 μg, 104 μg, 105 μg, 106 μg, 107 μg, 108 μg, 109 μg, 110 μg, 111 μg, 112 μg 113 μg, 114 μg, 115 μg, 116 μg, 117 μg, 118 μg, 119 μg, or 120 μg; from 53 μg to: 54 μg, 55 μg, 56 μg, 57 μg 58 μg, 59 μg, 60 μg, 61 μg, 62 μg, 63 μg, 64 μg, 65 μg, 66 μg, 67 μg, 68 μg, 69 μg, 70 μg, 71 μg, 72 μg, 73 μg, 74 μg, 75 μg, 76 μg, 77 μg, 78 μg, 79 μg, 80 μg, 81 μg, 82 μg 83 μg, 84 μg, 85 μg, 86 μg, 87 μg, 88 μg, 89 μg, 90 μg, 91 μg, 92 μg, 93 μg, 94 μg, 95 μg, 96 μg, 97 μg, 98 μg, 99 μg, 100 μg, 101 μg, 102 μg, 103 μg, 104 μg, 105 μg, 106 μg, 107 μg, 108 μg, 109 μg, 110 μg, 111 μg, 112 μg, 113 μg, 114 μg, 115 μg, 116 μg, 117 μg, 118 μg, 119 μg, or 120 μg; from 55 μg to: 56 μg, 57 μg, 58 μg, 59 μg, 60 μg, 61 μg, 62 μg, 63 μg, 64 μg, 65 μg, 66 μg, 67 μg, 68 μg, 69 μg, 70 μg, 71 μg, 72 μg, 73 μg, 74 μg, 75 μg, 76 μg, 77 μg 78 μg, 79 μg, 80 μg, 81 μg, 82 μg 83 μg, 84 μg, 85 μg, 86 μg, 87 μg, 88 μg, 89 μg, 90 μg, 91 μg, 92 μg, 93 μg, 94 μg, 95 μg, 96 μg, 97 μg, 98 μg, 99 μg, 100 μg, 101 μg, 102 μg, 103 μg, 104 μg, 105 μg, 106 μg, 107 μg, 108 μg, 109 μg, 110 μg, 111 μg, 112 μg, 113 μg, 114 μg, 115 μg, 116 μg, 117 μg, 118 μg, 119 μg, or 120 μg; from 57 μg to: 58 μg, 59 μg, 60 μg, 61 μg, 62 μg 63 μg, 64 μg, 65 μg, 66 μg, 67 μg, 68 μg, 69 μg, 70 μg, 71 μg, 72 μg, 73 μg, 74 μg, 75 μg, 76 μg, 77 μg, 78 μg, 79 μg, 80 μg, 81 μg, 82 μg, 83 μg, 84 μg, 85 μg, 86 μg, 87 μg, 88 μg, 89 μg, 90 μg, 91 μg, 92 μg, 93 μg, 94 μg, 95 μg, 96 μg, 97 μg, 98 μg, 99 μg, 100 μg, 101 μg, 102 μg 103 μg, 104 μg, 105 μg, 106 μg, 107 μg, 108 μg, 109 μg, 110 μg, 111 μg, 112 μg, 113 μg, 114 μg, 115 μg, 116 μg, 117 μg, 118 μg, 119 μg, or 120 μg; from 59 μg to: 60 μg, 61 μg, 62 μg, 63 μg, 64 μg, 65 μg, 66 μg, 67 μg, 68 μg, 69 μg, 70 μg, 71 μg, 72 μg, 73 μg, 74 μg, 75 μg, 76 μg, 77 μg, 78 μg, 79 μg, 80 μg, 81 μg, 82 μg, 83 μg, 84 μg, 85 μg, 86 μg, 87 μg, 88 μg, 89 μg, 90 μg, 91 μg, 92 μg, 93 μg, 94 μg, 95 μg, 96 μg, 97 μg, 98 μg, 99 μg, 100 μg, 101 μg, 102 μg, 103 μg, 104 μg, 105 μg, 106 μg, 107 μg, 108 μg, 109 μg, 110 μg, 111 μg, 112 μg, 113 μg, 114 μg, 115 μg, 116 μg, 117 μg, 118 μg, 119 μg, or 120 μg; from 61 μg to: 62 μg, 63 μg, 64 μg, 65 μg, 66 μg, 67 μg, 68 μg, 69 μg, 70 μg, 71 μg, 72 μg, 73 μg, 74 μg, 75 μg, 76 μg, 77 μg, 78 μg, 79 μg, 80 μg, 81 μg, 82 μg 83 μg, 84 μg, 85 μg, 86 μg, 87 μg, 88 μg, 89 μg, 90 μg, 91 μg, 92 μg, 93 μg, 94 μg, 95 μg, 96 μg, 97 μg, 98 μg, 99 μg, 100 μg, 101 μg, 102 μg, 103 μg, 104 μg, 105 μg, 106 μg, 107 μg, 108 μg, 109 μg, 110 μg, 111 μg, 112 μg, 113 μg, 114 μg, 115 μg, 116 μg, 117 μg, 118 μg, 119 μg, or 120 μg; from 63 μg to: 64 μg, 65 μg, 66 μg, 67 μg, 68 μg, 69 μg, 70 μg, 71 μg, 72 μg 73 μg, 74 μg, 75 μg, 76 μg, 77 μg, 78 μg, 79 μg, 80 μg, 81 μg, 82 μg, 83 μg, 84 μg, 85 μg, 86 μg, 87 μg, 88 μg, 89 μg, 90 μg, 91 μg, 92 μg 93 μg, 94 μg, 95 μg, 96 μg, 97 μg, 98 μg, 99 μg, 100 μg, 101 μg, 102 μg 103 μg, 104 μg, 105 μg, 106 μg, 107 μg 108 μg, 109 μg, 110 μg, 111 μg, 112 μg 113 μg, 114 μg, 115 μg, 116 μg, 1174 μg 118 μg, 119 μg, or 120 μg; from 65 μg to: 66 μg, 67 μg, 68 μg, 69 μg, 70 μg, 71 μg, 72 μg, 73 μg, 74 μg, 75 μg, 76 μg, 77 μg, 78 μg, 79 μg, 80 μg, 81 μg, 82 μg, 83 μg, 84 μg, 85 μg, 86 μg, 87 μg, 88 μg, 89 μg, 90 μg, 91 μg, 92 μg, 93 μg, 94 μg, 95 μg, 96 μg, 97 μg, 98 μg, 99 μg, 100 μg, 101 μg, 102 μg, 103 μg, 104 μg, 105 μg, 106 μg, 107 μg, 108 μg, 109 μg, 110 μg, 111 μg, 112 μg 113 μg, 114 μg, 115 μg, 116 μg, 117 μg, 118 μg, 119 μg, or 120 μg; from 67 μg to: 68 μg, 69 μg, 70 μg, 71 μg, 72 μg, 73 μg, 74 μg, 75 μg, 76 μg, 77 μg, 78 μg, 79 μg, 80 μg, 81 μg, 82 μg 83 μg, 84 μg, 85 μg, 86 μg, 87 μg, 88 μg, 89 μg, 90 μg, 91 μg, 92 μg, 93 μg, 94 μg, 95 μg, 96 μg, 97 μg, 98 μg, 99 μg, 100 μg, 101 μg, 102 μg, 103 μg, 104 μg, 105 μg, 106 μg, 107 μg, 108 μg, 109 μg, 110 μg, l 111 μg, 112 μg, 113 μg, 114 μg, 115 μg, 116 μg, 117 μg, 118 μg, 119 μg, or 120 μg; from 69 μg to: 70 μg, 71 μg, 72 μg 73 μg, 74 μg, 75 μg, 76 μg, 77 μg, 78 μg, 79 μg, 80 μg, 81 μg, 82 μg, 83 μg, 84 μg, 85 μg, 86 μg, 87 μg, 88 μg, 89 μg, 90 μg, 91 μg, 92 μg, 93 μg, 94 μg, 95 μg, 96 μg, 97 μg, 98 μg, 99 μg, 100 μg, 101 μg, 102 μg, 103 μg, 104 μg, 105 μg, 106 μg, 107 μg, 108 μg, 109 μg, 110 μg, 111 μg, 112 μg 113 μg, 114 μg, 115 μg, 116 μg, 117 μg, 118 μg, 119 μg, or 120 μg; from 71 μg to: 72 μg, 73 μg, 74 μg, 75 μg, 76 μg, 77 μg, 78 μg, 79 μg, 80 μg, 81 μg, 82 μg, 83 μg, 84 μg, 85 μg, 86 μg, 87 μg, 88 μg, 89 μg, 90 μg, 91 μg, 92 μg 93 μg, 94 μg, 95 μg, 96 μg, 97 μg, 98 μg, 99 μg, 100 μg, 101 μg, 102 μg 103 μg, 104 μg, 105 μg, 106 μg, 107 μg, 108 μg, 109 μg, 110 μg, 111 μg, 112 μg, 113 μg, 114 μg, 115 μg, 116 μg, 117 μg, 118 μg, 119 μg, or 120 μg; from 73 μg to: 74 μg, 75 μg, 76 μg, 77 μg, 78 μg, 79 μg, 80 μg, 81 μg, 82 μg, 83 μg, 84 μg, 85 μg, 86 μg, 87 μg, 88 μg, 89 μg, 90 μg, 91 μg, 92 μg, 93 μg, 94 μg, 95 μg, 96 μg, 97 μg, 98 μg, 99 μg, 100 μg, 101 μg, 102 μg, 103 μg, 104 μg, 105 μg, 106 μg, 107 μg, 108 μg, 109 μg, 110 μg, 111 μg, 112 μg 113 μg, 114 μg, 115 μg, 116 μg, 117 μg, 118 μg, 119 μg, or 120 μg; from 75 μg to: 76 μg, 77 μg, 78 μg, 79 μg, 80 μg, 81 μg, 82 μg, 83 μg, 84 μg, 85 μg, 86 μg, 87 μg, 88 μg, 89 μg, 90 μg, 91 μg, 92 μg 93 μg, 94 μg, 95 μg, 96 μg, 97 μg, 98 μg, 99 μg, 100 μg, 101 μg, 102 μg, 103 μg, 104 μg, 105 μg, 106 μg, 107 μg, 108 μg, 109 μg, 110 μg, 111 μg, 112 μg 113 μg, 114 μg, 115 μg, 116 μg, 117 μg, 118 μg, 119 μg, or 120 μg; from 77 μg to: 78 μg, 79 μg, 80 μg, 81 μg, 82 μg 83 μg, 84 μg, 85 μg, 86 μg, 87 μg, 88 μg, 89 μg, 90 μg, 91 μg, 92 μg 93 μg, 94 μg, 95 μg, 96 μg, 97 μg, 98 μg, 99 μg, 100 μg, 101 μg, 102 μg, 103 μg, 104 μg, 105 μg, 106 μg, 107 μg, 108 μg, 109 μg, 110 μg, 111 μg, 112 μg 113 μg, 114 μg, 115 μg, 116 μg, 117 μg, 118 μg, 119 μg, or 120 μg; or from 79 μg to: 80 μg, 81 μg, 82 μg, 83 μg, 84 μg, 85 μg, 86 μg, 87 μg, 88 μg, 89 μg, 90 μg, 91 μg, 92 μg, 93 μg, 94 μg, 95 μg, 96 μg, 97 μg, 98 μg, 99 μg, 100 μg, 101 μg, 102 μg, 103 μg, 104 μg, 105 μg, 106 μg, 107 μg, 108 μg, 109 μg, 110 μg, 111 μg, 112 μg, 113 μg, 114 μg, 115 μg, 116 μg, 117 μg, 118 μg, 119 μg, or 120 μg of recombinant RNA molecules.

Also contemplated and supported are combinations of unit doses of “at least” and any of the above-noted unit doses of “no more than” with the unit doses provided in an Example of this document. Also contemplated and supported are combinations of the unit doses of the Example section of this document to provide a range (i.e. the unit dose from [the unit dose from example X₁] to [the unit dose from example X₂] wherein X₁ and X₂ represent any two exemplary treatments of the Examples.

Embodiments are further described in the following numbered paragraphs:
1. A pharmaceutical composition
said pharmaceutical composition comprising a nucleic acid and lipid carrier particles
wherein the pharmaceutical composition is lyophilised and wherein either
(a) the percentage of nucleic acid that is encapsulated within the lipid carrier particles is greater than 75% of the total nucleic acid in the pharmaceutical composition; and/or
(b) the percentage of nucleic acid that remains encapsulated within the lipid carrier particles is reduced by less than 15% compared to the percentage of nucleic acid that is encapsulated within the lipid carrier particles in the pharmaceutical composition prior to lyophilisation.
2. The pharmaceutical composition of paragraph 1 wherein (a) the percentage of nucleic acid that is encapsulated within the lipid carrier particles is greater than 77.5%, greater than 80%, greater than 82.5%, greater than 85%, greater than 87.5%, greater than 90%, greater than 92.5%, greater than 95% or greater than 97.5% compared to total nucleic acid in the pharmaceutical composition.
3. The pharmaceutical composition of paragraph 1 wherein (b) the percentage of nucleic acid that remains encapsulated within the lipid carrier particles is reduced by less than 12.5%, less than 10%, less than 7.5%, less than 5% or less than 2.5% compared to the percentage of nucleic acid that is encapsulated within the lipid carrier particles in the pharmaceutical composition prior to lyophilisation.
4. The pharmaceutical composition of paragraphs 1-3 wherein the lyophilised pharmaceutical composition comprises a polydispersity index (PDI) of less than 0.8, less than 0.5 or less than 0.2 as assessed by dynamic light scattering (DLS) analysis.
5. The pharmaceutical composition of paragraph 4 wherein the lyophilised pharmaceutical composition comprises a PDI of between 0.1 and 0.25 as assessed by DLS analysis.
6. The pharmaceutical composition of any preceding paragraph wherein the percentage of nucleic acid that is encapsulated within the lipid carrier particles and/or the PDI is assessed following reconstitution of the lyophilised pharmaceutical composition.
7. The pharmaceutical composition of paragraph 6 wherein the lyophilised pharmaceutical composition is reconstituted with sterile water, saline, or liquid adjuvant.
8. The pharmaceutical composition of any preceding paragraph wherein the percentage of nucleic acid that is encapsulated within the lipid carrier particles is determined by comparing the signal of a nucleic acid-binding fluorescent dye in the absence and presence of a detergent, wherein in the absence of a detergent, the signal comes only from unencapsulated nucleic acid and wherein in the presence of a detergent, the lipid carrier particle is disrupted so that the signal comes from the total nucleic acid (both encapsulated and non-encapsulated).
9. The pharmaceutical composition of paragraph 8 wherein the nucleic acid-binding dye is Ribogreen.
10. The pharmaceutical composition of paragraph 8 wherein the detergent is Triton X-100, optionally at a concentration of between 0.01% and 1% (w/v), optionally at a concentration of around 0.1% (w/v).
11. The pharmaceutical composition of any preceding claim wherein said pharmaceutical composition is contained within a vessel.
12. The pharmaceutical composition of paragraph 11 wherein the lyophilised pharmaceutical composition is coated in a layer of substantially uniform thickness against an inner surface of a circumferential wall of the vessel.
13. The pharmaceutical composition of paragraph 12 wherein greater than 80%, greater than 82.5%, greater than 85%, greater than 87.5%, greater than 90%, greater than 92.5%, greater than 95% or greater than 97.5% of the lyophilised pharmaceutical composition is coated in a layer of substantially uniform thickness against the inner surface of the circumferential wall of the vessel.
14. The pharmaceutical composition of paragraph 12 or paragraph 13 wherein the pharmaceutical composition is not substantially coated on a bottom part of the vessel.
15. The pharmaceutical composition of any preceding paragraph wherein the nucleic acid is RNA.
16. The pharmaceutical composition of paragraph 15 wherein the RNA is present at a concentration of between 5-250 μg/ml, 10-100 μg/ml, 25-100 μg/ml, 25-75 μg/ml, or 50-75 μg/ml.
17. The pharmaceutical composition of paragraph 23 wherein the RNA is present at a concentration of 60 μg/ml.
18. The pharmaceutical composition of paragraphs 15-17 wherein the RNA comprises one or more modified ribonucleotides, optionally wherein said modified ribonucleotides comprise N1-methylpseudouridine (1mΨ).
19. The pharmaceutical composition of paragraph 15-18 wherein the RNA is mRNA.
20. The pharmaceutical composition of paragraph 19 wherein the mRNA encodes for at least one protein, optionally a protein immunogen.
21. The pharmaceutical composition of paragraph 19 or paragraph 20 wherein the mRNA is greater than 2500, greater than 3000, greater than 3500, greater than 4000, greater than 4500 or greater than 5000 ribonucleotides in length.
22. The pharmaceutical composition of paragraphs 19-21 wherein the mRNA is self-amplifying mRNA (SAM).
23. The pharmaceutical composition of any preceding paragraph wherein the lipid carrier particles are lipid nanoparticles (LNPs).
24. The pharmaceutical composition of any preceding paragraph wherein the nucleic acid and lipid carrier particles are RNA-LNPs, optionally mRNA-LNPs.
25. The pharmaceutical composition of paragraph 23 or paragraph 24 wherein the LNPs comprise a cationic lipid, a neutral lipid, and a PEGylated lipid.
26. The pharmaceutical composition of paragraph 25 wherein the cationic lipid is RV39.
27. The pharmaceutical composition of paragraph 25 wherein the neutral lipid is DSPC.
28. The pharmaceutical composition of paragraph 25 wherein the PEGylated lipid is present from 0.5 to 5% (w/v).
29. The pharmaceutical composition of paragraph 28 wherein the PEGylated lipid is present from 1 to 5% (w/v), optionally 2% (w/v)
30. The pharmaceutical composition of paragraphs 25-29 wherein the LNPs further comprise cholesterol.
31. The pharmaceutical composition of paragraph 30 wherein the cholesterol is present from 40% to 60% (w/v), optionally 45% to 55%, optionally 45% to 50% (w/v).
32. The pharmaceutical composition of any preceding paragraph wherein the pharmaceutical composition is lyophilised from an aqueous composition comprising said nucleic acid and lipid carrier particles.
33. The pharmaceutical composition of paragraph 32 wherein the aqueous composition further comprises sucrose in a concentration of greater than or equal to 5% (w/v).
34. The pharmaceutical composition of paragraph 33, wherein the aqueous composition comprises sucrose in a concentration of between 5% and 30% (w/v), 5% and 20% (w/v) or 5% and 10% (w/v).
35. The pharmaceutical composition of paragraph 33 or paragraph 34, wherein the aqueous composition comprises sucrose in a concentration of 7.5% (w/v).
36. The pharmaceutical composition of paragraphs 32-35 wherein the aqueous composition further comprises a salt, optionally sodium chloride.
37. The pharmaceutical composition of paragraph 36 wherein the aqueous composition comprises sodium chloride in a concentration of between 0.1 and 50 mM, 0.5 and 40 mM, 1 and 30 mM, 1 and 25 mM, 1 and 10 mM, 1 and 7.5 mM or 2.5 and 7.5 mM.
38. The pharmaceutical composition of paragraph 36 or paragraph 37 wherein the aqueous composition comprises sodium chloride in a concentration of between 1 and 20 mM, 1 and 10 mM, 2 and 8 mM or 4 and 6 mM.
39. The pharmaceutical composition of paragraphs 36-38 wherein the aqueous composition comprises sodium chloride in a concentration of 5 mM.
40. The pharmaceutical composition of any of paragraphs 32-39, wherein the aqueous composition further comprises a buffer
41. The pharmaceutical composition of paragraph 40 wherein the buffer is Tris.
42. The pharmaceutical composition of paragraph 41 wherein the concentration of Tris is between 15 and 25 mM, optionally 20 mM.
43. The pharmaceutical composition of paragraph 32-42 wherein the pH of the aqueous composition ranges from 6 to 10.
44. The pharmaceutical composition of paragraph 43 wherein the pH of the aqueous composition is 8.
45. The pharmaceutical composition of any preceding claim wherein the lyophilised pharmaceutical composition comprises a residual moisture content of between 0.25 and 2.5% (w/w), between 0.5 and 2% (w/w), between 0.5 and 1.25% (w/w), or between 0.5 and 1% (w/w).
46. A vaccine comprising the pharmaceutical composition of any of paragraph 1-45.
47. A method of reconstituting the pharmaceutical composition of any of paragraphs 1-45, comprising adding a sterile aqueous reconstitution solution to the pharmaceutical composition; and reconstituting the pharmaceutical composition.
48. A kit comprising the pharmaceutical composition of paragraphs 1-45 or the vaccine of paragraph 46, the kit comprising
a first container comprising the pharmaceutical composition of paragraphs 1-45 or the vaccine of paragraph 46 and
a second container comprising a sterile aqueous reconstitution solution.
49. The kit of paragraph 48 wherein the sterile aqueous reconstitution solution is sterile water, saline or liquid adjuvant.
50. The kit of paragraph 48 or paragraph 49 further comprise a sterile needle for injecting the pharmaceutical composition or vaccine
51. A method for producing the pharmaceutical composition of paragraphs 1-45 said method comprising
i) increasing the surface area to volume ratio of an aqueous composition comprising nucleic acid and lipid carrier particles,
ii) subjecting the aqueous composition to freezing conditions sufficient to freeze the aqueous composition, and
iii) drying the frozen composition.
52. The method of paragraph 51 wherein step ii) and step iii) occur substantially simultaneously such that increasing the surface area to volume ratio of the aqueous composition occurs whilst subjecting the aqueous composition said freezing conditions.
53. The method of paragraph 51 or paragraph 52 wherein prior to step i) the aqueous composition is formulated and transferred into a vessel(s).
54. The method of paragraph 53 wherein between 0.1 and 1.5 mL, 0.1 and 1 mL, 0.25 and 1 mL, 0.4 and 1 mL, 0.4 and 0.8 mL, 0.5 and 0.8 mL, 0.5 and 0.75 mL, 0.6 and 0.8 mL or 0.65 and 0.75 mL of said aqueous composition is transferred into the vessel(s).
55. The method of paragraph 53 or paragraph 54 wherein 0.7±0.2 mL of said aqueous composition is transferred into the vessel(s).
56. The method of any of paragraphs 51-55 wherein the method further comprises plugging the vessel, optionally wherein the vessel is plugged with a siliconized stopper.
57. The method of paragraph 53 wherein the vessel is of about 10 to 40 mm in diameter and carries at least one unit dose of drug or vaccine.
58. The method of paragraph 53 wherein the vessel is a vial, optionally a non-siliconized glass vial.
59. The method of paragraphs 51-58 wherein the method is semi-continuous or continuous.
60. The method of paragraphs 51-59 wherein step ii) takes place in a freezing chamber and wherein step iii) takes place in a drying chamber, optionally wherein the drying chamber comprises a cooling system attached thereto.
61. The method of paragraph 60 wherein the freezing chamber and the drying chamber are separated by an intermediate compartment
62. The method of paragraph 60 or paragraph 61 wherein a robotic arm transfers vessels from the freezing chamber to the drying chamber, optionally via the intermediate compartment.
63. The method of paragraphs 51-62 wherein during step i) the surface area to volume ratio is increased by greater than 1.5 times, greater than 2 times, greater than 3 times or greater than 4 times compared to the surface to area volume prior to step i).
64. The method of any of paragraphs 51-63 wherein increasing the surface area to volume ratio of the aqueous composition is achieved by rotating a vessel containing the aqueous composition at a speed not less than that required to form and maintain the aqueous composition in a layer of substantially uniform thickness against an inner surface of a circumferential wall of the vessel.
65. The method of paragraph 64 wherein the vessel containing the aqueous composition is rotated about its longitudinal axis.
66. The method of paragraphs 64 or 65 wherein the vessel containing the aqueous composition is rotated about its longitudinal axis whilst supported in a substantially horizontal orientation.
67. The method of any of paragraphs 64-66 wherein the vessel containing the aqueous composition is rotated at a speed of between 250 and 4000 rpm, between 500 and 4000 rpm, between 1000 and 4000 rpm, between 1500 and 3750 rpm, between 2000 and 4000, between 2000 and 3750 rpm, between 2000 and 3500 rpm, between 2500 and 4000 rpm, between 2500 and 3500 rpm, between 3000 and 4000 rpm, between 3000 and 3500 rpm or between 3100 and 3300 rpm.
68. The method of any of paragraphs 64-67 wherein the vessel containing the aqueous composition is rotated at a speed of between 2500 and 4000 rpm, optionally between 3000 and 3500 rpm, optionally at 3200 rpm.
69. The method of paragraphs 51-68 wherein during step ii) freezing is achieved by contacting a
vessel containing the aqueous composition with an inert cooling gas. 70. The method of paragraph 69 wherein said inert cooling gas is at a temperature of −5 to −160° C.
71. The method of paragraph 69 or paragraph 70 wherein the gas is nitrogen gas
72. The method of paragraphs 51-71 wherein during step ii) the aqueous composition is frozen at a rate of between 0.1 and 200° C./min, 0.5 and 175° C./min, 1 and 150° C./min, 2 and 150° C./min, 10 and 125° C./min or 20 and 105° C./min.
73. The method of paragraph 72 wherein the composition is frozen at a rate of between 5° and 150° C./min.
74. The method of paragraph 72 or paragraph 73 wherein the composition is frozen at a rate of between 75 and 125° C./min, optionally 100° C./min.
75. The method of paragraphs 51-74 wherein the aqueous composition is frozen in step ii) until a final vessel temperature below the critical temperature of the aqueous composition is reached.
76. The method of paragraphs 51-75 wherein the aqueous composition is frozen in step ii) until a final vessel temperature of between −4 and −200° C., between −10 and −175° C., between −30 and −150° C., between −30 and −100° C., between −30 and −80° C., between −40 and −100° C., between −60 and −100° C. or between −60 and −80° C. is reached.
77. The method of paragraph 76 wherein the aqueous composition is frozen until a final vessel temperature of −70° C. is reached.
78. The method of paragraphs 51-77 wherein the aqueous composition is rapidly snap frozen in step ii).
79. The method of paragraph 78 wherein the aqueous composition is rapidly snap frozen in step ii) for a duration of between 10 and 300 seconds, 10 and 240 seconds, 20 and 180 seconds, 20 and 140 seconds, and 120 seconds, 20 and 100 seconds or 30 and 90 seconds.
80. The method of paragraph 78 or paragraph 79 wherein the aqueous composition is frozen for a duration of between 30 and 60 seconds.
81. The method of paragraphs 51-80 wherein during step iii) the frozen composition is subjected to a primary drying step, and after the primary drying step, is subjected to secondary drying step, both primary and secondary drying steps taking place within a drying chamber.
82. The method of paragraph 81 wherein the primary drying step is immediately followed by the secondary drying step.
83. The method of paragraphs 81 or paragraph 82 wherein the primary drying step comprises adjusting the temperature of the frozen composition to a primary drying temperature under a vacuum.
84. The method of paragraph 83 wherein the primary drying temperature is below the collapse temperature of the frozen composition.
85. The method of paragraph 83 and 84 wherein the primary drying temperature is both below the collapse temperature and the Tg′ of the frozen composition.
86. The method of paragraphs 83-85 wherein the primary drying temperature is less than 20%, less than 10% or less than 5% lower than the collapse temperature of the frozen composition.
87. The method of paragraphs 83-86 wherein the primary drying temperature is between −20° C. and −60° C., −25° C. —and −45° C. or −30° C. and −40° C.
88. The method of paragraph 87 wherein the primary drying temperature is between −28° C. and −32° C.
89. The method of paragraphs 83-88 wherein the primary drying temperature is achieved from surrounding heat energy only.
90. The method of paragraph 83 wherein the vacuum is at a pressure of between 10 and 150 μbar e.g. between 10 and 100 μbar.
91. The method of paragraph 90 wherein the vacuum is at a pressure of 80 μbar.
92. The method of paragraphs 81-91 wherein the primary drying step takes between 20 mins and 180 mins, between 30 mins and 160 mins or between 60 mins and 100 mins.
93. The method of paragraph 81 or paragraph 82 wherein the secondary drying step comprises adjusting the temperature of the composition to a secondary drying temperature under vacuum.
94. The method of paragraph 93 wherein the secondary drying temperature is above 0° C.
95. The method of paragraph 93 or paragraph 94 wherein the secondary drying temperature is between 5° C. and 50° C., 10° C. and 45° C. or 20 and 40° C.
96. The method of paragraphs 93-95 wherein the secondary drying temperature is between 32° C. and 38° C., optionally 35° C.
97. The method of paragraphs 93-96 wherein the secondary drying temperature is achieved by heat energy provided by microwaves, a heating element(s) or an infrared radiator(s).
98. The method of paragraph 97 wherein the heat energy is provided by an infrared radiator(s)
99. The method of paragraph 93 wherein the vacuum is at a pressure of 10-150 p bar e.g. 10-100 p bar.
100. The method of paragraph 99 wherein the vacuum is at a pressure of 80 p bar.
101. The method of paragraph 81 or 82 wherein the secondary drying step takes between 30 mins and 180 mins, between 30 mins and 120 mins or between 60 mins and 100 mins.
102. The method of paragraph 81 or 82 wherein the primary and secondary drying steps combined take less than 24 hours, less than 22 hours, less than 20 hours, less than 18 hours, less than 16 hours, less than 14 hours, less than 12 hours, less than 10 hours, less than 8 hours, less than 6 hours, or less than 4 hours.
103. The method of paragraphs 51-102 wherein during step ii) and iii) the temperature of the composition or of the vessel comprising the composition is measured using a sensor.
104. The method of paragraph 103 wherein the sensor is an infrared camera.
105. A freeze-dried composition obtained from the method of paragraphs 51-104.
106. The use of the pharmaceutical composition of paragraphs 1-45, the vaccine of paragraph 46 or the kit of paragraphs 48-50 in the manufacture of a medicament for treating a subject in need thereof.
107. The use of the pharmaceutical composition of paragraphs 1-45, the vaccine of paragraph 46 or the kit of paragraphs 48-50 in the manufacture of a medicament for prophylaxis in a subject in need thereof.
108. A method for eliciting an immune response in a subject in need thereof comprising administering the pharmaceutical composition of paragraphs 1-45 or the vaccine of paragraph 46 to the subject, optionally wherein the subject is a human subject.
109. The pharmaceutical composition of paragraphs 1-45 or the vaccine of paragraph 46 for use in medicine.
110. The pharmaceutical composition of paragraphs 1-45 or the vaccine of paragraph 46 for use in the treatment or prevention of disease in a subject, optionally wherein the subject is a human subject.

The invention is further illustrated by the following non-limiting examples.

### EXAMPLES

### Example 1

The goal of the experiment was to evaluate the impact of spin-freeze-drying concept-related stresses on the lipid nanoparticle (LNP)-vesicle and the mRNA encapsulated by the nanoparticle.

The mRNA construct utilised was a self-amplifying mRNA (SAM) construct encoding the human cytomegalovirus (HCMV) glycoprotein B (gB) protein. The SAM-gB construct utilised corresponds to SEQ ID NO: 11 (10,025 nucleotides)

The SAM-LNP drug substance (DS) was formulated in an aqueous mixture including the excipients 20 mM Tris (pH 8)—7.5% sucrose (m/V) and 5 mM NaCl at a final SAM concentration of 60 μg/ml. After formulation, 3 mL 2R siliconized (Müller+Müller, Holzminden, Germany) vials were filled with 0.7 mL±0.05 of the aqueous mixture and subsequently exposed to rotation (shear), freeze-thawing, and dehydration stress.

Instantly after formulating the SAM-LNP drug product, a subsample was aliquoted into a sterile microfuge tube and stored at 4° C. until further use. This sample refers to the non-stressed sample or negative control.

### Rotation Experiment

A 2R lyophilisation vial filled with SAM-LNP solution was subjected to an axial rotation alike the spinning operation in the continuous spin-freeze-drying concept. A ZRZ-2041 overhead stirrer (Heidolph, Schwabach, Germany) was fitted with a custom-made clamp that fits on the 2R vial neck. A vial was clamped and rotated along its longitudinal axis at a speed of 3200 rpm. After 10 minutes of rotation, vials were stoppered with a Helvoet FM460 bromobutyl stopper and stored at 4° C. until further use.

### Spin-Freezing

Three freezing and cooling rates were tested using identical rotation conditions. Vials were rotated in the same setup as described above at 3200 rpm. The cooling rates were employed by flowing refrigerated gas over the sides of the vial. Accurate control of the cooling rates could be accomplished by regulating the gas flow rate by monitoring the product temperature using an infrared camera (FLIR A655sc, Thermal focus, Ravels, Belgium) and the gas temperature using a gauge type-K thermocouple (Labfacility, Leeds, United Kingdom). The slow, medium, and high cooling rates are referring to 5° C./min, 50° C./min and 100° C./min and took respectively 18 min, 108 sec and 54 sec to reach a final temperature of −70° C. The spin-frozen vials were gently thawed and stored at 4° C. until further use.

### Spin-Freeze-Drying Experiment (SVU)

Formulations in the vial were gas spin-frozen with the same three cooling rates (5, 50 and 100° C./min) as described in the spin-freezing section and were subsequently dried under identical conditions. Primary and secondary drying were performed in a single-vial spin-freeze-dryer (SVU) (RheaVita, Zwijnaarde, Belgium). Throughout the entire drying process, a vacuum of 30 μbar was employed and the vials were rotated at 5 rpm to avoid local overheating. Radiative heating from the drying chamber was used for sublimation and desorption. Thermography (-infra-red camera) was used to monitor the vial temperature during the drying process. At the end of the drying process, the drying chamber was vented with air to atmospheric pressure and the vial was stoppered using the mechanical stoppering system. All vials were sealed by crimping an aluminum cap over the rubber stopper and neck of the vial. The lyophilised vials were stored at 4° C. until further analysis.

### Characterization

The loss in SAM (I) encapsulation, (II) content and change in (III) hydrodynamic diameter (Z-average) and (IV) mono dispersity (PDI) of the nanoparticle was measured to evaluate deterioration due the spin-freeze-dry related stresses. Prior to the analysis, lyophilizates were reconstituted using the same volume of nuclease-free water as before drying (643 μL), without correction of the residual moisture content still present in the cake.

Dynamic light scattering (DLS): The polydispersity index (PDI) and hydrodynamic diameter (Z-average) of the nanoparticle was measured by dynamic light scattering (DLS) using a Zetasizer Nano ZS (Malvern Panalytical Ltd., Worcestershire, UK). Samples were diluted to a final concentration of 1.5 μg SAM/ml using 20 mM Tris+5 mM NaCl (pH 8). 120 μL of the diluted sample was then transferred to a ZEN0040 Disposable micro cuvette and analyzed with a measurement angle of 173° backscatter (NIBS default) a measurement temperature of 25° C. and with automatic measurement duration. A refractive index of 1.341 and a viscosity of 1.1147 kg/(m*sec) were assumed for the diluted sample. Each sample was analyzed in triplicate.

Ribogreen assay: A Quant-it RiboGreen RNA Assay Kit (Invitrogen, Merelbeke, Belgium) was used to evaluate the encapsulation of SAM by the nanoparticle. Ribogreen® reagent is an ultra-sensitive fluorescent nucleic acid stain for quantifying mRNA in solution. Vesicle encapsulation efficiency was determined by comparing the mRNA content in the absence and presence of 1% (w/v) Triton detergent. The detergent lysis the LNP lipids and makes the mRNA accessible for the RiboGreen dye. In the absence of a detergent, the RiboGreen dye can only bind on the free (not encapsulated) mRNA. The reconstituted vaccine was first diluted to a final concentration of 1.5 μg/mL using 20 mM Tris and 5 mM NaCl. 50 μL of the diluted sample was transferred to a black 96 well plate either containing 50 μL of TE buffer or 50 μL if 1% Triton- (Sigma Aldrich, Zwijnaarde, Belgium). Next, 100 μL of 1:100 diluted Ribogreen reagent was added to each well. After 15 minutes of incubation at 37° C., the fluorescence intensity was measured using a fluorescence plate reader. Quantification of the mRNA was done by comparing the fluorescence intensity of the samples against a 6-point calibration curve either in the presence of TE or Triton buffer.

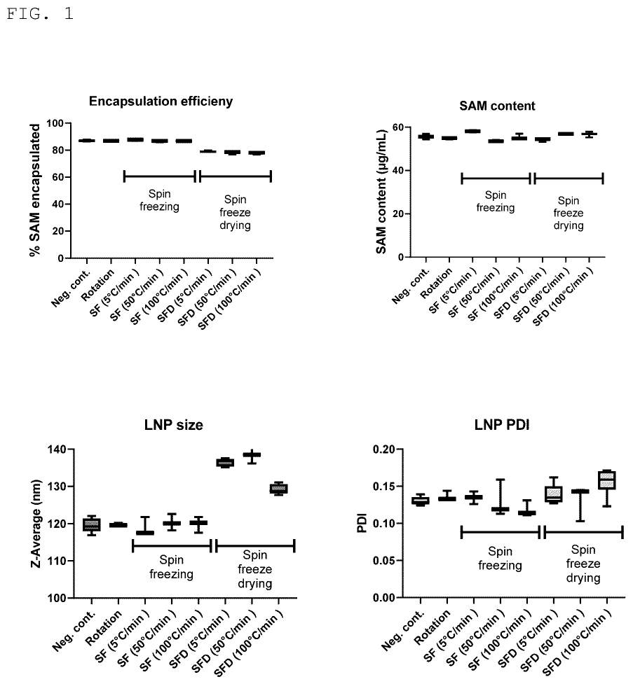

The results of this experiment are summarized in the Table 1 and shown in FIG. 1:

TABLE 1
Summary of CQA results of spin-freeze-dry related-stresses.

| | Cooling | Ribogreen | Ribogreen | % SAM loss | DLS - | | |
|---|---|---|---|---|---|---|---|
| | and | (average | (average | release | Z-average | % | |
| | freezing | values) - SAM | values) - SAM | compared | (nm) | Increase | DLS - |
| | rates | content | encapsulation | pre-lyo | mean - | in LNP | LNP |
| | (° C./min) | (μg/mL) | (%) | (%) | LNP size | Size | PDI |
| Ctrl - aliquot | NaN | 55.5 | 87 | 0 | 120 | 0 | 0.13 |
| start material | | | | | | | |
| Rotation | NaN | 54.9 | 87 | 0 | 120 | 0 | 0.137 |
| experiment | | | | | | | |
| Spin-freezing | 5 | 58.1 | 87 | 0 | 119 | −1 | 0.135 |
| | 50 | 53.7 | 86 | −1 | 120 | 0 | 0.13 |
| | 100 | 55.5 | 87 | 0 | 120 | 0 | 0.119 |
| Spin- | 5 | 54.4 | 79 | −9 | 137 | 14 | 0.139 |
| freeze-drying | 50 | 56.9 | 78 | −10 | 139 | 16 | 0.13 |
| | 100 | 56.8 | 78 | −10 | 129 | 8 | 0.156 |

### Conclusion

The size, polydispersity index (PDI) and encapsulation efficiency of LNPs didn't change after spin-freezing and thawing, indicating that the concept-related freezing step didn't change the basic properties of the SAM-LNP vaccine. After dehydration a slight change in nanoparticle size and SAM encapsulation was observed.

The lyophilizates had a uniform appearance without any minor or major defects. Reconstitution was achieved in less than 10 seconds without additional swirling. The reconstitutes were uniform and translucent just like the freshly formulated SAM-LNP solution.

### Example 2

The purpose of this experiment was to identify the required secondary drying temperature to achieve a similar residual moisture content as for conventional freeze-dried samples, which target 1% w/w.

A continuous freeze-drying engineering prototype (RheaVita, Zwijnaarde, Belgium) produced three batches of lyophilizates with varying residual moisture content by heating the product to 22° C., 30° C. and 35° C. using infra-red radiation during secondary drying. Encapsulation efficiency and nanoparticle morphology were determined on reconstituted samples to evaluate the elevated temperatures.

SAM-LNPs were formulated in an aqueous mixture comprising the excipients 20 mM Tris (pH 8) −7.5% sucrose W/V and 5 mM NaCl. After the formulation step, 3 mL siliconized (Müller+Müller, Holzminden, Germany) were filled with 0.7 mL±0.05 of the aqueous mixture and stored at 4° C. until the experiments. An aliquot of the freshly prepared medium was frozen and stored in an ultralow freezer (−80° C.) as a benchmark sample (negative control).

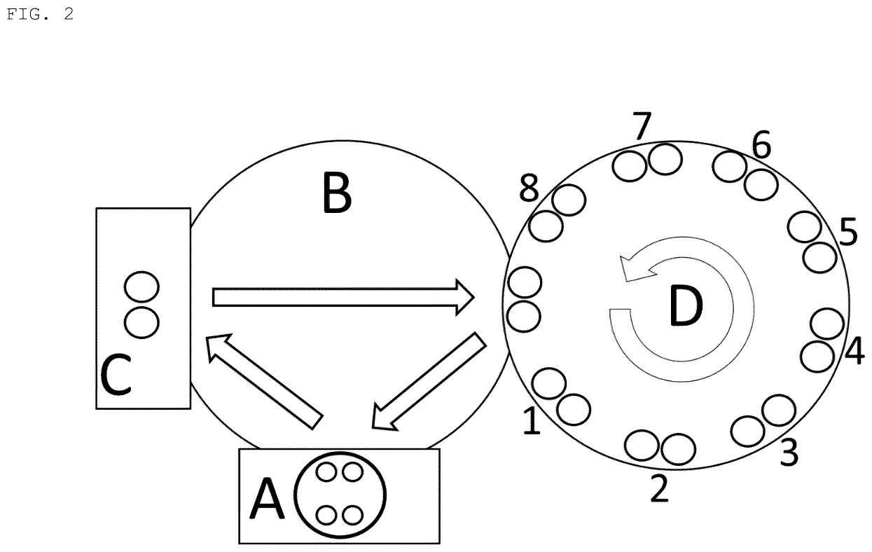

FIG. 2 represents a schematic overview of the equipment and explains the index position (indicated with numbers in the drying chamber (D)).

### Spin-Freezing

Vials were rotated at 3200 rpm to spread the aqueous drug formation over the inner surface of the vial, whilst a cold air flow is used for cooling and freezing of the aqueous solution at a rate of 100° C./min. After ice crystallization, vials were further cooled until −70° C. for complete solidification and were then transferred by a robotic arm to the drying chamber of the continuous freeze-dryer.

### Drying

The total duration of the drying process was fixed at 150 minutes, which included 60 and 90 minutes for the primary and secondary drying, respectively.

A vacuum of 80 μbar was controlled by a Pirani manometer for both primary and secondary drying.

Primary drying took place in the first three index positions (FIG. 2). Radiative heating emitted by the drying chamber drove the sublimation, without the mediation of the infra-red radiators (No possibility to monitor the wall temperature of the drying chamber).

Secondary drying started when vials moved from index position three to four. At this index position the vial temperature was ramped to the target vial temperature. After 17 minutes the vial moved to the next position and from this point until the end of the process, the vial was fixed at the desired vial temperature (22, 30 or 35° C.).

Throughout the entire secondary drying, a FLIR infrared camera was used to monitor the vial temperature and to adjust the infra-red power supply to achieve and maintain the desired vial temperature.

After the secondary drying, vials were removed from the drying chamber by a robotic arm and placed in a glovebox filled with dry nitrogen at atmospheric pressure. Vials were stoppered with a Helvoet FM460 bromobutyl stopper and sealed by crimping an aluminum cap over the rubber stopper and neck of the vial.

Table 2 summarizes the electrical power required to achieve the target vial temperature per index position in the drying chamber:

TABLE 2
Power provided by IR heater to spin frozen vial per index position
to reach the desired vial temperature during secondary drying.

| | Time spent per | Power Infrared Radiator (W) | | |
|---|---|---|---|---|
| Index | index position (mins) | 22° C. | 30° C. | 35° C. |
| 1 | 17 | 0 | 0 | 0 |
| 2 | 17 | 0 | 0 | 0 |
| 3 | 17 | 0 | 0 | 0 |
| 4 | 17 | 0 | 0.3 | 0.3 |
| 5 | 17 | 0 | 0.6 | 0.6 |
| 6 | 17 | 0 | 1.2 | 1.2 |
| 7 | 17 | 0 | 0.8 | 1.2 |
| 8 | 17 | 0 | 0.8 | 1.2 |
| 9 | 8.5 | 0 | 0 | 0 |

### Karl Fisher Moisture Content

The residual moisture content of the freeze-dried cakes was determined using a Karl-Fisher titrator (831 KF Coulometer, Metrohm Belgium) connected to 774 Oven Sample Processor (Metrohm AG, Herisau, Switzerland). 7 mL capped vials (Metrohm Belgium), containing 20-25 mg crushed freeze-dried cake were placed in the oven to transfer the water as a vapor to a titration vessel with an anolyte solution (Hydranal coulomat A for oven, Sigma-Aldrich Belgium). The moisture content was calculated from a standard plot (water standard oven 1%, Apura, Merck), based on the sample weight and the amount of water evaporated into the titration vessel. Three blank vials were analyzed to subtract the background moisture from the freeze-dried cake samples. All sample manipulations were performed in a nitrogen glove box (Jacomex, Belgium) with relative humidity and oxygen conditions below 1 ppm.

### Dynamic Light Scattering and Encapsulation

DLS and Ribogreen were performed as described in Example 1 above

### Capillary Gel Electrophoresis:

The relative integrity of the mRNA comprised in the lyophilised composition was determined using capillary gel electrophoresis after the mRNA was extracted with chloroform from the with Triton-X disrupted LNP's.

### Results

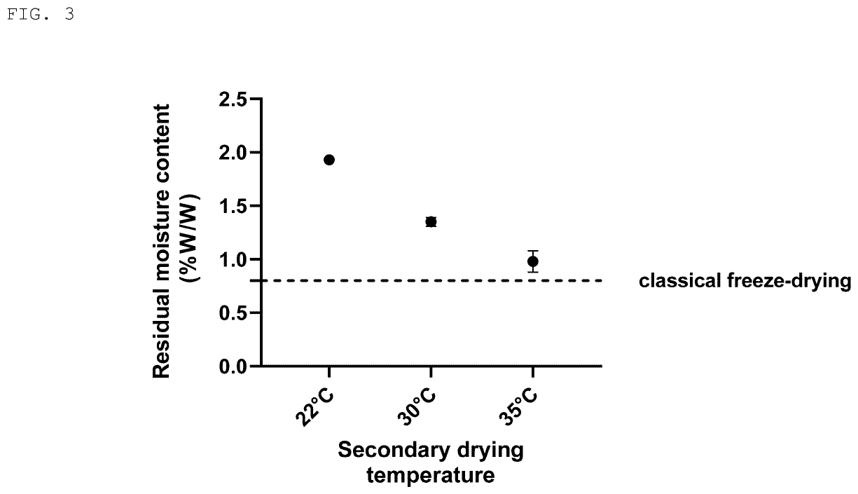

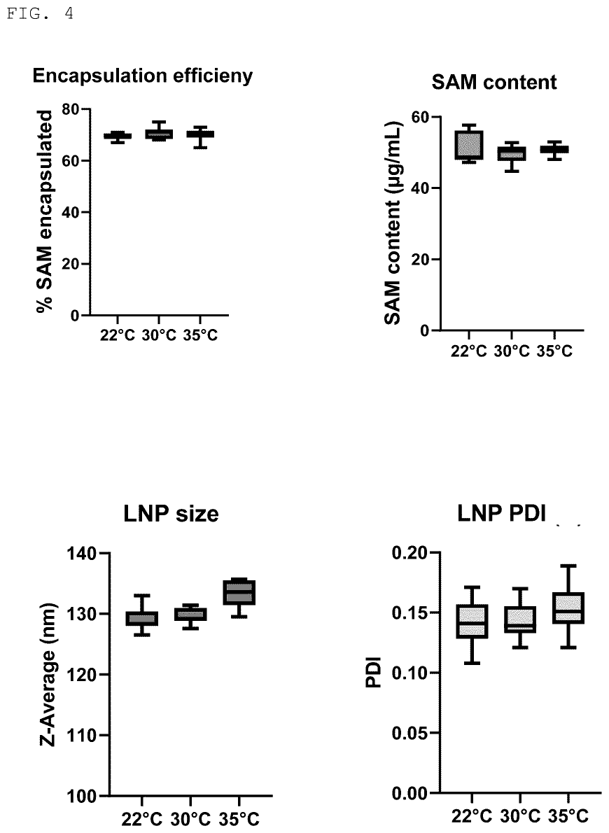

The results of this experiments are presented in Table 3 below and illustrated in FIGS. 3 and 4

TABLE 3
Summary of CQA results when dried at different secondary drying temperatures.

| | | Ribogreen | Ribogreen | % SAM loss | DLS - | | |
|---|---|---|---|---|---|---|---|
| Secondary | Residual | (average | (average | release | Z-average | % | |
| drying - vial | moisture | values) - SAM | values) - SAM | compared | mean - | Increase | DLS - |
| temperature | content | content | encapsulation | pre-lyo | LNP size | in LNP- | LNP |
| (° C.) | (% W/W) | (μg/mL) | (%) | (%) | (nm) | size (%) | PDI |
| Ctrl - aliquot | NaN | 50.4 | 80 | 0 | 118 | 0 | 0.144 |
| start material | | | | | | | |
| 22° C. | 1.68 | 51.1 | 69 | −14 | 129 | 9 | 0.141 |
| 30° C. | 1.35 | 49.6 | 71 | −11 | 130 | 10 | 0.143 |
| 35° C. | 0.98 | 50.8 | 70 | −13 | 133 | 12 | 0.153 |

### Conclusion

Dehydration reduces encapsulation efficiency by about 10% while increasing the hydrodynamic diameter of the nanoparticles by about 10 nm when compared to a freshly prepared SAM-LNP solution. According to the data in table 3, increasing secondary drying does not lead to higher encapsulation losses or an increase in nanoparticle size. Moreover, the various secondary drying temperatures had no effect on the spin-freeze-dried cake appearance and the rehydration behavior. The produced cakes were uniform and elegant, showing no signs of cracks, shrinkage, or collapse. Reconstitution was achieved in less than seconds without additional swirling. The reconstitutes were uniform and translucent just like the freshly formulated SAM-LNP solution.

However, the target residual moisture content (<1%) was only achieved when the vial temperature was maintained for 90 minutes at 35° C. (residual moisture content=0.98% W/W). Lower secondary drying temperatures resulted in cakes with residual moisture contents above the target value of 1% W/W.

### Example 3

The goal of this experiment was to compare conventional freeze-drying to spin-freeze-drying using the optimized conditions as described in examples 1 and 2 hereinabove. SAM-LNPs were formulated in a solution containing the excipients 20 mM Tris (pH 8)—7.5% sucrose (m/V) and 5 mM NaCl. 3 mL 2R siliconized (Muller and Muller) vials were filled with 0.7 mL±0.05 of the aqueous mixture and divided into two halves. The concentration of SAM in the formulation was as described in Example 1 (i.e. 60 μg/ml). One-half of the prepared vials was intended for spin-freeze-drying, while the other half was intended for conventional freeze-drying. An aliquot of the freshly prepared solution was frozen at −80° C. as a negative control.

Vials destined for conventional freeze-drying were semi-stoppered (partially inserted to allow water vapor to escape during the freeze-drying cycle) and transferred into the freeze-dryer and were subjected to the freeze-drying cycle and composed to the following steps:
1. Freezing
   a. Samples were loaded at room temperature on shelves of the freeze-dryer and were thermally equilibrated at 5° C. for 30 minutes, followed by 30 minutes at −5° C. The applied cooling rates between the loading and equilibration phases were 1° C./min.
   b. Freezing was induced by cooling the vial at 1° C./min to a final temperature of −40° C.
2. Primary drying
   a. The chamber pressure was set at 57 mTorr (or 76 μbar) and the shelf temperature was raised from −40° C. to −29° C. over a period of 22 minutes. The chamber pressure and the shelf temperature were maintained for 27 hours.
3. Secondary drying
   a. The shelf temperature was raised from −29° C. to 15° C. over a period of 440 minutes. The shelf temperature and chamber pressure 57 mTorr (or 76 μbar) were maintained for 12 hours

This batch-freeze drying protocol is also summarized in the table below:

| | Temperature | Time | Vacuum | Ramp |
|---|---|---|---|---|
| Cycle Step | (° C.) | (hours) | (mTorr) | Rate |
| Sample Loading | RT | — | — | — |
| Thermal | 5 | 0.5 | — | 1° C./min |
| Equilibration | −5 | 0.5 | — | 1° C./min |
| Freezing | −40 | 1 | — | 1° C./min |
| Primary Drying | −29 | 27 | 57 | 0.5° C./min |
| Secondary Drying | 15 | 12 | 57 | 0.1° C./min |

At the end of the conventional freeze-drying cycle, the drying chamber was backfilled with dry nitrogen until a 900 mbar chamber pressure was reached. Semi-stoppered vials were stoppered and unloaded directly after the end of the secondary drying. Vials were sealed with aluminum flip off caps and stored at −20° C. until further analysis.

The second half of the filled vials were spin-frozen and dried using the continuous freeze-drying engineering prototype (FIG. 2) under the following conditions:
Spin-freezing
   Vials were rotated at 3200 rpm to spread the aqueous drug formulation over the inner surface of the vial, whilst a cold gas flow is used for cooling and freezing the aqueous solution at a rate of 100° C./min. After ice crystallization, vials were further cooled until −70° C. and were then transferred by a robotic arm to the drying chamber of the continuous freeze-dryer.
Drying
   The total time of the drying cycle was fixed at 180 minutes of which 90 minutes primary drying and 90 minutes secondary drying
   A chamber pressure of 80 μbar was controlled by a Pirani manometer for both the primary and secondary drying.
   Primary drying took place in the first four index positions. Radiative heating emitted by the pre-cooled drying chamber was used for sublimation without the aid of the infra-red radiators.
   Secondary drying started when vials moved from index position 4 to 5. At this index position the vial temperature was ramped to the target vial temperature. After 21 minutes the vial moved to the next position and from this point until the end of the process, the vial temperature was maintained at 35° C.
   Throughout the entire secondary drying, a FLIR infrared camera was used to monitor the vial temperature and to adjust the infra-red power supply to achieve and maintain the desired vial temperature.
   After the secondary drying, vials were removed from the drying chamber by a robotic arm and placed in a glovebox filled with dry nitrogen at atmospheric pressure. Vials were stoppered with a Helvoet FM460 bromobutyl stopper and sealed by crimping an aluminum cap over the rubber stopper and neck of the vial.

Table 4 summarizes the electrical power required to achieve the target vial temperature per index position in the drying chamber:

TABLE 4
Power provided by IR heater to spin frozen vial per index position
to reach the desired vial temperature during secondary drying.

| | Time spent per index | Power infrared radiator (W) |
|---|---|---|
| Index | position (mins) | 35° C. |
| 1 | 21 | 0 |
| 2 | 21 | 0 |
| 3 | 21 | 0 |
| 4 | 21 | 0 |
| 5 | 21 | 0.3 |
| 6 | 21 | 0.6 |
| 7 | 21 | 1.2 |
| 8 | 21 | 1.2 |
| 9 | 11 | 0 |

Lyophilizates were stored at −20° C. until further analysis.

Characterization of the lyophilizates (e.g. Karl fisher, DLS, Ribogreen, capillary gel electrophoresis etc.) was conducted as described in Example 1 and Example 2 above.

### Results

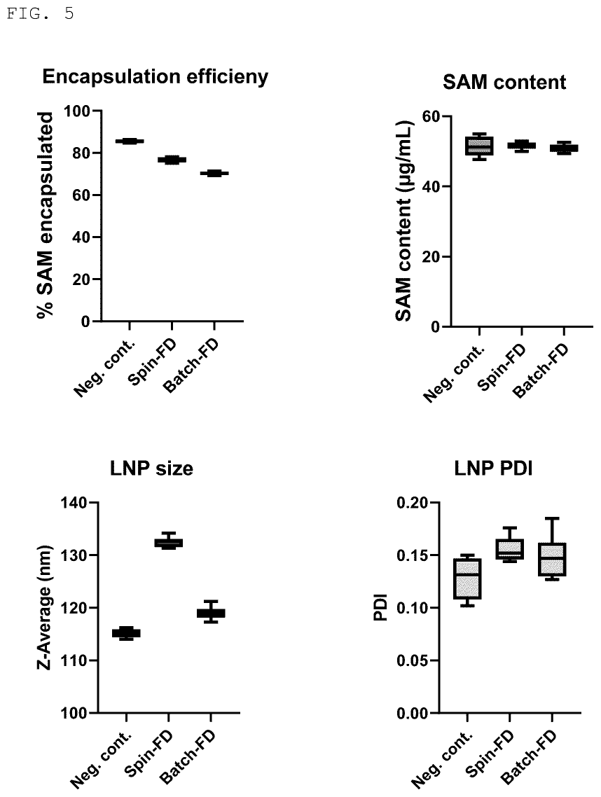

The results of this experiment are presented in the table 5 and illustrated in FIG. 5

TABLE 5
Summary of CQA results when dried at different secondary drying temperatures.

| | | Ribogreen | Ribogreen | % SAM loss | DLS - | % Increase | |
|---|---|---|---|---|---|---|---|
| | Residual | (average | (average | release | Z-average | in LNP- | |
| Secondary | moisture | values) - SAM | values) - SAM | compared | (nm) | size (z- | DLS - |
| drying - vial | content | content | encapsulation | pre-lyo | mean - | average) | LNP |
| temperature | (% W/W) | (μg/mL) | (%) | (%) | LNP size | (%) | PDI |
| Ctrl - aliquot | NaN | 51.4 | 86 | 0 | 115 | 0 | 0.129 |
| start material | | | | | | | |
| Spin- | 1.14 | 51.7 | 77 | −10 | 132 | 15 | 0.155 |
| freeze-drying | | | | | | | |
| Freeze-drying | 1.15 | 51 | 70 | −19 | 119 | 3 | 0.149 |

As can be seen from the data in Table 5 and FIG. 5, batch freeze drying results in an encapsulation efficiency of 70% (a loss of 19% compared to pre-lyophilisation). However, spin-freeze drying resulted in an improved encapsulation efficiency of 77% (a loss of just 10% compared to pre-lyophilisation). This is surprising, especially considering the increase in LNP size observed with the spin-freeze drying protocol and the fact that the self-amplifying mRNA construct was large (>10k ribonucleotides). The spin and batch freeze-dry yielded cakes with similar residual moisture content and without minor or major defects such as, cracks, shrinkage, or collapse. Reconstitution was achieved in less than 10 seconds without additional swirling. The reconstitutes were uniform and translucent just like the freshly formulated SAM-LNP solution.

### Example 4

### Methods and Materials

Vials intended for spin-batch freeze-drying and radiative spin-freeze-drying were spin-frozen together using a standalone custom-made spin-freezing setup. 2R glass vial filled with 0.5 mL of mRNA-LNP solution was placed vertically into a WB6000-D overhead stirrer (Wiggens, Beijing, China) using a custom-made 2 ml vial adapter. The vial was rotated around its longitudinal axis at 4000 rotation per minute (rpm) and cooled by a cryogenic gas at a cooling rate of 100° C./min. When the vial temperature reached −50° C., samples was removed and placed in a −70° C. freezer until lyophilization.

Next, the spin-frozen vials were randomly divided in two batches. One batch was used for shelf spin-freeze-drying these vials were placed in pre-cooled (−70° C.) custom made vial holder. The second batch of vials was kept in the −80° C. freezer and were dried one by one by in the single vial freeze-dryer.

### Freeze-dryers

BFD: Batch freeze-drying.
   (Traditional, conventional)
   cakes are on the bottom of the vials.
SFD: shelf-spin-freeze-drying or spin-batch freeze-drying
   Vials are spin-frozen by rotating the vial at high rotation speed (4000 rpm) while blowing refrigerated gas on the side of the vials.
   After the formulation is spin-frozen and cooled to a temperature below (−40° C.) vials
   are placed in pre-cooled vial holders (FIG. 1).
SVU or MVU: Radiative-spin-freeze-drying:
   Lyophilization is carried out either in the SVU or MVU
   SVU has three configurations.
Without wall cooling
With external wall cooling: thermal radiation is reduced by cooling the walls of the drying chamber using an external chiller.
With radiative shield: a removable wall cooling is placed inside the drying chamber,

### Dynamic Light Scattering (DLS)

The size of the mRNA-LNP was determined by dynamic light scattering (DLS) as described above.

### Ribogreen

mRNA content and encapsulation was determined using a Ribogreen kit as described above.

### Karl Fisher

Residual moisture of the dried cake was determined by the above described “Karl Fischer” methods.

### Capillary Electrophoresis

The mRNA integrity was analyzed by capillary electrophoresis using an Agilent 4200 TapeStation system (Agilent Technologies, United States) and the kit.

First, the mRNA was extracted from the LNP by ethanol precipitation followed by dissolving the precipitated pellet in RNAse free water. The raw data was analyzed using the Tapestation analysis software (Agilent Technologies, United States). mRNA integrity of the mRNA-LNP samples is presented as the percentage to the mRNA drug substance mRNA before formulating in LNP's. Both, the DS and DP were assayed within the same run

In Vitro Potency by Fluorescence-Activated Cell Sorting (FACS) for Cells Expressing Enhanced Green Fluorescent Protein (eGFP)

BHK-21 cells were maintained in DMEM High Glucose medium (HyClone) complemented with 10% fetal bovine serum (Gibco/Life Tech.) and 1% penicillin/streptomycin/L-glutamin (Gibco-Invitrogen) and incubated at 37° C., 5% CO2. Twenty-four hours before transfection, cells were seeded in 96-well plates at a density of 26 666 cells per well in 150 μl DMEM High Glucose complemented with 5% FBS and 1% Glutamine (Gibco/Life Tech). The cells were transfected by incubating a serial dilution of the mRNA-LNP over a range of 1-400%. After overnight incubation, cells were harvested, washed with PBS, fixed, and permeabilized. The eGFP expression in BHK cells was detected by MACSQuant flow cytometry (Miltenyi Biotech, Bergisch Gladbach, Germany). The in vitro potency, defined as the percentage of eGFP+ cells, was analyzed via global fit of 4-PL analysis and expressed relative to an internal standard LNP formulation and to the pre-lyophilized product.

### SAM-RNA LNP

The mRNA construct utilized was a self-amplifying mRNA (SAM) construct encoding the human cytomegalovirus (HCMV) glycoprotein B (gB) protein. The SAM-gB construct utilized.

The SAM-LNP drug substance (DS) was formulated in an aqueous mixture including the excipients 20 mM Tris (pH 8)—7.5% sucrose (m/V) and 5 mM NaCl at a final SAM concentration of 60 g/ml. After formulation, 3 mL 2R siliconized (Müller+Müller, Holzminden, Germany) vials were filled with 0.7 mL±0.05 of the aqueous mixture and subsequently freeze-dried using a traditional batch freeze-drying, shelf-spin-freeze-drying and radiative spin-freeze-drying. Instantly after formulating the SAM-LNP drug product, a subsample was aliquoted into a sterile microfuge tube and stored at −80° C. until further use. This sample refers to the final bulk or non-stressed or negative control.

Various radiative spin-freeze-drying conditions were created by controlling both the thermal (i.e., radiation emitted by drying chamber walls) and infrared radiation (infrared heaters). Minimal radiative energy contribution was achieved by lowering the temperature of the surrounding drying chamber wall and by turning the infrared heating off. Radiative spin-freeze-drying experiments were carried out in a single vial freeze-dryer (Rheavita, zwijnaarde) equipped with a radiative shield to reduce thermal radiation and cooled using refrigerated nitrogen gas. Four runs were performed.

### Condition 1

Vial temperature was equal to the collapse temperature.
Ramp rate to secondary drying 0.5° C./min

### Condition 2

Vial temperature was equal to the collapse temperature.
Ramp rate to secondary drying 1° C./min

### Condition 3

Vial temperature was approximately 5° C. above the collapse temperature
Ramp rate to secondary drying 1° C./min

### Condition 4

Vial temperature was approximately 5° C. above the collapse temperature
Ramp rate to secondary drying 0.5° C./min

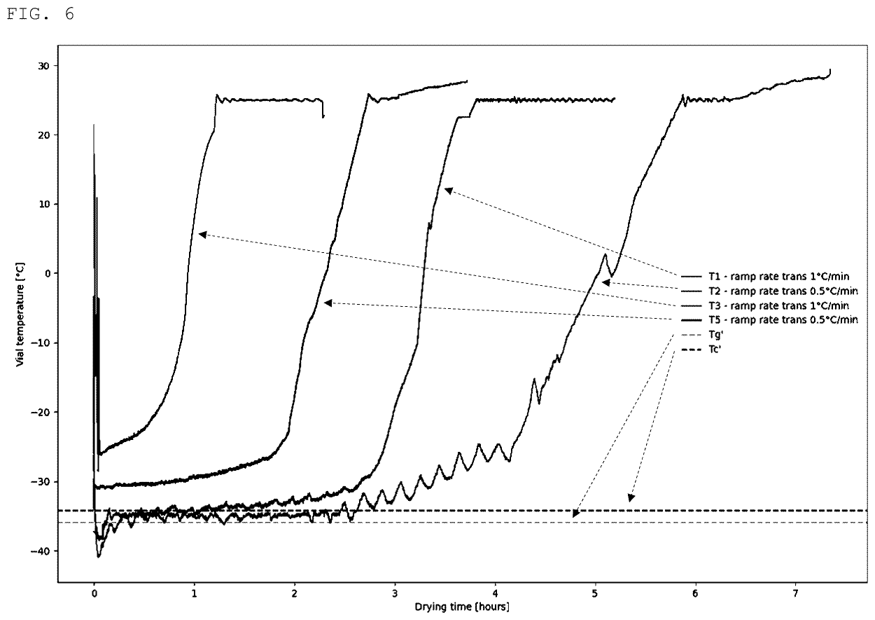

See FIG. 6 for a graphical representation of vial temperatures over drying times for Conditions 1-4. See the following table for a summary of the above conditions.

| | mRNA | | | | Fill volume | | | |
|---|---|---|---|---|---|---|---|---|
| Study 1 | construct | Vials | LNP | Stabilizers | (mL) | BFD | SFD | SVU |
| Study 1 | SAM | 2R vials | RV39- | 7.5% | 0.7 | x | x | x |
| | | non- | DMPE- | sucrose + | | | | |
| | | siliconized. | PEG- | 20 mM Tris | | | | |
| | | | Chol- | (pH 8) + | | | | |
| | | | DSPC | 5 mM NaCl | | | | |

### Radiative Spin-Freeze-Drying Conditions

Traditional batch freeze-drying and shelf spin-freeze-drying served as positive control. The impact of the ramp rate was investigate by two freeze-dry cycle whereby on the ramp rate to secondary drying was changed.
Batch-freeze-drying a slow ramp rate to secondary drying as in the table below:

| | | | Chamber | |
|---|---|---|---|---|
| | Shelf | duration | pressure | duration ramp |
| Cycle step | temperature | (hours) | (μbar) | rate (° C./min |
| Loading | | | | |
| Sample Loading | RT | — | — | — |
| Thermal | 5 | 0.5 | — | 1° C./min |
| equilibration | −5 | 0.5 | — | 1° C./min |
| Freezing | −40 | 1 | — | 1° C./min |
| Primary drying | −29 | 27 | 57 | 0.5° C./min |
| Secondary drying | 15 | 12 | 57 | 0.1° C./min |

Reference freeze-drying cycle with slow ramp rate to secondary drying

Freeze-drying cycle using a 0.5° C./min ramp rate to secondary drying as in the table below:

| | | | Chamber | |
|---|---|---|---|---|
| | Shelf | Duraton | pressure | duration ramp |
| Cycle step | temperature | (hours | (μbar) | rate (° C./min |
| Loading | | | | |
| Sample Loading | RT | — | — | — |
| Thermal | 5 | 0.5 | — | 1° C./min |
| equilibration | −5 | 0.5 | — | 1° C./min |
| Freezing | −40 | 1 | — | 1° C./min |
| Primary drying | −29 | 27 | 57 | 0.5° C./min |
| Secondary drying | 15 | 12 | 57 | 0.5° C./min |

Freeze-drying cycle with higher ramp (0.5° C./min) to secondary drying.

### Results

The loss in SAM (I) encapsulation, (II) content and change in (III) hydrodynamic diameter (Z-average) and (IV) mono dispersity (PDI) of the nanoparticle was measured to assess the impact of the product temperature during primary drying and to secondary ramp rate on the physical properties of the mRNA-LNPs. Prior to the analysis, lyophilizates were reconstituted using the same volume of nuclease-free water as before drying (643 μL) without correction of the residual moisture content still present in the cake.

All lyophilizates had a uniform appearance without any minor or major defects, even though the product temperatures during conditions 3 and 4 were above the collapse temperature of the SAM-LNP formulation. Reconstitution was achieved in less than 10 seconds without additional swirling. The reconstitutes were uniform and translucent just like the freshly formulated mRNA-LNP solution.

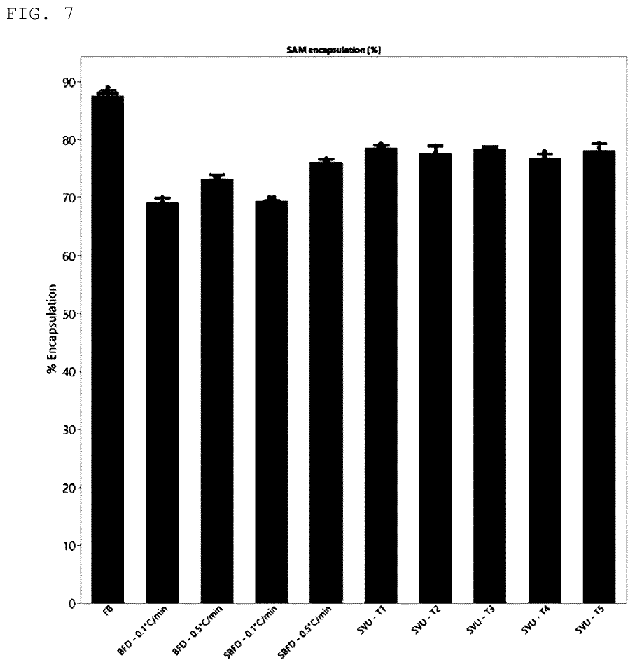

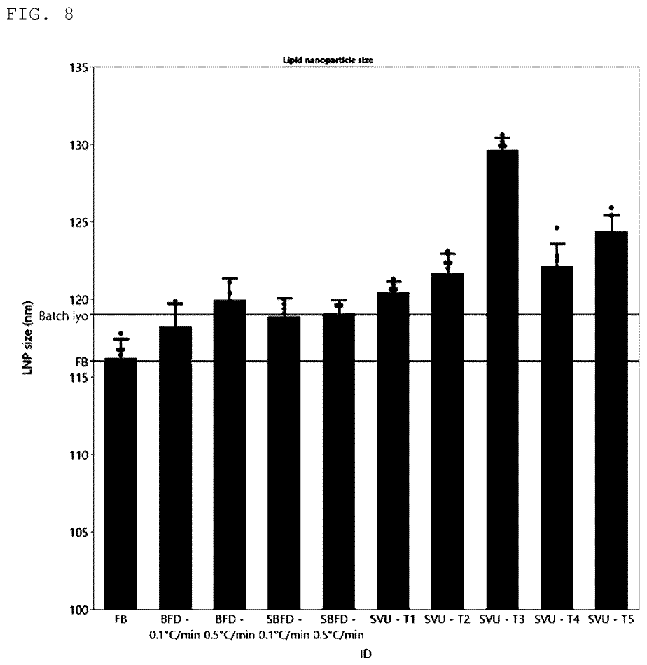

Dehydration impacted each freezing-drying protocol in measures of mRNA encapsulation (FIG. 7) and hydrodynamic diameter of the lipid nanoparticles (FIG. 8) when compared to SAM-LNP vaccines pre-lyophilization (stored at −80° C.). Nevertheless, percentage of loss in encapsulation and LNP size increase was influenced by the applied freeze-drying procedure and drying conditions. As can be seen in table and figure, higher product temperature leads to higher LNP size.

The loss in SAM encapsulation during radiative spin-freeze-drying is approximately 10% compared to a non-lyophilized SAM-LNP samples. The payload loss during radiative spin-freeze-drying was not impacted by the higher product temperature during primary drying or higher secondary drying ramp rate. On the other hand, applying higher product temperature (above the critical temperature), causes a significant increase of approx. in hydrodynamic diameter (condition T3).

### Conclusions

The lowest percentage of encapsulation was observed for the traditional (non-SVU) lyophilized samples using a ramp rate of 0.1° C./min. This freeze-dry cycle is currently used for producing reference samples.

Spin-freeze-dried (SVU) samples have higher SAM encapsulation, independent of the drying conditions used.

The ramp rate to the secondary drying plateau phase seems to be important but could not be confirmed

More aggressive SVU drying conditions cause an LNP size increase

Within SVU samples, no difference in LNP size when a ramp rate of 0.5 and 1° C./min is used.

### Example 5

### Overview of Studies 1 and 2

An accelerated stability study was conducted to determine whether the spin-lyophilized samples have the same stability as their traditional lyophilized counterparts. This accelerated stability study at 25° C. aimed primarily to discriminate between the three lyophilized formats at temperatures higher than those used to normally store lyophilization (i.e. higher than normal being at or near room temperature). mRNA-LNPs were dried using traditional (BFD), shelf-spin-freeze-drying (SFD) and radiative spin-freeze-drying (SVU). The two main differences between traditional and radiative spin freeze-drying are the way the vials are frozen, including vial rotation and product temperature ramp rates, and how heat is transferred, including comparing conduction and radiation. Shelf-spin-freeze-drying shares the spin-freezing step with SVU but heat for drying is mostly transferred via conduction with freeze-dryer shelves as opposed to via radiation as for radiative spin-freeze drying.

The mRNA constructs utilized were non-self-replicating mRNA (i.e. they were conventional mRNA) constructs encoding eGFP or nLuc.

### Spin-Freezing

Vials were rotated at 4000 rpm to spread the aqueous drug formation over the inner surface of the vial, whilst a cold air flow is used for cooling and freezing of the aqueous solution at a rate of 100° C./min. After ice crystallization, vials were further cooled to −70° C. and subsequently stored in a −80° C. freezer for either radiative or shelf-spin-freeze-drying.

### Drying

Shelf spin-freeze drying and traditional freeze drying were carried out as described above.

### Stress Testing

The thermostability of the lyophilizates was assessed by incubation the samples for 4 weeks at 25° C. Each week, samples were collected to characterize the biological activity and physical chemical properties.

The mRNA-LNP vaccines were lyophilized using batch freeze-drying (BFD), shelf-spin-freeze-drying, and spin-lyophilized using the single vial unit (SVU). The applied conditions for each of the three drying technologies are summarized in the following table:

| Accelerated | mRNA | | | Fill volume | | | |
|---|---|---|---|---|---|---|---|
| stability study | construct | LNP | Stabilizers | (mL) | BFD | SFD | SVU |
| Study 1 | eGFP-encoding | RV94- | 7.5% | 0.7 | x | x | x |
| | segment flanked | DMPE- | sucrose + | | | | |
| | by 5′ and 3′ | PEG- | 20 mM Tris | | | | |
| | UTRs when paired | Chol- | (pH 8) + | | | | |
| | called “UTR2” | DSPC | 5 mM NaCl | | | | |
| | SEQ ID NOs: 12 | | | | | | |
| | and 13, respectively. | | | | | | |
| Study 2 | eGFP-encoding | RV94- | 7.5% | 0.5 | x | x | x |
| | segment flanked | DMPE- | sucrose + | | | | |
| | by 5′ and 3′ | PEG- | 20 mM Tris | | | | |
| | UTRs when paired | Chol- | (pH 8) | | | | |
| | called “UTR3” | DSPC | | | | | |
| | SEQ ID NOs: 3 | | | | | | |
| | and 4, respectively. | | | | | | |

### Study 1

The goal of the experiment was to evaluate the impact of batch-freeze-drying, shelf-spin-freeze-drying, or radiative spin-freeze-drying with subsequent temperature stressing on the biological and physical properties of the mLRNA-LNP. The mLRNA-LNP drug substance (DS) was formulated in an aqueous mixture including the excipients 20 mMv Tris (pH 8), 7.5% sucrose (m/V), and 5 mM NaCl at a final mRNA concentration of 60 μg/ml. The mRNA construct utilized was a conventional (i.e. non-replicative) mRNA construct encoding enhanced green fluorescent protein (eGFP) or nano-luciferase (nLuc). After formulation, 3 mL 2R siliconized (Müller+Müller, Holzminden, Germany) vials were filled with 0.7 mL±0.05 of the aqueous mixture and subsequently lyophilized using the three freeze-drying procedures. An aliquot of the freshly prepared formulation was stored in an ultra-low temperature freeze and served as reference to determine the influence of both the lyophilization and temperature stress.

### Batch Freeze-Drying

Batch freeze-drying was carried out with the steps described in the following table:

| | | Shelf | Chamber | |
|---|---|---|---|---|
| | Time | temperature | pressure | Ramp rate |
| Cycle step | (hours) | (° C.) | (mbar) | (° C./min) |
| Sample loading | / | 22 | 1013 | / |
| Thermal | 0.5 | 5 | 1013 | 1 |
| equilibration | 0.5 | −5 | 1013 | 1 |
| Freezing | 21 | −50 | 1013 | 1 |
| Primary drying | 27 | −29 | 0.076 | 0.5 |
| Secondary drying | 12 | 15 | 0.076 | 0.1 |
| Stoppering | / | 15 | 900 | / |

### Spin-Freezing

2R Vials filled with 0.5 mL aqueous solutions were rotated at 4000 rpm to spread the aqueous drug formation over the inner surface of the vial, whilst a cold air flow is used for cooling and freezing of the aqueous solution at a rate of 100° C./min. After ice crystallization, vials were further cooled to −70° C. and then stored in a −80° C. freezer. Half of the samples were transferred to a pre-cooled vial holder for shelf-spin-freeze-drying. The other half was kept in the −80° C. and were processed one by one by radiative spin-freeze-drying using the single vial unit.

### Shelf Sin-Freeze-Drying

The spin-frozen vials were dried in a classical batch freeze-dryer using an aluminum custom-made vial holder. The vial holders were placed in the same −80° C. freezer as the spin-frozen vials. The spin-frozen vials were placed in the vial holders when it reached a temperature close to the temperature of the ultra-low temperature freezer and were subsequently semi-stoppered using siliconized Helvoet FM460 bromobutyl stoppers. The vial and vial holders were then placed on pre-cooled (−50° C.) shelves of a traditional batch-freeze-drying system. Next, primary and secondary drying occurred as described in the table below. At the end of the conventional freeze-drying cycle, the drying chamber was backfilled with dry nitrogen until a 900 mbar chamber pressure was reached. The vials were fully stoppered (i.e. they were semi-stoppered before) and unloaded directly after the end of the secondary drying. Vials were sealed with aluminum flip off caps and stored at −80° C. until further analysis. The following table provides details for the pressures, temperatures, time, and ramp rates of the steps described above:

| | | Shelf | Chamber | |
|---|---|---|---|---|
| | Time | temperature | pressure | Ramp rate |
| Cycle step | (hours) | (° C.) | (mbar) | (° C./min) |
| Sample loading | / | −50 | 1013 | 1 |
| Primary drying | 9 | −29 | 0.076 | 0.5 |
| Secondary drying | 5 | 15 | 0.076 | 0.1 |
| Stoppering | / | 15 | 900 | / |

### Radiative Spin-Freeze-Drying (SVU)

Radiative spin-freeze-drying (SVU) was carried out in a single vial freeze-dryer equipped with wall cooling (RheaVita, Zwijnaarde, Belgium). Two out of the six wall were cooled by circulated refrigerator oil through the double walled drying chamber, which was regulated by an external chiller. The drying chamber walls were cooled at least 30 minutes prior the drying experiments. Drying was started by placing a single vial into the vial holder and by creating a vacuum of 4 Pa. Throughout the entire drying process, a vacuum of 40 bar was employed and the vials were rotated at 5 rpm to avoid local overheating. Only thermal radiation emitted from the drying chamber wall was used for sublimation. Additional heat for desorption was provided by a separate infrared heater. The total energy input using secondary drying was set to maintain the vial temperature at 30° C. for 1 hour. Thermography (i.e. an infra-red camera) was used to monitor the vial temperature during the drying process. At the end of the drying process, the drying chamber was vented with air to atmospheric pressure, and the vial was stoppered using the mechanical stoppering system. All vials were sealed by crimping an aluminum cap over the rubber stopper and neck of the vial. The lyophilized vials were stored at 4° C. until further analysis.

### Study 1 Results

The thermostability of the lyophilizates was assessed by incubation the samples for 4 weeks at 25° C. after 0, 5, 9, 13,17, and 21 days, samples were collected and analyzed to determine the percentage of BHK in vitro expressing eGFP and change in physicochemical properties of the lyophilized mRNA-LNPs.

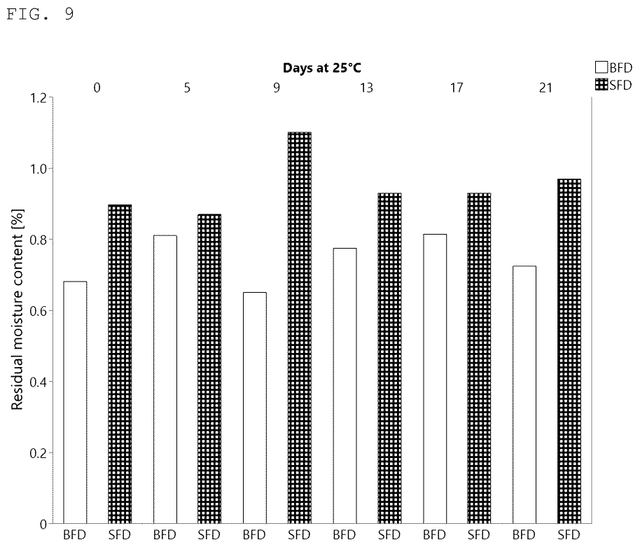

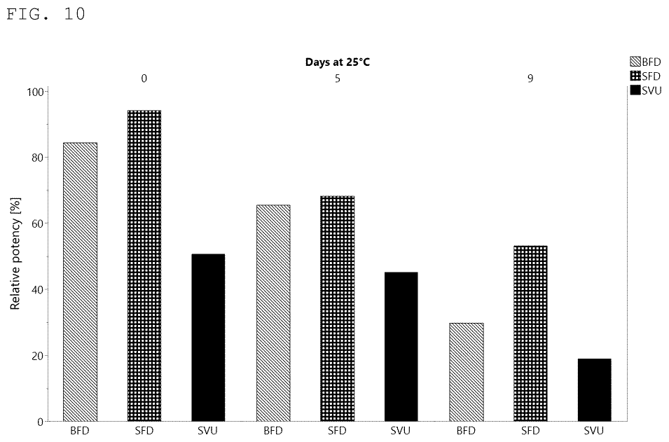

FIG. 9 shows the residual moisture content of the products over 0, 5, 9, 13, 17, and 21 days of storage at 25° C. FIG. 10 shows the percentage of baby hamster kidney (BHK) cells expressing eGFP after being transfected with the products, which had been reconstituted after 0, 5, and 9 days of storage (as lyophilized products) at 25° C. Lyophilizates were reconstituted using the same volume of nuclease-free water as before drying (643 μL), without correction of the residual moisture content still present in the cake.

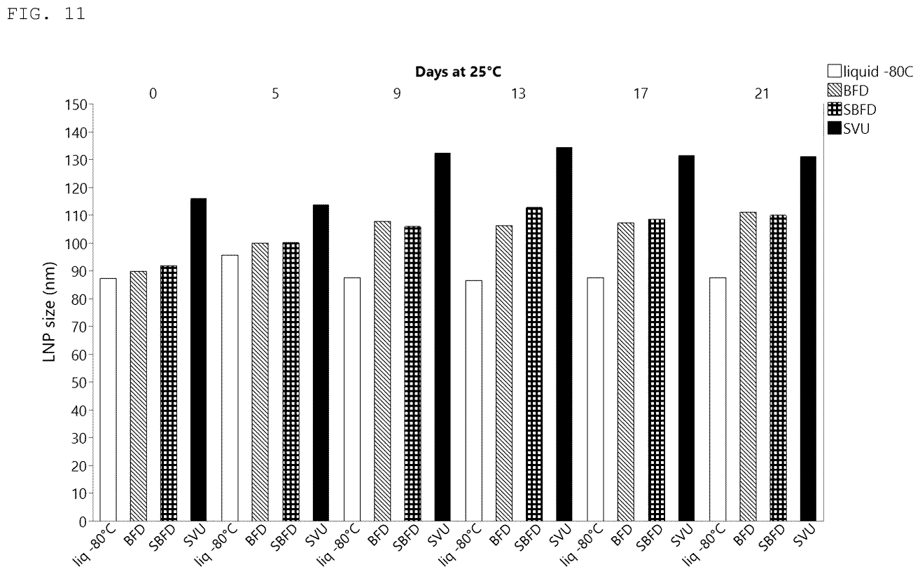

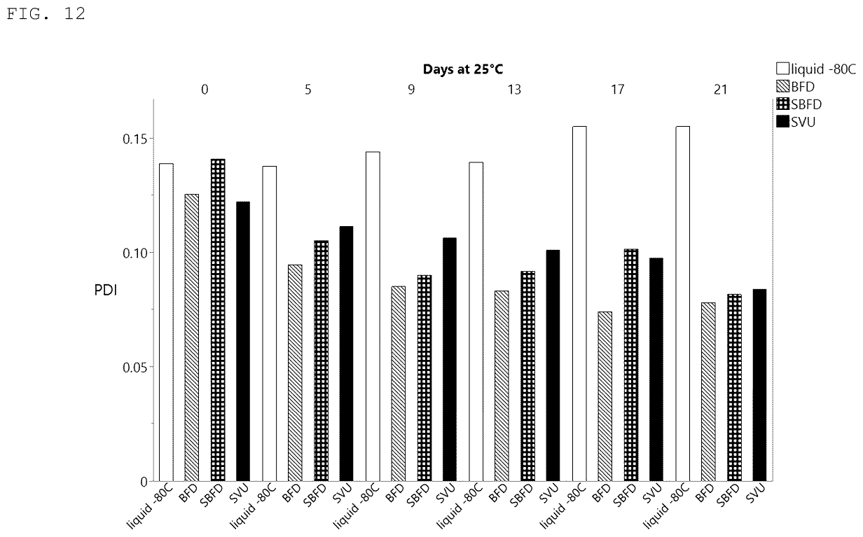

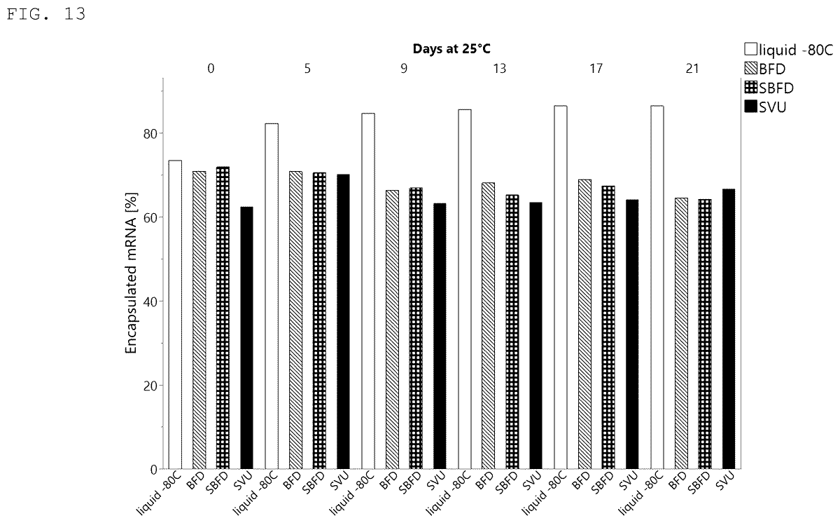

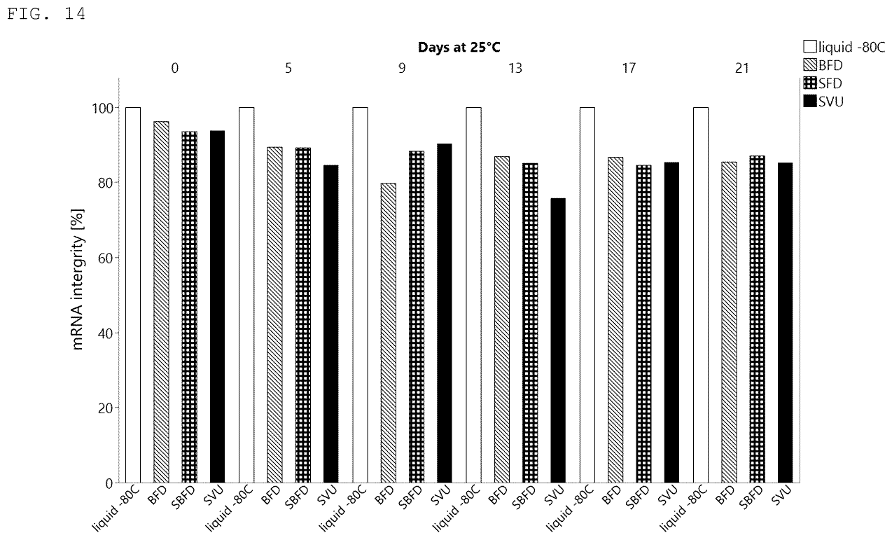

The mRNA encapsulation (FIG. 13), mRNA integrity (FIG. 14), (III) LNP Z-average hydrodynamic diameter (FIG. 11), and (IV) the LNP polydispersity index (PDI; FIG. 12) were measured to evaluate the effects of freeze-drying and stress from room temperature storage. Prior to the analysis, lyophilizates were reconstituted using the same volume of nuclease-free water as before drying (643 L), without correction of the residual moisture content still present in the cake.

All lyophilizates had a uniform appearance without any minor or major defects. Reconstitution was achieved in less than 10 seconds without additional swirling. The reconstitutes were uniform and translucent just like the freshly formulated mRNA-LNP solution.

The residual moisture content of the spin-lyophilizates dried in the single vial units were slightly higher than the target residual moisture content of 0.8%, but there was no significant difference in moisture content between the shelf lyophilized samples and batch lyophilized samples. The moisture content did not increase while the lyophilizates were incubated at 25° C. for a period of 21 days.

A significant difference exists between the single vial freeze-drying (SVU) and each of the two classical freeze-drying procedures, SFD and BED. The mRNA-LNP dehydrated by shelf spin-freeze-drying and batch-freeze-drying experience an additional reduction of approx. 10% in mRNA encapsulation when compared to an aqueous mRNA-LNP solution stored at −80° C. The radiative spin-freeze-dried shows a drop in encapsulation efficiency when compared to encapsulation before freeze-drying. Forced degradation by temperatures stressing caused a further decrease of the mRNA encapsulation when stored for 21 days at 25° C.

Dehydration negatively impacts the hydrodynamic diameter of the lyophilized nanoparticles. The sizes of the conventional (BFD) and shelf-spin-freeze-dried samples were comparable and further increase when incubated at 25° C. for 21 days. mRNA-LNP dried by radiative spin-freeze-drying experiences a further increase in size than the two other freeze-dried formats. The polydispersity index (PDI) of the lyophilizates mRNA-LNP was comparable for the three drying protocol and remained constant while temperature stressing over the 21 days.

No difference in mRNA integrity could be detected across the different drying technologies immediately after drying or when temperature stressed. The temperature stress generally caused a decline in mRNA integrity when compared to that of the formulation before freeze-drying and stored at −80° C.

The radiative spin-freeze-dried mRNA-LNP show a significant lower percentage of BHK cells expressing eGFP than the conventional batch and shelf spin-lyophilized immediately after drying. The generally lower percentages of eGFP-expressing cells across conditions was due to the selection of 5′ and 3′ UTRS, the pair called “UTR2”, which has previously been shown to result in lower expression of reporter genes (see WO2023/242817, e.g. FIG. 1). No significant difference in the percentage of cells expressing eGFP could be see between the conventional batch-freeze-dried and shelf-spin-freeze-dried vaccines.

### Study 2

The goal of this experiment was to repeat the first accelerated stability study using 5′ and 3′ UTRs which when paired are called “UTR3” in WO2023/242817. Generally, UTR3 results in higher expression of the gene of interest than the expression of the gene with UTR2. A potential impact of the secondary drying temperature was excluded by applying the same secondary drying temperature across the three drying technologies. To achieve comparable residual moisture content, the duration of this desorption phase was adjusted for each drying technology.

The mRNA-LNP drug substance (DS) was formulated in an aqueous mixture including the excipients 20 mM Tris (pH 8)—7.5% sucrose (m/V) at a final mRNA concentration of 60 μg/ml. After formulation, 3 mL 2R non-siliconized (Müller+Müller, Holzminden, Germany) vials were filled with 0.5 mL±0.05 of the aqueous mixture and subsequently lyophilized using the three freeze-drying procedures.

Batch freeze-drying (BFD)

| | | Shelf | Chamber | |
|---|---|---|---|---|
| | Time | temperature | pressure | Ramp rate |
| Cycle step | (hours) | (° C.) | (mbar) | (° C./min) |
| Sample loading | / | 22 | 1013 | / |
| Thermal | 0.5 | 5 | 1013 | 1 |
| equilibration | 0.5 | −5 | 1013 | 1 |
| Freezing | 21 | −50 | 1013 | 1 |
| Primary drying | 27 | −29 | 0.076 | 0.5 |
| Secondary drying | 12 | 25 | 0.076 | 0.1 |
| Stoppering | / | 25 | 900 | / |

### Spin-Freezing

2R Vials filled with 0.5 mL aqueous solutions were rotated at 4000 rpm to spread the aqueous drug formation over the inner surface of the vial, whilst a cold air flow is used for cooling and freezing of the aqueous solution at a rate of 100° C./min. After ice crystallization, vials were further cooled until −70° C. and subsequently stored in a −80° C. freezer for either radiative or shelf-spin-freeze-drying.

### Shelf Sin-Freeze-Drying (SFD)

The spin-frozen vials were dried in a classical batch freeze-dryer using an aluminum custom-made vial holder. The vial holders were placed in the same −80° C. freezer as the spin-frozen vials. The spin-frozen vials were placed in the vial holders when it reached a temperature closed to the temperature of the ultra-low temperature freezer and were subsequently semi-stoppered using siliconized Helvoet FM460 bromobutyl stoppers. The place was then placed on pre-cooled (−50° C.) shelves of a traditional batch-freeze-drying system. The freeze-drying conditions as summarized in the following table were used to transform the liquid vaccines into a solid vaccine. At the end of the conventional freeze-drying cycle, the drying chamber was backfilled with dry nitrogen until a 900 mbar chamber pressure was reached. Semi-stoppered vials were stoppered and unloaded directly after the end of the secondary drying. Vials were sealed with aluminum flip off caps and stored at −80° C. until further analysis.

| | | Shelf | Chamber | |
|---|---|---|---|---|
| | Time | temperature | pressure | Ramp rate |
| Cycle step | (hours) | (° C.) | (mbar) | (° C./min) |
| Sample loading | / | −50 | 1013 | 1 |
| Primary drying | 9 | −29 | 0.076 | 0.5 |
| Secondary drying | 5 | 25 | 0.076 | 0.1 |
| Stoppering | / | 25 | 900 | / |

The thermostability of the lyophilizates was assessed by incubation the samples for 4 weeks at 25° C. Every 7 days, samples were collected and analyzed to determine the percentage of BHK cells expressing eGFP and change in physicochemical properties of the lyophilized mRNA-LNPs.

### Study 2 Results

All lyophilizates had a uniform appearance without any minor or major defects. Reconstitution was achieved in less than 10 seconds without additional swirling. The reconstitutes were uniform and translucent just like the freshly formulated mRNA-LNP solution.

The residual moisture content post-lyophilization was consistent across all three lyophilized formats (0.3%). When incubated for 4 weeks at 25° C. the moisture content increases from 0.3% to 1% across all three lyophilized formats.

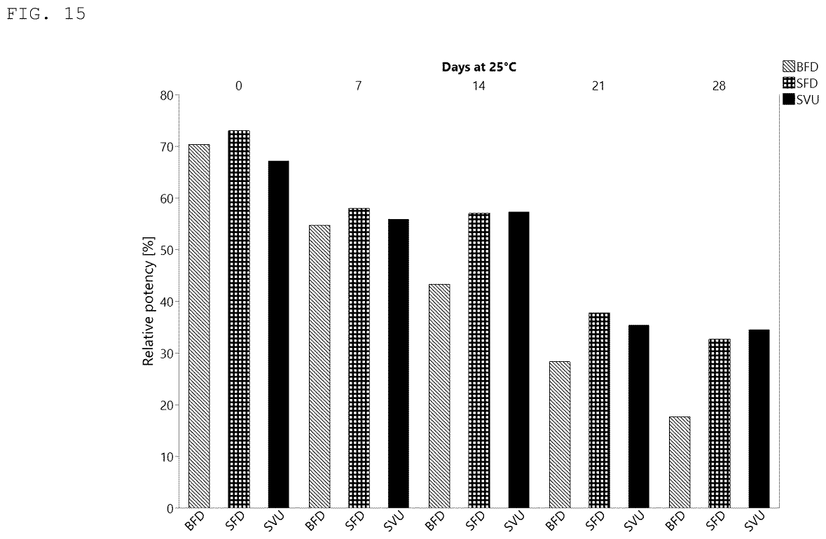

Generally, freeze-drying (i.e. across all freeze-drying conditions including spin freeze-drying, batch freeze-drying, and shelf-freeze drying) reduces the percentage of BHK cells expressing eGFP by about 30% when the measures are normalized to that of the non-lyophilized comparative stored at −80° C.—(see FIG. 15). Batch freeze-drying underperformed the other freeze-drying conditions. Those that were batch freeze-dried also underperformed the spin-lyophilized formats when incubated at 25° C. for each of the 4 weeks when evaluating the potency relative to that of the non-lyophilized conditions. The potency after 4 weeks at 25° C., for the spin-lyophilizates and batch lyophilizates are respectably 40% and 20% compared to compared to fresh mRNA-LNP samples (stored at −80° C.).

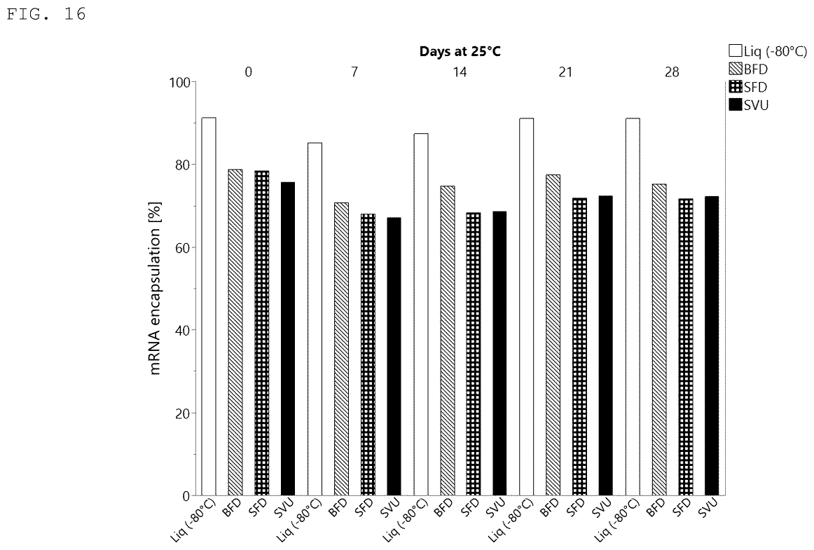

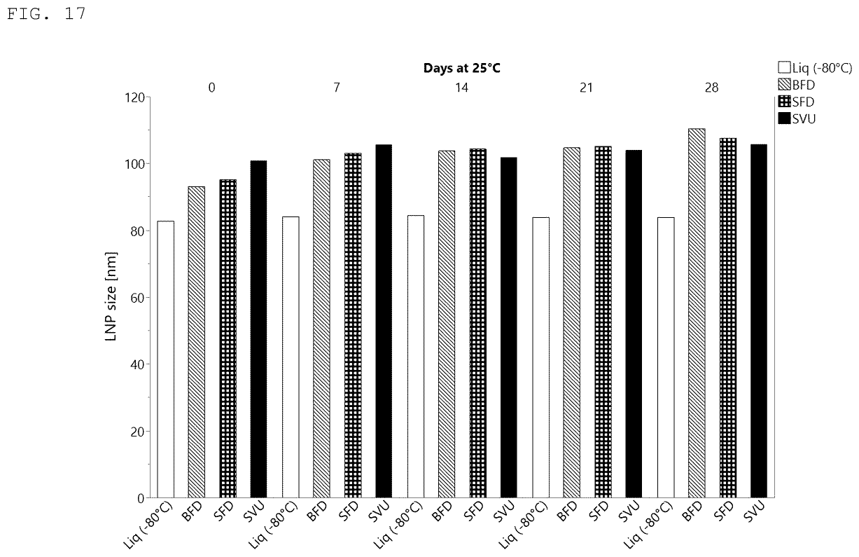

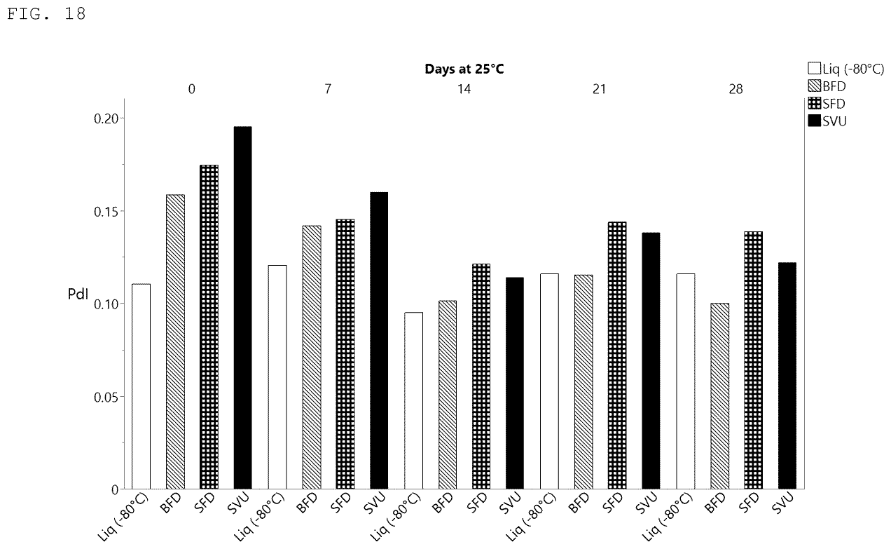

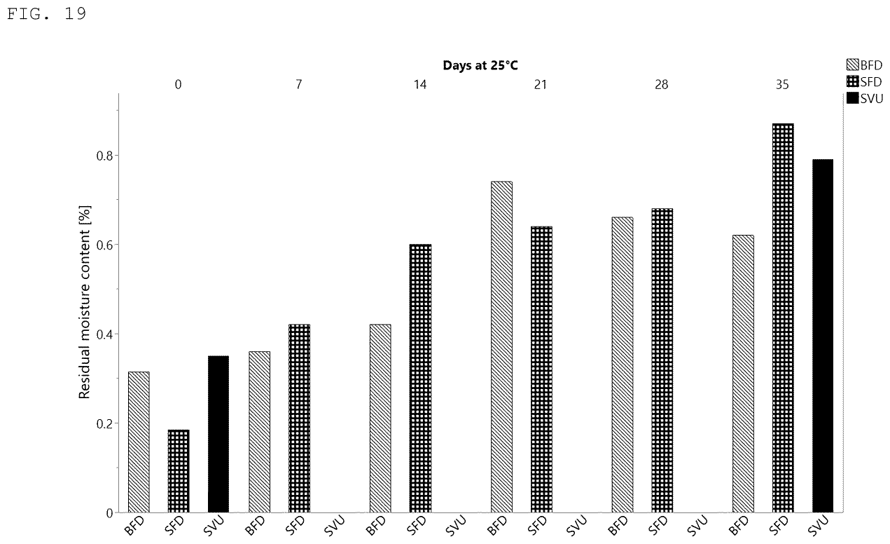

The initial drop biological activity post-lyophilization is at least partially caused by the change in physical properties of the mRNA-LNP. Initial dehydration reduces encapsulation efficiency by about 10% (see FIG. 16) while increasing the hydrodynamic diameter of the nanoparticles by about 10 nm (see FIG. 17) when compared to a freshly prepared mRNA-LNP solution. LNP size and the percentage of encapsulated mRNA remained stable over time and therefore cannot explain the significant decline the biological activity. The polydispersity index of the samples did decrease over time (FIG. 18). Integrity of the mRNA integrity and % of intact mRNA, as analyzed by capillary gel electrophoresis, declines when lyophilized mRNA-vaccines are incubated at the elevated temperature, and the quality metrics of the mRNA itself, and not its encapsulation, best corresponds with the reductions in reporter gene expression over time. The initial freeze-drying preserves the integrity of the mRNA and percentage of intact mRNA and generally preserves the LNP-mRNA vaccine under conditions superior to that of not freeze-drying (when comparing freeze-dried and non-freeze-dried samples at the same storage temperatures). Residual moisture content increased with longer storage at 25° C. (FIG. 19).

### Interpretation of Sequence Listing

Any spacing is to be ignored (sequence to be read as one continuous sequence). Uracil (“u”) residues in the below RNA sequences are denoted as “t” in the attached SEQ LISTING (ST. 26).

In the paper copy and ST.26-format electronic copy of the Sequence Listing, it is to be understood that any “U” or “u” (uridine) or “T” or “t” (thymine) depicted therein may be replaced with any of the uridine-substitutable modified nucleotides noted above, including a N1-methylpseudouridine, pseudouridine, N1-ethylpseudouridine, and that the percentage “u” replaced with uridine-substitutable modified nucleotides corresponds with those described in the embodiments above. For example, a mole percentage of the N1-methylpseudouridines to the total of the N1-methylpseudouridines and the uridines of 50% contemplates and supports substitution of 50% of the “u” with “N1T.” For example, a mole percentage of the N1-methylpseudouridines to the total of the N1-methylpseudouridines and the uridines of 25% contemplates and supports substitution of 25% of the “u” with “N1T.” For example, a mole percentage of the N1-methylpseudouridines to the total of the N1-methylpseudouridines and the uridines of 75% contemplates and supports substitution of 75% of the “u” with “N1T.” In the paper copy and ST.26-format electronic copy of the Sequence Listing, it is to be understood that any “A” or “a” (adenosine) depicted therein may be replaced with any of the adenosine-substitutable modified nucleotides noted above, and that the percentage “A” or “a” replaced with adenosine-substitutable modified nucleotides corresponds with those described in the embodiments above. In the paper copy and ASCII-format electronic copy of the Sequence Listing, it is to be understood that any “T” or “(thymidine) or “U” or “u” (uridine) depicted therein may be replaced with any of the thymidine-substitutable modified nucleotides noted above, and that the percentage “T” or “t” replaced with adenosine-substitutable modified nucleotides corresponds with those described in the embodiments above. In the paper copy and ST.26-format electronic copy of the Sequence Listing, it is to be understood that any “C” or “c” (cytosine) depicted therein may be replaced with any of the cytosine-substitutable modified nucleotides noted above, and that the percentage “C” or “c” replaced cytosine-substitutable modified nucleotides corresponds with those described in the embodiments above. In the paper copy and ASCII-format electronic copy of the Sequence Listing, it is to be understood that any “G” or “g” (guanosine) depicted therein may be replaced with any of the guanosine-substitutable modified nucleotides noted above, and that the percentage “G” or “g” replaced guanosine-substitutable modified nucleotides corresponds with those described in the embodiments above.

1. A pharmaceutical composition comprising a nucleic acid and lipid nanoparticles (LNPs); the pharmaceutical composition being lyophilized;
wherein:
(a) the percentage of nucleic acid that is encapsulated within the LNPs is greater than 75% of the total nucleic acid in the pharmaceutical composition, wherein the percentage of nucleic acid that is encapsulated within the LNPs is assessed following reconstitution of the lyophilised pharmaceutical composition;
(b) the percentage of nucleic acid that is encapsulated within the LNPs is reduced by less than 15% compared to the percentage of nucleic acid that is encapsulated within the LNPs in the pharmaceutical composition prior to lyophilisation, wherein the percentage of nucleic acid that is encapsulated within the LNPs is assessed following reconstitution of the lyophilised pharmaceutical composition and is determined by comparing the signals of a nucleic acid-binding fluorescent dye in the absence and presence of a detergent,
wherein in the absence of the detergent, the signal comes only from unencapsulated nucleic acid and
wherein in the presence of the detergent, the lipid carrier particle is disrupted so that the signal comes from the total nucleic acid (both encapsulated and non-encapsulated); or
(c) both (a) and (b).

2. The pharmaceutical composition of claim 1, wherein the lyophilised pharmaceutical composition comprises a polydispersity index (PDI) of less than 0.8 as assessed by dynamic light scattering (DLS) analysis, wherein the PDI is assessed following reconstitution of the lyophilised pharmaceutical composition.

3. (canceled)

4. The pharmaceutical composition of claim 1, wherein said pharmaceutical composition is contained within a vessel, optionally wherein the lyophilised pharmaceutical composition is coated in a layer of substantially uniform thickness against an inner surface of a circumferential wall of the vessel.

5. The pharmaceutical composition of claim 1, wherein the nucleic acid is RNA, optionally mRNA.

6. The pharmaceutical composition of claim 1, wherein RNA is present at a concentration of between 5-250 μg/ml.

7. The pharmaceutical composition of claim 1, wherein the LNPs comprise a cationic lipid, a neutral lipid, and a PEGylated lipid and, optionally wherein the LNPs further comprise cholesterol, optionally wherein the cholesterol is present from 40% to 60% (w/v).

8. The pharmaceutical composition of claim 1, wherein the pharmaceutical composition is lyophilised from an aqueous composition comprising said nucleic acid and LNPs, optionally wherein the aqueous composition further comprises sucrose in a concentration of 5% to 30% (w/v).

9. The pharmaceutical composition of claim 8, wherein the aqueous composition further comprises a salt, optionally sodium chloride, optionally sodium chloride in a concentration of between 0.1 and 50 mM.

10. (canceled)

11. A method of reconstituting the pharmaceutical composition of claim 1, the method comprising adding a sterile aqueous reconstitution solution to the pharmaceutical composition; and reconstituting the pharmaceutical composition.

12. A kit comprising the pharmaceutical composition of claim 1, the kit comprising:
(a) a first container comprising the pharmaceutical composition of claim 1; and
(b) a second container comprising a sterile aqueous reconstitution solution.

13. A method for producing the pharmaceutical composition of claim 1,
said method comprising the following steps:
i) increasing the surface area to volume ratio of an aqueous composition comprising nucleic acid and LNPs,
ii) subjecting the aqueous composition to freezing conditions sufficient to freeze the aqueous composition, and
iii) drying the frozen composition.

14. The method of claim 13, wherein, prior to step i), the aqueous composition is formulated and transferred into a vessel(s), optionally wherein between: 0.1 and 1.5 mL of said aqueous composition is transferred into the vessel(s).

15. The method of claim 13, wherein the method is semi-continuous or continuous.

16. The method of claim 13, wherein, during step i), the surface area to volume ratio is increased by greater than 1.5 times compared to the surface to area volume prior to step i).

17. The method of claim 13, wherein increasing the surface area to volume ratio of the aqueous composition is achieved by rotating a vessel containing the aqueous composition at a speed not less than that required to form and maintain the aqueous composition in a layer of substantially uniform thickness against an inner surface of a circumferential wall of the vessel, optionally wherein the vessel containing the aqueous composition is rotated about its longitudinal axis, optionally wherein the vessel containing the aqueous composition is rotated about its longitudinal axis whilst supported in a substantially horizontal orientation.

18. The method of claim 17, wherein the vessel containing the aqueous composition is rotated at a speed of between 2000 and 4000 rpm.

19. The method of claim 13, wherein, during step ii), freezing is achieved by contacting the vessel containing the aqueous composition with an inert cooling gas.

20. The method of claim 13, wherein, during step iii), the frozen composition is subjected to a primary drying step, and after the primary drying step, is subjected to secondary drying step, both primary and secondary drying steps taking place within a drying chamber, optionally wherein the primary drying step is immediately followed by the secondary drying step.

21. The method of claim 20, wherein the primary and secondary drying steps combined take less than 24 hours.

22. A freeze-dried composition obtained from the method of claim 13.

23. A kit comprising the vaccine of claim 10, the kit comprising:
(a) a first container comprising the vaccine of claim 10 and
(b) a second container comprising a sterile aqueous reconstitution solution.
